(19) United States
(12) Patent Application Publication
(10) Pub. No.: US 2023/0302134 A1
(43) Pub. Date: Sep. 28, 2023

(54) COMPOSITIONS AND METHODS FOR ENGINEERING AND SELECTION OF CAR T CELLS WITH DESIRED PHENOTYPES

(71) Applicant: Yale University, New Haven, CT (US)

(72) Inventors: Sidi Chen, Milford, CT (US); Xiaoyun Dai, West Haven, CT (US); Yaying Du, New Haven, CT (US); Jonathan Park, Scott AFB, IL (US)

(21) Appl. No.: 18/041,504

(22) PCT Filed: Aug. 13, 2021
(86) PCT No.: PCT/US2021/045882

§ 371 Date: Feb. 13, 2023

Related U.S. Application Data
(60) Provisional application No. 63/065,194, filed on Aug. 13, 2020.

(51) Int. Cl. A61K 39/00; C07K 14/725; C07K 16/28; A61P 35/00; C12N 15/10; C12N 15/86; C12N 15/11; C12N 9/22; C12N 15/90

(52) U.S. Cl. CPC A61K 39/4631; C07K 14/7051; A61K 39/4611; A61K 39/4643; C07K 16/2803; A61P 35/00; C12N 15/1082; C12N 15/1079; C12N 15/86; C12N 15/11; C12N 9/22; C12N 15/907; A61K 2039/505; C12N 2750/14143; C12N 2310/20; C12N 2800/80

(57) ABSTRACT

Compositions and methods for cellular genome engineering that permit simple and efficient targeted knock-in of a CAR and simultaneous knockout of individual genes are described. The compositions and methods are especially applicable to massively parallel engineering, selection, and identification of CAR T cell variants exhibiting a desired phenotype. AAV vectors containing crRNA and CAR expression cassettes and homology arms for targeted genomic integration thereof are provided. Also provided are libraries containing a plurality of AAV vectors and methods of use thereof in screens for identifying desirable CAR T cell variants. Methods of treatment using CAR T cell variants exhibiting improvements in one or more phenotypes are also provided.

### CROSS-REFERENCE TO RELATED APPLICATIONS

This application claims the benefit of and priority to U.S. Provisional Application No. 63/065,194 filed Aug. 13, 2020, which is hereby incorporated by reference in its entirety.

### STATEMENT REGARDING FEDERALLY SPONSORED RESEARCH

This invention was made with government support under CA238295, CA231112, and CA225498 awarded by National Institutes of Health. The government has certain rights in the invention.

### REFERENCE TO THE SEQUENCE LISTING

The Sequence Listing submitted as a text file named “YU_7927_PCT_ST25” created on Aug. 12, 2021, and having a size of 2,557,417 bytes is hereby incorporated by reference pursuant to 37 C.F.R. § 1.52(e)(5).

### FIELD OF THE INVENTION

The invention is generally related to the fields of gene editing technology and immunotherapy, and more particularly to methods of engineering improved chimeric antigen receptor T cells.

### BACKGROUND OF THE INVENTION

Cell therapies such as chimeric antigen receptor T cells (CAR-Ts) have proven to be powerful cancer therapeutics. CAR-T cell adoptive transfer therapy has demonstrated remarkable efficacy in the treatment of hematological cancers, particularly in B cell leukemia and lymphoma (Neelapu S S., et al., N Engl J Med., 377(26):2531-2544 (2017); Porter D L., et al., N Engl J Med., 365:725-733 (2011)), and has been approved by the Food and Drug Administration (FDA). An explosion of CAR-T and other forms of adoptive T cell therapies are underway. There are over 1,000 active clinical trials and numerous studies in pre-clinical stages (Tang J., et al., Nat Rev Drug Discov., 17(11):783-784 (2018)). These include CAR-Ts targeting a number of different cancer antigens; for example, CD19 and CD22-targeting CARs for B cell malignancies (Fry T J., et al., Nat Med., 24(1):20-28 (2018); Porter et al., 2011), B cell maturation antigen (BCMA)-targeting CAR for multiple myeloma, and other CARs for a number of solid tumor targets such as Mesothelin, HER2 and EGFRvIII (Ahmed N., et al., JAMA Oncology 3:1094-1101 (2017); Raje N., et al., N Engl J Med., 380:1726-1737 (2019). New CAR-T forms have recently emerged on a variety of targets such as NKG2D, MUC1, CD20, CD30, CD33, CD133, Claudins and others, although these CAR-Ts are at earlier stages of development (Brudno, J N. and Kochenderfer J N., Nature reviews Clinical oncology 15, 31 (2018); Reinhard K., et al, Science 367, 446-453 (2020)). These studies unveiled a broad landscape of CAR-T based immunotherapies across a wide range of oncology indications (June C H., et al., Science 359, 1361-1365 (2018)).

Despite the current success, there remains major challenges for CAR-T therapy. No CAR-T therapy has been approved by the FDA for solid tumors so far. Even in liquid cancers, despite high response rates, a large fraction of patients relapse owing to poor CAR-T cell expansion, persistence or loss of specific antigens (Porter D L., et al., Sci Transl Med. 7(303):303ra139 (2015); Gardner R., et al., Blood, 27(20):2406-10 (2016)). Multiple hurdles exist for CAR-T therapy, including antigen loss, metabolic suppression in the tumor microenvironment, insufficient T cell trafficking to the cancer site, lack of effective cancer cell killing, severe toxicity such as cytokine release syndrome (CRS), sub-optimal levels of T cell proliferation, and, as often observed in the clinic, failure of CAR-T persistence (June et al., 2018).

Many efforts have been invested to improve these features and to enhance CAR-T function. Examples include re-structuring of signaling domains (Sadelain M., et al., Nature 545, 423-431 (2017)), engineering of various CAR-T components such as single chain variable fragment (scFv) or transmembrane regions (Sadelain et al., 2017), overexpression of boosting factors (Lynn R C., et al., Nature 576, 293-300 (2019)), and co-administration of immunomodulating factors or viral vectors (Ma L., et al., Science 365, 162-168 (2019)), among others. Several studies have tested improvement in CAR-T cell function and persistence by changing costimulatory domains or lowering CAR binding affinity (Ghorashian S., et al., Nature Medicine 25, 1408-1414 (2019); Savoldo B., et al., The Journal of Clinical Investigation 121, 1822-1826 (2011)). Nevertheless, persistence remains a major challenge for CAR-T cells.

Thus, there is an urgent need for approaches to engineer improved CAR-Ts, allowing for CAR T therapy that shows reduced risk of immune rejection, reduced exhaustion, and enhanced persistence and effector function.

Therefore, it is an object of the invention to provide compositions and methods for engineering improved CAR T cells.

It is another object of the invention to provide CAR T cells that exhibit more stable CAR expression.

It is another object of the invention to provide CAR T cells that are more persistent in culture and in vivo.

It is yet another object of the invention to provide CAR T cells that exhibit increased cytotoxic activity and reduced exhaustion.

It is a further object of the invention to provide methods for efficient, high-throughput CAR-T engineering for the generation and selection of CAR T variants with a desired phenotype.

### SUMMARY OF THE INVENTION

Compositions and methods for cellular genomic engineering (e.g., T cell engineering) that permit simple and efficient targeted knock-in of a CAR and simultaneous knockout of individual genes are provided. The compositions and methods are especially applicable to massively parallel engineering, selection, and identification of CAR T cell variants exhibiting a desired phenotype (e.g., improved persistence) and their subsequent use in CAR-T therapy.

Provided is an AAV vector including one or more inverted terminal repeat (ITR) sequences, a 5′ homology arm, a crRNA expression cassette, a chimeric antigen receptor (CAR) expression cassette, and a 3′ homology arm. Typically, the crRNA expression cassette includes a promoter (e.g., U6) operationally linked to a sequence encoding one or more guide RNAs. The CAR expression cassette can include a promoter (e.g., an EFS promoter) and/or a polyadenylation signal sequence operationally linked to the sequence encoding the CAR. Preferably, the crRNA and CAR expression cassettes are positioned between the 5′ and 3′ homology arms. In some embodiments, the homology arms are homologous to a site in the TRAC locus.

In some embodiments, the crRNA expression cassette encodes two guide RNAs, a first guide RNA and a second guide RNA. In some embodiments, the first guide RNA targets a site in the TRAC locus while the second guide RNA targets any site in the genome. For example, the second guide RNA can target a gene involved in T cell exhaustion, T cell proliferation, T cell co-stimulation, memory T cell differentiation, T cell receptor signaling, epigenetic regulation, adaptive immune response, immune response to tumor cells, other immune functions, or combinations thereof.

The CAR can be designed to target (e.g., recognize or bind to) any desired antigen or ligand. Preferably, the CAR targets one or more cancer specific or cancer associated antigens. In some embodiments, the CAR is an anti-CD19 CAR or anti-CD22 CAR.

In some embodiments, the AAV vector includes the nucleotide sequence of SEQ ID NO:1 or SEQ ID NO:2. In some embodiments, the AAV vector includes a sequence having 75% or more sequence identity to SEQ ID NO:1 or SEQ ID NO:2. The AAV vector used in the compositions and methods can be a naturally occurring serotype of AAV or an artificial variant. In preferred embodiments, the serotype of the AAV vector is AAV6 or AAV9.

Libraries of AAV vectors are also described. The library can contain a plurality of the AAV vectors. In some embodiments, each vector in the library independently contains a crRNA expression cassette encoding a first guide RNA and a second guide RNA. In preferred embodiments, all the vectors in the library have the identical first guide RNA (e.g., a guide RNA targeting the TRAC locus). In some embodiments, each vector in the library contains a second guide RNA that is unique across the plurality of AAV vectors. The library can collectively contain from about 100 to about 300,000, from about 1,000 to about 5,000 or from about 5000, to about 10,000 distinct guide RNAs.

Cells containing the vectors and libraries thereof are also provided. For example, a population of cells can contain any of the aforementioned vectors. Populations of cells collectively containing the library are also provided. In some embodiments, each cell in the population contains at most one or two AAV vectors included in the library.

Methods of using the vectors and libraries thereof are also described. For examples, the vectors and libraries can be used to perform high-throughput screening. An exemplary method includes identifying one or more genes that enhance a desired phenotype of a cell containing a CAR. Typically, the method involves (a) contacting a population of cells collectively containing a library of vectors with an RNA-guided endonuclease under conditions suitable for genomic integration of the crRNA and CAR expression cassette and expression of the guide RNAs and CAR encoded therein; and (b) selecting for cells exhibiting the desired phenotype. In preferred embodiments the crRNA and CAR expression cassettes are integrated into the TRAC locus.

The RNA-guided endonuclease can be introduced to the cells via a viral vector that encodes the RNA-guided endonuclease, or direct electroporation of the endonuclease protein or endonuclease protein-RNA complex. The RNA-guided endonuclease can also be provided as an mRNA that encodes the RNA-guided endonuclease. The mRNA can contain modifications such as N6-methyladenosine (m6A), 5-methylcytosine (m5C), pseudouridine (ψ), N1-methylpseudouridine (me1ψ), and 5-methoxyuridine (5moU); a 5′ cap; a poly(A) tail; one or more nuclear localization signals; or combinations thereof. The mRNA can be codon optimized for expression in a eukaryotic cell and can for example, be introduced to the cells via electroporation, transfection, and/or nanoparticle mediated delivery. A preferred RNA-guided endonuclease is Cpf1, or a variant, derivative, or fragment thereof, such as, for example, Cpf1 derived from Francisella novicida U112 (FnCpf1), Acidaminococcus sp. BV3L6 (AsCpf1, including improved variants such as enAsCpf1), Lachnospiraceae bacterium ND2006 (LbCpf1), Lachnospiraceae bacterium MA2020 (Lb2Cpf1), Lachnospiraceae bacterium MC2017 (Lb3Cpf1), Moraxella bovoculi 237 (MbCpf1), or Prevotella disiens (PdCpf1).

The methods are suitable for identification of cells that exhibit any desirable features or phenotypes. Exemplary phenotypes that can be screened or selected for include increased tumor/tumor microenvironment infiltration, increased or optimized target cell affinity, increased target cell cytotoxicity, increased persistence, increased expansion/proliferation, reduced exhaustion, increased anti-cancer metabolic function, increased ability to prevent immune escape, reduced unspecific cytokine production, reduced off-target toxicity, reduced cytokine release syndrome (CRS) (e.g., when introduced in vivo), and combinations thereof. In some embodiments, cells having the desired phenotype are selected for by co-culturing the population of cells with target cells for any time period suitable for adequate selection. This can be a defined time period, such as from about 1 to about 60 days. The cells can be repeatedly co-cultured during this time period (e.g., new batches of target cells can be periodically added to the co-culture). Typically, the target cells express one or more antigens recognized by the CAR. In some embodiments, the target cells are cancer cells. In some embodiments, cells having the desired phenotype are selected for by flow cytometry-based or affinity-based sorting, immune marker-based selection, in vivo tumor infiltration (e.g., exposing the population of cells to target cells, such as tumor cells, within a subject for a time period permitting adequate selection), CAR-antigen interaction, directed evolution, or combinations thereof.

The method can additionally include identifying the crRNA expression cassette present in the cells that have been selected. Such identification can be achieved by sequencing the genomic DNA of the selected cells (e.g., at or near the region of genomic integration). Once the crRNA expression cassette present in the selected cells is known, the genes that enhance the desired phenotype can be identified as genes targeted by the guide RNAs encoded by the crRNA expression cassette.

The cells used in the compositions and methods can be T cells (e.g., CD8+ T cells such as effector T cells, memory T cells, central memory T cells, and effector memory T cells; CD4+ T cells such as Th1 cells, Th2 cells, Th3 cells, Th9 cells, Th17 cells, Tfh cells, and Treg cells; or gamma-delta T cells/gdT cells), hematopoietic stem cell (HSC), macrophages, natural killer cells (NK), B cells, dendritic cells (DC), or other immune cells.

Isolated cells that, for example, can be modified according to the foregoing methods are described. For example, an isolated CAR T cell expressing a CAR and also having one or more mutations in one or more genes identified by the screening method above is provided. In some embodiments, the cell can be selected for and isolated by the screening method or the cell can be independently generated by modifying a cell to express a CAR of interest and to contain one or more mutations in one or more genes identified by the screen. The mutation(s) can cause partial or complete loss of function of the genes or gene products thereof. In some embodiments, the CAR T cell contains one or more mutations in one or more genes selected from Table 2 or Table 3. In preferred embodiments, the CAR T cell contains one or more mutations in genes selected from PRDM1, DPF3, SLAMF1, TET2, HFE, PELI1, PDCD1, HAVCR2/TIM3, TET2, NR4A2, LAIR1, USB1 and combinations thereof. In some embodiments, the isolated CAR T cell exhibits one or more desirable phenotypes (e.g., a phenotype selected or screened for with the provided methods). For example, the cell can exhibit increased memory, increased cell proliferation, increased persistence, increased cytotoxicity towards a target cell (e.g., cancer cell), decreased T cell terminal differentiation, and/or reduced T cell exhaustion compared to a CAR T cell not including the mutations in the one or more genes. A population of cells can be derived by expanding the isolated cells. Pharmaceutical compositions containing the population of cells with a pharmaceutically acceptable buffer, carrier, diluent or excipient are also provided.

Methods of treatment are also provided. An exemplary method involves treating a subject having a disease, disorder, or condition by administering to the subject an effective amount of the aforementioned pharmaceutical composition. In some embodiments, the disease, disorder, or condition is associated with an elevated expression or specific expression of an antigen. Typically, the cells in the composition (e.g., CAR T cells) express a CAR that specifically targets the antigen. The cells for use in accordance with the compositions and methods can be derived from any appropriate source. For example, in some embodiments, the cells can be obtained from a healthy donor. In some embodiments, the cells can be obtained from the subject having the disease, disorder, or condition. Preferably the cells are obtained from the donor or subject prior to undergoing genetic modification to express the CAR and to contain the desired mutation(s) in the one or more genes.

In some embodiments, the disease, disorder, or condition is a cancer, an inflammatory disease, a neuronal disorder, HIV/AIDS, diabetes, a cardiovascular disease, an infectious disease, or an autoimmune disease. Exemplary cancers include, but are not limited to, a leukemia or lymphoma such as chronic lymphocytic leukemia (CLL), acute lymphocytic leukemia (ALL), acute myeloid leukemia (AML), chronic myelogenous leukemia (CML), mantle cell lymphoma, non-Hodgkin's lymphoma, and Hodgkin's lymphoma.

Preferably, the subject to be treated in accordance with any of the foregoing methods of treatment can be a human.

### BRIEF DESCRIPTION OF THE DRAWINGS

FIG. 1 shows establishment and characterization of the CLASH system for mass chimeric antigen receptor (CAR) engineering in human primary T cells. FIG. 1 is a schematic representation of the AAV construct design for CLASH. The crRNA expression cassette and CAR-expression cassette were inserted between the left and right TRAC homology arms in the AAV backbone. FIG. 1 also shows a schematic representation of CLASH mediated simultaneous CAR-T and Descartes library knock-in into the TRAC locus in human primary CD8 T cells. The human primary CD8 T cells were transduced with AAV-CLASH Descartes-Lib AAV6 after electroporation with Cas12a mRNA for 4 hours. The crRNAs and CAR transgenes show parallel integration into the TRAC locus by AAV-mediated HDR.

FIG. 2A is a schematic showing the Rene and Descartes library design. Diagrams of immune gene category circles are not drawn to scale. The Descartes library contains the entire Rene library as well as additional immune gene sets and additional non-targeting control (NTC) crRNAs. FIG. 2B is a schematic representation of the CAR-T cell persistence screen by continuous co-culture with NALM6 cells. CAR-T cells were co-cultured with NALM6 at E:T ratio=0.2:1 for 8 rounds after electroporation. The stimulation timepoints are indicated by the time line. FIGS. 2C-2H are bar graphs showing quantification of memory, cytotoxic and exhaustion marker expression on vector and Descartes-Lib CAR-T cells after repeated co-culture with NALM6. Quantification of the percentage of CD45RO⁺CCR7⁺ (FIG. 2C), IFNγ⁺ (FIG. 2D), TNFα⁺ (FIG. 2E), PD-1⁺ (FIG. 2F), LAG3⁺ (FIG. 2G), and TIGIT (FIG. 2H) cells are shown in each group (infection replicates, n=3). Unpaired two-sided t tests were used to assess significance. * p<0.05, *** p<0.001. Data are shown as mean±s.e.m.

FIGS. 3A-3B are graphs showing screen analysis from day 32 (FIG. 3A) or day 54 (FIG. 3B) samples vs day 0 samples using difference in log normalized crRNA abundance. FIGS. 3C-3K are graphs showing quantification of CD45RO⁺CCR7⁺ (FIG. 3C, FIG. 3D, FIG. 3E), IFNγ⁺ (FIG. 3F, FIG. 3G, FIG. 3H) and TNFα⁺ (FIG. 3I, FIG. 3J, FIG. 3K) CAR-T cell percentages compared to the vector control for each candidate gene. The marker expression levels were measured after electroporation for 5 days. One-way ANOVA with Dunnett's multiple comparisons test was used to assess statistical significance. FIG. 3L is a graph showing quantification of the CAR-T cells to cancer cell ratio at day 14 (pooled spleen and bone marrow samples; n=6 mice, 12 samples). Mann Whitney test was used to assess significance. FIG. 3M is a Venn diagram of overlapping top crRNAs between in vitro (day 32 and day 54) and in vivo (day 7, day 11 and day 14) CLASH-Descartes experiments. FDR=5% * p<0.05, ** p<0.01, *** p<0.001 and n.s P>0.05. Data are shown as mean±s.e.m.

FIGS. 4A-4T shows the characterization of PRDM1 mutant CAR T cells. FIG. 4A is a schematic representation of PRDM1 protein primary structure, with two different PRDM1 crRNAs' cutting sites indicated on PR domain and Zinc Finger domain, respectively. Predicted PRDM1-cr1 and PRDM1-cr2 cutting site are indicated by red arrows. The 3 qPCR probe target sites are indicated by blue arrows. FIG. 4B is a graph showing quantification of total indel percentage at the genomic locus targeted by PRDM1-cr1/cr2 in different healthy donors and CAR-T forms. Genomic DNA was isolated from CAR-T cells which were treated by vector or PRDM1-cr1/cr2 AAV6 for 5 days (technical replicates, n=3). Data are shown as mean±s.e.m. FIGS. 4C-4D show the Nextera-NGS sequencing results from showing unique variants observed at the genomic region targeted by the PRDM1-cr1 (FIG. 4C) and PRDM1-cr2 (FIG. 4D) in CAR-T cells. The percentage of total reads that correspond to each genotype is indicated on the right blue boxes. Red arrowheads indicate predicted cleavage sites. One representative sample's data was shown from 3 infection replicates. FIGS. 4E-4M are graphs showing quantification of the percentage of CD62L⁺ (FIG. 4E), CCR7⁺ (FIG. 4F), CD28⁺ (FIG. 4G), IL7RA⁺ (FIG. 4H), TNFα⁺ (FIG. 4I), IFNγ⁺ (FIG. 4J), Granzyme B⁺ (FIG. 4K), LAG3⁺ (FIG. 4L) and TIM3⁺ (FIG. 4M) cells in vector or PRDM1-cr1/cr2 mutant CAR-T cells (infection replicates, n=3). All experiments were analyzed by Two-way ANOVA with Tukey's multiple comparisons test to assess significance. * P<0.05, ** p<0.01 *** P<0.001 and n.s p>0.05, Data are shown as mean±s.e.m. FIGS. 4N-4O are graphs showing quantification of the frequency of CCR7⁺ (FIG. 4N) and CD62L⁺ (FIG. 4O) in different healthy donors (n=5) after transduction with vector or PRDM1-cr1 AAV6 for 5 days. Paired T-test was used to assess significance. ** P<0.01. FIGS. 4P-4Q are graphs showing proliferation of CLASH-generated PRDM1 and control CD22 CAR-T cells in response to stimulation with mitomycin-C pre-treated NALM6 cells after electroporation for 5 days in donor 2 (FIG. 4P) and donor 0286 (FIG. 4Q). CAR-T cells were transduced with vector or PRDM1-cr1 AAV6 separately (cell culture replicates, n=3). Two-way ANOVA was used to assess significance, *** p<0.001. Data are shown as mean±s.e.m. FIG. 4R is a graph showing time-course analysis of IFNγ protein expression in vector and PRDM1 mutant CAR-T cells in response to NALM6 cells stimulation in each round. Two-way ANOVA with Tukey's multiple-comparisons test was used to assess significance. Comparisons were also made between vector and PRDM1 groups at each time point. * P<0.05, ** P<0.01 and *** P<0.001, Data are shown as mean±s.e.m. FIGS. 4S-4T are graphs showing the cytotoxicity of vector and PRDM1 mutant CAR-T cells by kill assay after 7 rounds of co-culture with NALM6 with donor 2 (FIG. 4S) and donor 0286 (FIG. 4T). In vitro cytotoxic activity of CAR-T cells was measured by bioluminescence assay at different E/T ratios, using NALM6-GL cells stably transduced with GFP and luciferase genes as target cells. Two-way ANOVA was used to assess significance. *** P<0.001, Data are shown as mean±s.e.m.

FIGS. 5A-5L shows that PRDM1 mutant CAR-Ts have enhanced therapeutic efficacy in vivo. FIG. 5A is a schematic representation of the experimental design. To assess the anti-tumor ability of PRDM1 mutant CAR-T cells in vivo, mice were injected with 5×10⁵ NALM6-GL cells at day 0. Vector or PRDM1 mutant CAR-T cells, or non-transduced CD8 T cells were infused at day 3. Mice were imaged every 3-4 days. After termination, the spleen, blood and bone marrow were analyzed by flow cytometry. FIG. 5B is a graph showing quantification of whole-body bioluminescence signal over time, comparing normal CD8 T cells, vector and PRDM1 CAR22 cells. FIG. 5C is a graph showing quantification of whole-body bioluminescence signal over time, comparing normal T cells, vector and PRDM1-CAR19 cells. In FIGS. 5A and 5B, two-way ANOVA was used to assess significance. *** P<0.001, Data are shown as mean±s.e.m. FIGS. 5D-5F are graphs showing quantification of CAR-T cells to cancer cell ratio in blood (FIG. 5D), bone marrow (FIG. 5E), and spleen (FIG. 5F). Mann Whitney test was used to assess significance. FIGS. 5G-5I are graphs showing quantification of memory-like CAR-T (CD45RO⁺CD62L⁺) percentage in blood (FIG. 5G), bone marrow (FIG. 5H), and spleen (FIG. 5I). In FIGS. 5D-5I: vector CAR19 (n=8) and PRDM1 CAR19 (n=8). Mann Whitney test was used to assess significance. ** P<0.01, *** P<0.001 and n.s P>0.05, Data are shown as mean±s.e.m. FIGS. 5J-5L are survival curves showing the survival of leukemia-bearing animals treated either with vector or PDRM1 mutant CD22 CAR T cells on day 3 after tumor induction (FIG. 5J), vector or PDRM1 mutant CD22 CAR T cells on day 8 after tumor induction (FIG. 5K), and vector or PDRM1 mutant CD19 CAR T cells on day 3 after tumor induction (FIG. 5L). pXD60=Vector control anti-CD22 TRAC knockin CAR-T; pXD60-PRDM1=cr-PRDM1 CLASH anti-CD22 TRAC knockin CAR-T; pXD71=Vector control anti-CD19 TRAC knockin CAR-T; pXD71-PRDM1=cr-PRDM1 CLASH anti-CD19 TRAC knockin CAR-T. In FIGS. 5J-5L, a log-rank test was used to assess significance, p<0.0001.

FIG. 6A is a volcano plot of differentially expressed genes for PRDM1 mutant vs control CD22 CAR-T cells on day 33. FIGS. 6B-6C are enrichment plots showing enriched gene ontology pathways found by DAVID analysis on differentially upregulated genes (FIG. 6B) and differentially downregulated genes (FIG. 6C) for PRDM1 deficient vs control CAR-Ts at q-value threshold <1e-3 on day 33.

FIGS. 7A-7B are graphs showing quantification of the percentage of CD28 (FIG. 7A) and IL7RA (FIG. 7B) positive cells in CLASH-generated vector and PRDM1 mutant CD22 CAR-T cells at 1^{st}, 3^{rd} and 5^{th} rounds of co-culture with NALM6 cells (infection replicates, n=3). FIGS. 7C-7J are graphs showing quantification of mRNA expression by RT-PCR analysis for KLF2 (FIG. 7C), S1PR1 (FIG. 7D), TBX21 (FIG. 7E), FOXO1 (FIG. 7F), NFKB1 (FIG. 7G), STAT1 (FIG. 7H), STAT6 (FIG. 7I), CDCA7 (FIG. 7J), and BATF (FIG. 7K) in vector and PRDM1 mutant CAR-T cells at 1^{st}, 3^{nd} and 5^{th} rounds of co-culture with NALM6 cells (infection replicates, n=3). FIGS. 7L-7O are graphs showing quantification of the percentage of TIM3/HAVCR2 (FIG. 7L), LAG3 (FIG. 7M), 2B4/CD244 (FIG. 7N), and CD39/ENTPD1 (FIG. 7O) positive cells in vector and PRDM1 mutant CAR-T cells at 1^{st}, 3^{nd} and 5^{th} round co-culture with NALM6 cells (infection replicates, n=3). Expression of surface markers was evaluated by flow cytometry. All experimental data were analyzed by Two-way ANOVA with Sidak's multiple comparisons test to assess significance. * P<0.05, ** P<0.01, *** P<0.001 and n.s P>0.05, Data are shown as mean±s.e.m. FIG. 7P is a schematic showing the immunological phenotypes and associated genes in PRDM1 mutant CAR-T cells.

### DETAILED DESCRIPTION OF THE INVENTION

As a “living drug,” genetically engineered CAR-T cells show promise for potent and specific anti-tumor activity in the clinic (Porter, D L., et al., N. Engl. J. Med., 365(8): 725-733 (2011)). Transduction efficiency, transgene expression levels, and CAR stability or retention, are important aspects of CAR T cell therapy. However, in traditional lentiviral transduction, CAR-T cells tend to lose their transgenes and, therefore, the ability to recognize and destroy cancer cells (Ellis, J., Human Gene Therapy., 16:1241-1246 (2005)). Thus, engineering CAR-T persistence has become one of the most important tasks to allow CAR-Ts to take on their full power and thereby efficacy in vivo.

As shown in the Examples, a high-throughput approach has been developed to test for factors, which when engineered in CAR-T cells, can enhance their persistence and/or other desirable features. This approach addresses several technical barriers in CAR T engineering, including: (1) how to build CAR-T knock-ins in a massively parallel manner; (2) how to fairly compare between the different variants of CAR-T with a stable and standardized core CAR component across all the variants; (3) how to ensure quantitative assessment between different CAR variants at the same setting at high resolution; (4) how to target the high-probability set of CAR-T candidates to maximize the chance of evolving or selecting the most promising candidates for validation and downstream research and development.

These challenges are overcome by development of a platform for highly efficient massively parallel CAR-T engineering. This platform, Cas12a/Cpf1-based Large-scale AAV-perturbation with Simultaneous HDR-knockin (CLASH), permits rapid generation of custom-desired scales of CAR-T variants in a simple step, by Cpf1 mRNA electroporation with multi-functional pooled AAV transduction. The Examples demonstrate that the CLASH method generated a library scale of CAR-Ts each knocked into a desired locus in the genome, with one additional candidate immune regulator perturbed by the Cas12a/Cpf1 system. The library of CAR-T variants was assayed in a systematic way by using a long-term CAR-T cell and antigen specific cancer cell co-culture system, thereby identifying candidate CAR-T variants that have long-term persistence. Re-engineering and validation of these top variants individually showed that they enhanced CAR-T cell persistence by increasing memory-like surface markers and/or cytotoxic cytokine release. Among these, a PRDM1-mutant CAR-T increased the memory cell potential, longevity, proliferation and persistence in vivo, translating into therapeutic efficacy in a mouse model of leukemia. CLASH thus demonstrates rapid, efficient and highly scalable engineering of CAR-Ts for streamlined optimization of persistence, while maintaining versatility for application to other desired features.

### I. Definitions

“Introduce” in the context of genome modification refers to bringing into contact. For example, to introduce a gene editing composition (e.g., containing an RNA-guided endonuclease or AAV vector) to a cell is to provide contact between the cell and the composition. The term encompasses penetration of the contacted composition to the interior of the cell by any suitable means, e.g., via transfection, electroporation, transduction, gene gun, nanoparticle delivery, etc.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or are homologous, then the two sequences are 60% homologous. By way of example, the DNA sequences ATTGCC and TATGGC share 50% homology. Generally, a comparison is made when two sequences are aligned to give maximum homology.

The term “operably linked” or “operationally linked” refers to functional linkage between a regulatory sequence and a heterologous nucleic acid sequence permitting them to function in their intended manner (e.g., resulting in expression of the latter). The term encompasses positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter.

“Endogenous” refers to any material from or produced inside an organism, cell, tissue or system. “Exogenous” refers to any material introduced from or produced outside an organism, cell, tissue or system.

The term “expression” encompasses the transcription and/or translation of a particular nucleotide sequence driven by a promoter. “Expression vector” or “expression cassette” refers to a vector containing a recombinant polynucleotide having expression control sequences operatively linked to a nucleotide sequence to be expressed. An expression vector contains sufficient cis-acting elements for expression; other elements for expression can be supplied by the host cell or in an in vitro expression system. Expression vectors include all those known in the art, such as cosmids, plasmids (e.g., naked or contained in liposomes), phagemids, BACs, YACs, and viral vectors (e.g., vectors derived from lentiviruses, retroviruses, adenoviruses, and adeno-associated viruses) that incorporate the recombinant polynucleotide.

The term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

A “mutation” refers to a change in a nucleotide (e.g., DNA) sequence resulting in an alteration from a given reference sequence. The mutation can be a deletion, insertion, duplication, and/or substitution of at least one deoxyribonucleic acid base such as a purine (adenine and/or guanine) and/or a pyrimidine (thymine, uracil and/or cytosine). Mutations may or may not produce discernible changes in the observable characteristics (phenotype) of an subject.

The term “antigen” refers to a molecule capable of being bound by an antibody or T-cell receptor (e.g., a CAR). In some embodiments, an antigen is capable of provoking an immune response. This immune response can involve either antibody production, or the activation of specific immunologically-competent cells, or both. The skilled artisan will understand that any macromolecule, including proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen”. Furthermore, one skilled in the art will understand that an antigen need not be encoded solely by a full length nucleotide sequence of a gene. Moreover, a skilled artisan will understand that an antigen need not be encoded by a “gene” at all. An antigen can be synthesized or can be derived from a biological sample. Such a biological sample can include, but is not limited to a tissue sample, a tumor sample, a cell or a biological fluid. In the context of cancer “antigen” refers to an antigenic substance that is produced in a tumor cell, which can therefore trigger an immune response in the host. These cancer antigens can be useful as markers for identifying a tumor cell, which could be a potential candidate/target during treatment or therapy. There are several types of cancer or tumor antigens. There are cancer/tumor specific antigens (TSA) which are present only on tumor cells and not on healthy cells, as well as cancer/tumor associated antigens (TAA) which are present in tumor cells and also on some normal cells. In some embodiments, the TAA is expressed more abundantly in cancer cells than in in non-cancerous cells. In some embodiments, the chimeric antigen receptors are specific for tumor specific antigens. In some embodiments, the chimeric antigen receptors are specific for tumor associated antigens.

“Bi-specific chimeric antigen receptor” refers to a CAR that comprises two antigen binding domains, wherein the first domain is specific for a first ligand/antigen/target, and wherein the second domain is specific for a second ligand/antigen/target. In some embodiments, the ligand/antigen/target is a B-cell specific protein, a tumor-specific ligand, a tumor associated ligand, or combinations thereof. A bispecific CAR is specific to two different antigens. A multi-specific or multivalent CAR is specific to more than one different antigen, e.g., 2, 3, 4, 5, or more. In some embodiments, a multi-specific or multivalent CAR targets and/or binds three or more different antigens.

“Encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

The terms “target nucleic acid,” “target sequence,” and “target site” refer to a nucleic acid sequence to which an oligonucleotide such as a gRNA is designed to specifically hybridize. The target nucleic acid has a sequence that is complementary to the nucleic acid sequence of the corresponding oligonucleotide directed to the target. The target nucleic acid or target site can refer to the specific subsequence of a larger nucleic acid to which the oligonucleotide is directed or to the overall sequence (e.g., a gene or mRNA). The difference in usage will be apparent from context.

The term “locus” is the specific physical location of a DNA sequence (e.g. of a gene) on a chromosome. The term “locus” can refer to the specific physical location of an RNA guided endonuclease target sequence on a chromosome. Such a locus can comprise a target sequence that is recognized and/or cleaved by an RNA guided endonuclease. It is understood that a locus of interest can include a nucleic acid sequence that exists in the main body of genetic material (e.g., in a chromosome) of a cell and also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment in a genome in which it naturally occurs. The term also applies to nucleic acids which have been substantially purified from other components which naturally accompany the nucleic acid (e.g., RNA or DNA or proteins, which naturally accompany it in the cell). The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences.

In the context of cells, the term “isolated” refers to a cell altered or removed from its natural state. An isolated cell is thus in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

The terms “transformed,” “transduced,” and “transfected” encompass the introduction of a nucleic acid or other material into a cell by one of a number of techniques known in the art.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “percent (%) sequence identity” describes the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

The term “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird, a reptile, or an amphibian. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The term “inhibit” or “reduce” and other forms of the words such as “inhibiting” or “reducing” means to decrease, hinder or restrain a particular characteristic such as an activity, response, condition, disease, or other biological parameter. It is understood that this is typically in relation to some standard or expected value, but that it is not always necessary for the standard or relative value to be referred to. “Inhibits” or “reduce” can also mean to hinder or restrain the synthesis, expression or function of a protein relative to a standard or control. Inhibition/reduction can include, but is not limited to, the complete ablation of the activity, response, condition, or disease. For example, the term encompasses a 10% reduction in the activity, response, condition, disease, or other biological parameter as compared to the native or control level. In some embodiments, the reduction can be about 1 to 100%, or an integer therein, or any amount of reduction in between as compared to native or control levels.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

“Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

The terms “effective amount” or “therapeutically effective amount” mean a quantity sufficient to alleviate or ameliorate one or more symptoms of a disorder, disease, or condition being treated, or to otherwise provide a desired pharmacologic and/or physiologic effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable. For example, the material can be administered to a subject along with the selected compound without causing any undesirable biological effects or interacting in a deleterious manner with any of the other components of the pharmaceutical composition in which it is contained.

Recitation of ranges of values are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other embodiments the values can range in value either above or below the stated value in a range of approx. +/−5%.

### II. Compositions

Compositions for use in the methods are provided. For example, gene editing compositions for use in methods of modifying the genome of a cell are provided. Exemplary compositions include nucleic acid vectors, libraries thereof, and cells containing the vectors and libraries thereof. Pharmaceutical compositions containing modified cells are also provided.

A. Gene Editing Compositions

Exemplary gene editing compositions for modifying the genome of a cell include an RNA-guided endonuclease and a vector (e.g., AAV vector). The vector can contain a sequence (e.g., a crRNA expression cassette) that encodes one or more crRNAs that direct the endonuclease to one or more target genes, a sequence that encodes one or more chimeric antigen receptors (e.g., a CAR expression cassette), and/or one or more sequences homologous to one or more target sites (e.g., TRAC).

The RNA-guided endonuclease and vector can be in the same or different compositions and can be introduced to the cell together or separately. For example, an RNA-guided endonuclease and vector can be provided in different compositions that are introduced to the cell together or separately. In some embodiments, when the gene editing compositions are administered as an isolated nucleic acid or are contained within an expression vector, the RNA-guided endonuclease (e.g., Cpf1) can be encoded by the same nucleic acid or vector as the crRNA and CAR expression cassettes. Alternatively, or in addition, the RNA-guided endonuclease can be encoded in a physically separate nucleic acid or vector from the vector encoding the crRNA and CAR expression cassette.

In some embodiments, after introduction of the RNA-guided endonuclease, AAV vector can be introduced into the cells either immediately, or after a certain period of time such as, about 1 h, about 2 h, about 3 h, about 4 h, about 5 h, about 6 h, about 7 h, about 8 h, about 9 h, about 10 h, about 12 h, about 24 h, about 48 h, about 72 h, or about 96 h.

The RNA-guided endonuclease can alter (e.g., increase or reduce expression and/or activity of) one or more target genes or gene products thereof. For example, the RNA-guided endonuclease can cause disruption of one or more target genes. This disruption includes but is not limited to alterations in the genome (such as, but not limited to, insertions, deletions, duplications, translocations, DNA or histone methylation, acetylation, and combinations thereof) that can result in reduced or abolished expression and/or activity of the target gene or gene product. Methods of determining the expression and/or activity of a gene product are known in the art. These include, but are not limited to, PCR, northern blot, southern blot, western blot, nuclease surveyor assays, sequencing, ELISA, FACS, mRNA-sequencing, single-cell RNA-sequencing, and other molecular biology, chemical, biochemical, cell biology, and immunology assays. A skilled person, based on methods known in the art, and the described teachings would understand how to determine and/or confirm alteration of a target gene.

The RNA-guided endonuclease can be introduced to the cell through a variety of techniques, including viral and non-viral approaches. For example, the RNA-guided endonuclease can be introduced via a viral vector (e.g., a retrovirus such as a lentivirus, adenovirus, poxvirus, Epstein-Barr virus, or adeno-associate virus (AAV)) that encodes the RNA-guided endonuclease. Non-viral approaches such as physical and/or chemical methods can also be used, including, but not limited to cationic liposomes and polymers, DNA nanoclew, gene gun, microinjection, transfection, electroporation, nucleofection, particle bombardment, ultrasound utilization, magnetofection, conjugation to cell penetrating peptides, and/or nanoparticle mediated delivery. Such methods are described for example, in Nayerossadat N., et al., Adv. Biomed. Res., 1:27 (2012) and Lino C A, et al., Drug Deliv., 25(1):1234-1257 (2018). In view of the respective advantages and disadvantages of each method, a skilled artisan would be able to determine an optimal method for introduction of the RNA-guided endonuclease.

In preferred embodiments, the mRNA is introduced to the cell via electroporation. Electroporation is temporary destabilization of the cell membrane by insertion of a pair of electrodes into it so that nucleic acid molecules (e.g., DNA, RNA) in the surrounding media of the destabilized membrane would be able to penetrate into cytoplasm and nucleoplasm of the cell. The RNA-guided endonuclease can also be introduced via direct electroporation of the endonuclease protein or endonuclease protein-RNA complex (e.g., endonuclease protein complexed with a crRNA).

In preferred embodiments, the RNA-guided endonuclease can be provided to the cell in the form of an mRNA that encodes the RNA-guided endonuclease. The mRNA can be modified or unmodified. The mRNA can be modified for example, to reduce immunogenicity, to optimize translation, and/or to confer increased stability and/or expression of the RNA-guided endonuclease. The modified mRNA can incorporate a number of chemical changes to the nucleotides, including changes to the nucleobase, the ribose sugar, and/or the phosphodiester linkage. These modified mRNA can improve efficiency of the RNA-guided endonuclease, reduce off-target effects, reduce toxicity, increase endonuclease protein levels, increase endonuclease activity, and/or increase mRNA stability relative to the unmodified mRNA. Li, B., et al., Nat. Biomed. Eng., 1(5): pii: 0066 (2017) and WO 2017/181107 disclose compositions and methods of modifying mRNAs that can be used in accordance with the compositions and methods.

Exemplary mRNA modifications include, without limitation, N6-methyladenosine (m6A), 5-methylcytosine (m5C), pseudouridine (ψ), N1-methylpseudouridine (me1ψ), and 5-methoxyuridine (5moU), a 5′ cap, a poly(A) tail, one or more nuclear localization signals, or combinations thereof.

The mRNA can be codon optimized for expression in a eukaryotic cell (e.g., a cell derived from a plant, human, mouse, rat, rabbit, dog, or non-human mammal or primate). Codon-optimization describes gene engineering approaches that use changes of rare codons to synonymous codons that are more frequently used in the cell type of interest with the aim of increasing protein production. In general, codon optimization involves modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Thus, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al., Nucl. Acids Res., 28:292 (2000). Computer algorithms for codon optimizing a particular sequence for expression in a particular host cell are also available, such as Gene Forge (Aptagen; Jacobus, PA), are also available. In some embodiments, one or more codons in a sequence encoding an RNA-guided endonuclease corresponds to the most frequently used codon for a particular amino acid.

The gene editing compositions also include libraries, e.g., libraries of the AAV vectors. The library can be a collection of multiple vectors, which may be the same or different. In preferred embodiments, the library contains a plurality of different AAV vectors. For example, in some embodiments, all the vectors in the library have the identical first guide RNA (e.g., a guide RNA targeting the TRAC locus) while each vector in the library also contains a second guide RNA that is unique across the plurality of AAV vectors. A unique guide RNA is the only RNA of its kind in the vector or library of vectors (e.g., the guide RNA can be the only one having a particular nucleotide sequence). Collectively, the library can contain any number of guide RNAs. For example, the library can contain guide RNAs that collectively target the entire set of protein coding genes in the genome (e.g., a human genome-wide library). Alternatively, the library can contain guide RNAs that target a selected subset of genes or sites. In some embodiments, the library collectively contains a plurality of guide RNAs encoded by nucleic acid sequences selected from SEQ ID NOs:3-12,134. In preferred embodiments, the library collectively contains guide RNAs encoded by the nucleic acid sequences of SEQ ID NOs:3-4,087 (Rene library) or SEQ ID NOs:4,088-12,134 (Descartes library).

The library can contain multiple (e.g., 2, 3, 4, 5, 6, 7, 8, 9, 10, or more) unique guide RNAs targeting the same gene. In preferred embodiments, the library also contains a representative number (e.g., 1000) of non-targeting control guide RNAs. Preferably, the library contains a total number of guide RNAs that is representative of all the genes or sites of interest to be targeted. For example, the upper limit on the number of guide RNAs can be reflective of current pooled oligonucleotide synthesis and/or cloning limits (e.g., about 300,000 distinct guide RNA sequences). In some embodiments, the library contains about 100 or more distinct guide RNA sequences. In some embodiments, the library contains about 1000, 5000, 8000, 10,000, 15,000, 20,000, 30,000, 40,000, 50000, 100000, 150000, 200000, 250000, 3000000, or more distinct guide RNA sequences. In some embodiments, the library contains from about 100 to about 300000 distinct guide RNA sequences. In some embodiments, the library can be in the form of a collection of plasmids or a collection of viruses collectively containing the vectors of the library.

1. RNA-Guided Endonuclease

An “RNA-guided endonuclease” is a polypeptide whose endonuclease activity and specificity depend on its association with an RNA molecule. The full sequence of this RNA molecule or more generally a fragment of this RNA molecule has the ability to specify a target sequence in the genome. In general, this RNA molecule has the ability to hybridize a target sequence and to mediate the endonuclease activity of the RNA-guided endonuclease. Non-limiting examples of RNA-guided endonucleases include Cas1, Cas1B, Cas2, Cas3, Cas4, Cas5, Cas6, Cas7, Cas8, Cas9 (also known as Csn1 and Csx12), Cpf1, homologues thereof, or modified versions thereof. A preferred RNA-guided endonuclease is Cas12a (Cpf1), a component of the CRISPR/Cas system.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By providing a cell with the required elements including a cas gene and specifically designed CRISPRs, the genome can be cut and modified at any desired location. Methods of preparing compositions for use in genome editing using CRISPR/Cas systems are described in detail in WO 2013/176772 and WO 2014/018423, which are specifically incorporated by reference herein in their entireties.

The term “Cas” (CRISPR-associated) generally refers to an effector protein of a CRISPR-Cas system or complex. The term “Cas” can be used interchangeably with the terms “CRISPR” protein, “CRISPR-Cas protein,” “CRISPR effector,” CRISPR-Cas effector,” “CRISPR enzyme,” “CRISPR-Cas enzyme” and the like, unless otherwise apparent. The RNA-guided endonuclease can be a Cas effector, Cas protein, or Cas enzyme. In general, a “CRISPR system,” refers collectively to transcripts and other elements involved in the expression of or directing the activity of CRISPR-associated (“Cas”) genes, including sequences encoding a Cas gene, and where applicable, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is used (e.g., RNA(s) to guide Cas, such as Cas9 or Cpf1, e.g. CRISPR RNA (crRNA) and/or transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence. See, e.g., Shmakov et al. (2015) “Discovery and Functional Characterization of Diverse Class 2 CRISPR-Cas Systems,” Molecular Cell, DOI: dx.doi.org/10.1016/j.molcel.2015.10.008.

The RNA-guided endonuclease can be a Cas effector protein selected from, without limitation, a type II, type V, or type VI Cas effector protein.

In some embodiments, one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold) if needed, as well as a suitable promoter and necessary elements for proper processing in eukaryotic cells. Such vectors are commercially available (see, for example, Addgene). Many of the systems rely on custom, complementary oligomers that are annealed to form a double stranded DNA and then cloned into the sgRNA expression plasmid. Co-expression of the sgRNA and the appropriate Cas enzyme in the cell results in a single or double strand break (depending of the activity of the Cas enzyme) at the desired target site.

### Cas12a (Cpf1)

In preferred embodiments, the RNA-guided endonuclease is Cpf1. The RNA guided endonuclease can be a Cpf1 ortholog, variant, or engineered derivative, derived from any bacterial species known to contain Cpf1. For example, Cpf1 effector proteins can be derived from an organism from a genus including Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus. More particularly, in some embodiments, the RNA-guided endonuclease is a Cpf1 from one of the following organisms: S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L monocytogenes, L ivanovii; C. botulinum, C. difficile, C. tetani, C. sordellii.

In some embodiments, the Cpf1 is derived or isolated from a bacterial species selected from Francisella tularensis 1 (e.g., Francisella tularensis subsp. Novicida), Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. A preferred RNA-guided endonuclease is a Cpf1, or a variant, derivative, or fragment thereof derived from Francisella novicida U112 (FnCpf1), Acidaminococcus sp. BV3L6 (AsCpf1), Lachnospiraceae bacterium ND2006 (LbCpf1), Lachnospiraceae bacterium MA2020 (Lb2Cpf1), Lachnospiraceae bacterium MC2017 (Lb3Cpf1), Moraxella bovoculi 237 (MbCpf1), Butyrivibrio proteoclasticus (BpCpf1), Parcubacteria bacterium GWC2011_GWC2_44_17 (PbCpf1); Peregrinibacteria bacterium GW2011_GWA_33_10 (PeCpf1), Leptospira inadai (LiCpf1), Smithella sp. SC_K08D17 (SsCpf1), Porphyromonas crevioricanis (PcCpf1), Porphyromonas macacae (PmCpf1), Candidatus Methanoplasma termitum (CMtCpf1), Eubacterium eligens (EeCpf1), Moraxella bovoculi 237 (MbCpf1), or Prevotella disiens (PdCpf1). In preferred embodiments, the Cpf1 is LbCpf1, or a variant, derivative, or fragment thereof.

Cpf1 effector proteins can be modified, e.g., an engineered or non-naturally-occurring Cpf1. The modification can contain mutations of one or more amino acid residues of the effector protein. The mutations can be in one or more catalytically active domains of the effector protein (e.g., RuvC domain or a catalytically active domain which is homologous to a RuvC domain). The effector protein can have reduced or abolished nuclease activity compared with an effector protein lacking the one or more mutations. In some embodiments, the effector protein does not direct cleavage of a DNA or RNA strand at the target locus of interest.

In some embodiments, the one or more modified or mutated amino acid residues are D917A, E1006A or D1255A with reference to the amino acid position numbering of the FnCpf1 effector protein. In some embodiments, the one or more mutated amino acid residues are D908A, E993A, and D1263A with reference to the amino acid positions in AsCpf1 or LbD832A, E925A, D947A, and D1180A with reference to the amino acid positions in LbCpf1.

Mutations can also be made at neighboring residues, e.g., at amino acids near those indicated above that participate in the nuclease activity. In some embodiments, only the RuvC domain is inactivated, and in other embodiment, another putative nuclease domain is inactivated. In some embodiments, two FnCpf1, AsCpf1 or LbCpf1 variants (each a different nickase) are used to increase specificity. For example, two nickase variants can be used to cleave DNA at a target (where both nickases cleave a DNA strand, while minimizing or eliminating off-target modifications where only one DNA strand is cleaved and subsequently repaired). In some embodiments, the Cpf1 effector protein cleaves sequences associated with or at a target locus of interest as a homodimer comprising two Cpf1 RNA-guided endonucleases. In some embodiments, the homodimer can contain two Cpf1 effector proteins having different mutations in their respective RuvC domains.

In some embodiments, the Cpf1 is a wildtype protein, a humanized Cpf1, a variant, a derivative, a fragment, a shuffled domain version, or combinations thereof. In some embodiments, the RNA-guided endonuclease can be a chimeric Cpf1 effector protein having a first fragment from a first Cpf1 effector protein ortholog and a second fragment from a second Cpf1 effector protein ortholog, and wherein the first and second effector protein orthologs are different (e.g., derived from different organisms).

The Cpf1 effector protein can have one or more heterologous functional domains such as a nuclear localization signal (NLS) domain. The NLS domain(s) can be positioned at or near or in proximity to a terminus of the Cpf1 effector protein. Heterologous functional domains also include transcriptional activation domains (e.g., VP64, VPR, p65, HSF1, Activ), transcriptional repression domains (e.g., KRAB; methyl transferase domains of DNMT family members including DNMT1, DNMT3A, DNMT3B, and DNMT3L; or a SID domain (e.g. SID4X)), and nuclease domains (e.g., Fok1). The heterologous functional domains can have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity. The heterologous functional domains can be fused, linked, tethered, or otherwise associated with the RNA-guided endonuclease.

A protospacer adjacent motif (PAM) or PAM-like motif directs binding of the RNA-guided endonuclease complex to the target locus of interest. In some embodiments, the PAM is 5′ TTN, where N is A/C/G or T and the effector protein is FnCpf1p; the PAM is 5′ TTTV, where V is A/C or G and the effector protein is AsCpf1, LbCpf1 or PaCpf1p. In some embodiments, the PAM is located upstream of the 5′ end of the protospacer. The T-rich PAMs of the Cpf1 family allow for targeting and editing of AT-rich genomes.

The Cas12a effector protein can further include dCpf1 fused to an adenosine or cytidine deaminase such as those described in U.S. Provisional Application Nos. 62/508,293, 62/561,663, and 62/568,133, 62/609,949, and 62/610,065. Additional Cas12a effector proteins that can be used are discussed in International Patent Application Nos. WO 2016/205711, WO 2017/106657, and WO 2017/172682.

Given the potential toxicity of the RNA-guided endonuclease within the cells, due to possible non-specific interactions with various RNAs in the cell or off-site targeting, some approaches can be taken to induce the nuclease activity of the RNA-guided endonuclease, such as Cpf1, transiently (e.g., mRNA electroporation), ideally during the life-span of the guide RNA into the cells. In some embodiments, the RNA-guided endonuclease (such as Cpf1) can be expressed under a stabilized or inactive form, which is made active upon activation by an enzyme produced by the cell or destabilization of its polypeptide structure inside the cell. Conditional protein stability can be obtained for instance by fusion of the endonuclease to a stabilizing/destabilizing protein based, as a non-limiting example, on the FKBP/rapamycin system, where protein conformational change is induced by a small molecule. Chemical or light induced dimerization of a protein partner fused to the endonuclease protein can also be used to lock or unlock the endonuclease.

2. Vector

Suitable vectors for inclusion in the gene editing compositions or for providing elements of the gene editing compositions include, without limitation, plasmids and viral vectors derived from, for example, bacteriophages, baculoviruses, retroviruses (such as lentiviruses), adenoviruses, poxviruses, Epstein-Barr viruses, and adeno-associated viruses (AAV). The viral vector can be derived from a DNA virus (e.g., dsDNA or ssDNA virus) or an RNA virus (e.g., an ssRNA virus). Numerous vectors and expression systems are commercially available from commercial vendors including Addgene, Novagen (Madison, WI), Clontech (Palo Alto, CA), Stratagene (La Jolla, CA), and Invitrogen/Life Technologies (Carlsbad, CA).

In preferred embodiments, AAV vectors are provided as components of the gene editing compositions for modifying the genome of one or more cells. The AAV vector can provide one or more elements of the gene editing compositions (e.g., crRNA expression cassette, CAR expression cassette, homology arms).

AAV is a non-pathogenic, single-stranded DNA virus that has been actively employed over the years for delivering therapeutic genes in both in vitro and in vivo systems (Choi, et al., Curr. Gene Ther., 5:299-310, (2005)). AAV belongs to the parvovirus family and is dependent on co-infection with other viruses, mainly adenoviruses, in order to replicate. Initially distinguished serologically, molecular cloning of AAV genes has identified hundreds of unique AAV strains in numerous species. Each end of the single-stranded DNA genome contains an inverted terminal repeat (ITR), which is the only cis-acting element required for genome replication and packaging. The single-stranded AAV genome contains three genes, Rep (Replication), Cap (Capsid), and aap (Assembly). These three genes give rise to at least nine gene products through the use of three promoters, alternative translation start sites, and differential splicing. These coding sequences are flanked by the ITRs. The Rep gene encodes four proteins (Rep78, Rep68, Rep52, and Rep40), while Cap expression gives rise to the viral capsid proteins (VP; VP1/VP2/VP3), which form the outer capsid shell that protects the viral genome, as well as being actively involved in cell binding and internalization. It is estimated that the viral coat contains 60 proteins arranged into an icosahedral structure with the capsid proteins in a molar ratio of 1:1:10 (VP1:VP2:VP3).

Recombinant AAV (rAAV), which lacks viral DNA, is essentially a protein-based nanoparticle engineered to traverse the cell membrane, where it can ultimately traffic and deliver its DNA cargo into the nucleus of a cell. In the absence of Rep proteins, ITR-flanked transgenes encoded within rAAV can form circular concatemers that persist as episomes in the nucleus of transduced cells. Because recombinant episomal DNA does not integrate into host genomes, it will eventually be diluted over time as the cell undergoes repeated rounds of replication. This will eventually result in the loss of the transgene and transgene expression, with the rate of transgene loss dependent on the turnover rate of the transduced cell. These characteristics make rAAV ideal for certain gene therapy applications.

AAV can be advantageous over other viral vectors due to low toxicity (e.g., this can be due to the purification method not requiring ultra centrifugation of cell particles that can activate the immune response) and low probability of causing insertional mutagenesis because AAV does not integrate into the host genome (primarily remaining episomal).

The sequences placed between the ITRs will typically include a mammalian promoter, gene of interest, and a terminator. In many cases, strong, constitutively active promoters are desired for high-level expression of the gene of interest. Commonly used promoters of this type include the CMV (cytomegalovirus) promoter/enhancer, elongation factor 1α short (EFS), SV40 (simian virus 40), chicken β-actin and CAG (CMV, chicken β-actin, rabbit β-globin). All of these promoters provide constitutively active, high-level gene expression in most cell types. Some of these promoters are subject to silencing in certain cell types, therefore this consideration should be evaluated for each application.

In some cases it can be advantageous for a transgene (e.g., being targeted for integration) to be kept under the control of an endogenous promoter (e.g., a promoter at or near the site of integration). For example, the CAR expression cassette provided by the AAV vector can contain a splice acceptor/donor, 2A peptide, and/or internal ribosome entry site (IRES) operationally linked to a transgene (e.g., CAR) to allow expression of the transgene in frame with a gene at the site of integration and/or under the control of the promoter at the site of integration. In other cases, it can be advantageous for the transgene to be under the control of an exogenous promoter, such as a constitutive promoter or an inducible promoter. In such cases, the CAR expression cassette provided by the AAV vector can contain a promoter (e.g., EFS or tetracycline-inducible promoter) operationally linked to a transgene (e.g., reporter gene, CAR).

In some embodiments, the crRNA expression cassette and CAR expression cassette are present on one nucleic acid molecule, e.g., one AAV vector. In some embodiments, the crRNA expression cassette is present on a first nucleic acid molecule, e.g., a first AAV vector, and the CAR expression cassette is present on a second nucleic acid molecule, e.g., a second AAV vector. The first and second nucleic acid molecules can be AAV vectors, e.g., AAV6 or AAV9. In some embodiments, the RNA-guided endonuclease, crRNA expression cassette and CAR expression cassette are present on one nucleic acid molecule, e.g., an AAV vector such as AAV6 or AAV9.

The packaging limit of the vector to be used would determine the number and combinations of gene editing elements (e.g., RNA-guided endonuclease, crRNA expression cassette(s), CAR expression cassette(s), or combinations thereof) that can be provided by said vector. For example, AAV has a packaging limit of approximately 4.5 to 4.8 Kb. As such, attempts to package larger constructs can lead to significantly reduced virus production. In preferred embodiments, the RNA-guided endonuclease is introduced to the cell by a different means from the vector encoding the crRNA expression cassette(s) and/or CAR expression cassette(s). Introduction of gene editing compositions (e.g., RNA-guided endonuclease and the AAV vector(s) containing the crRNA expression cassette(s), CAR expression cassette(s)) to the cell can be performed ex vivo and at the same or different times.

In preferred embodiments, the vector is an AAV vector including (i) a crRNA expression cassette encoding one or more guide RNAs (e.g., selected from SEQ ID NOs:3-12,134); (ii) a chimeric antigen receptor (CAR) expression cassette; and (iii) 5′ and 3′ homology-directed repair (HDR) arms for targeted genomic integration. Preferably, the crRNA expression cassette encodes two guide RNAs. In some embodiments, a first guide RNA is constitutively present (e.g., a guide RNA targeting the TRAC locus). In some embodiments, the crRNA expression cassette contains one or more restriction sites (e.g., BbsI) downstream of the first guide RNA that permit insertion of any sequence of interest (e.g., a sequence encoding a second guide RNA). The sequence to be inserted can be variable, for example, the sequence can be varied depending on the gene or locus to be targeted. The presence of one or more restriction sites (e.g., BbsI) allows for the vector to be linearized, followed by ligation of a sequence encoding the guide RNA. In some embodiments, the crRNA expression cassette and CAR expression cassette are positioned between the 5′ and 3′ HDR arms, such that both cassettes undergo genomic integration at a specific target site.

An exemplary sequence of suitable AAV vector containing an anti-CD22 CAR is provided below:

cctgcaggca gctgcgcgct cgctcgctca ctgaggccgc
ccgggcaaag cccgggcgtc gggcgacctt tggtcgcccg
gcctcagtga gcgagcgagc gcgcagagag ggagtggcca
actccatcac taggggttcc tgcggccgca cgcgtgatgt
aaggagctgc tgtgacttgc tcaaggcctt atatcgagta
aacggtagtg ctggggctta gacgcaggtg ttctgattta
tagttcaaaa cctctatcaa tgagagagca atctcctggt
aatgtgatag atttcccaac ttaatgccaa cataccataa
acctcccatt ctgctaatgc ccagcctaag ttggggagac
cactccagat ttcaagatgt acagtttgct ttgctgggcc
tttttcccat gcctgccttt actctgccag agttatattg
ctggggtttt gaagaagatc ctattaaata aaagaataag
cagtattatt aagtagccct gcatttcagg tttccttgag
tggcaggcca ggcctggccg tgaacgttca ctgaaatcat
ggcctcttgg ccaagattga tagcttgtgc ctgtccctga
gtcccagtcc gtcacgagca gotggtttct aatatgctat
ttcccgtata aagcatgaga ccgtgacttg ccagccccac
agagccccgc ccttgtccat cactggcatc tggactccag
cctgggttgg ggcaaagagg gaaatgagat catgtcctaa
ccctgatcct cttgtcccac agatatccag aaccctgacc
cgcgttagcg ctagaagagg gcctatttcc catgattcct
tcatatttgc atatacgata caaggctgtt agagagataa
ttggaattaa tttgactgta aacacaaaga tattagtaca
aaatacgtga cgtagaaagt aataatttct tgggtagttt
gcagttttaa aattatgttt taaaatggac tatcatatgc
ttaccgtaac ttgaaagtat ttcgatttct tggctttata
tatcttgtgg aaaggacgaa acaccgtaat ttctactaag
tgtagatgag tctctcagct ggtacactaa tttctactaa
gtgtagatgg tcttcgagaa gacctttttt aagcttggcg
tggatccgat atcaactaga tottgagaca aggtacctag
gtcttgaaag gagtgggaat tggctccggt gcccgtcagt
gggcagagcg cacatcgccc acagtccccg agaagttggg
gggaggggtc ggcaattgat ccggtgccta gagaaggtgg
cgcggggtaa actgggaaag tgatgtcgtg tactggctcc
gcctttttcc cgagggtggg ggagaaccgt atataagtgc
agtagtcgcc gtgaacgttc tttttcgcaa cgggtttgcc
gccagaacac aggaccggtt ctagacgtac ggccaccatg
cttctgctcg tgacaagcct gctgctgtgc gagctgcccc
accctgcctt tctgctgatc cctcaggtgc agctgcagca
gtctggccct ggcctcgtga agcctagcca gaccctgagc
ctgacctgtg ccatcagcgg cgatagcgtg tccagcaata
gcgccgcctg gaactggatc agacagagcc ctagcagagg
cctggaatgg ctgggccgga cctactaccg gtccaagtgg
tacaacgact acgccgtgtc cgtgaagtcc cggatcacca
tcaaccccga caccagcaag aaccagttct ccctgcagct
gaacagcgtg acccccgagg ataccgccgt gtactactgc
gccagagaag tgaccggcga cctggaagat gccttcgaca
tctggggcca gggcacaatg gtcaccgtgt ctagcggagg
cggcggaagc gacatccaga tgacacagag ccccagctcc
ctgagcgcca gcgtgggaga cagagtgacc atcacctgtc
gggccagcca gaccatctgg tcctacctga actggtatca
gcagcggcct ggcaaggccc ccaacctgct gatctatgcc
gccagctcac tgcagagcgg cgtgcccagc agattttccg
gcagaggcag cggcaccgac ttcaccctga caatcagttc
cctgcaggcc gaggacttcg ccacctacta ctgccagcag
agctacagca tcccccagac cttcggccag gggaccaagc
tggaaatcaa agcggccgca ggtaccacca cgacgccagc
gccgcgacca ccaacaccgg cgcccaccat cgcgtcgcag
cccctgtccc tgcgcccaga ggcatgccgg ccagcagcag
ggggcgcagt gcacacgagg gggctggact tcgcctgtga
tatctacatc tgggcgccct tggccgggac ttgtggggtc
cttctcctgt cactggttat caccctttac tgcaaacggg
gcagaaagaa actcctgtat atattcaaac aaccatttat
gagaccagta caaactactc aagaggaaga tggctgtagc
tgccgatttc cagaagaaga agaaggagga tgtgaactga
gagtgaagtt cagcaggagc gcagacgccc ccgcgtacca
gcagggccag aaccagctct ataacgagct caatctagga
cgaagagagg agtacgatgt tttggacaag agacgtggcc
gggaccctga gatgggggga aagccgagaa ggaagaaccc
tcaggaaggc ctgtacaatg aactgcagaa agataagatg
gcggaggcct acagtgagat toggatgaaa ggcgagcgcc
ggaggggcaa ggggcacgat ggcctttacc agggtctcag
tacagccacc aaggacacct acgacgccct tcacatgcag
gccctgcccc ctcgctaagc tagcaataaa agatctttat
tttcattaga tctgtgtgtt ggttttttgt gtggtaccga
gagactctaa atccagtgac aagtctgtct gcctattcac
cgattttgat tctcaaacaa atgtgtcaca aagtaaggat
totgatgtgt atatcacaga caaaactgtg ctagacatga
ggtctatgga cttcaagagc aacagtgctg tggcctggag
caacaaatct gactttgcat gtgcaaacgc cttcaacaac
agcattattc cagaaaacac cttcttcccc agcccaggta
agggcagctt tggtgccttc gcaggctgtt tccttgcttc
aggaatggcc aggttctgcc cagagctctg gtcaatgatg
tctaaaactc ctctgattgg tggtctcggc cttatccatt
gccaccaaaa ccctcttttt actaagaaac agtgagcctt
gttctggcag tccagagaat gacacggaaa aaaagcagat
gaagagaagg tggcaggaga gggcacgtgg cccagcctca
gtctctccaa ctgagttcct gcctgcctgc ctttgctcag
actgtttgcc ccttactgct cttctaggcc tcattctaag
ccccttctcc aagttgcctc tccttatttc tccctgtctg
ccaaaaaatc tttcccagct cactaagtca gtctcacgca
gtcactcatt aacccacaat tcgatatcaa gcttaataaa
agatctttat tttcattaga tctgtgtgtt ggttttttgt
gtggtaacca cgtgcggacc gagcggccgc aggaacccct
agtgatggag ttggccactc cctctctgcg cgctcgctcg
ctcactgagg ccgggcgacc aaaggtcgcc cgacgcccgg
gctttgcccg ggcggcctca gtgagcgagc gagcgcgcag
ctgcctgcag gggcgcctga tgcggtattt tctccttacg
catctgtgcg gtatttcaca ccgcatacgt caaagcaacc
atagtacgcg ccctgtagcg gcgcattaag cgcggcgggt
gtggtggtta cgcgcagcgt gaccgctaca cttgccagcg
ccctagcgcc cgctcctttc gctttcttcc cttcctttct
cgccacgttc gccggctttc cccgtcaagc tctaaatcgg
gggctccctt tagggttccg atttagtgct ttacggcacc
togaccccaa aaaacttgat ttgggtgatg gttcacgtag
tgggccatcg ccctgataga cggtttttcg ccctttgacg
ttggagtcca cgttctttaa tagtggactc ttgttccaaa
ctggaacaac actcaaccct atctcgggct attcttttga
tttataaggg attttgccga tttcggccta ttggttaaaa
aatgagctga tttaacaaaa atttaacgcg aattttaaca
aaatattaac gtttacaatt ttatggtgca ctctcagtac
aatctgctct gatgccgcat agttaagcca gccccgacac
ccgccaacac ccgctgacgc gccctgacgg gcttgtctgc
tcccggcatc cgcttacaga caagctgtga ccgtctccgg
gagctgcatg tgtcagaggt tttcaccgtc atcaccgaaa
cgcgcgagac gaaagggcct cgtgatacgc ctatttttat
aggttaatgt catgataata atggtttctt agacgtcagg
tggcactttt cggggaaatg tgcgcggaac ccctatttgt
ttatttttct aaatacattc aaatatgtat ccgctcatga
gacaataacc ctgataaatg cttcaataat attgaaaaag
gaagagtatg agtattcaac atttccgtgt cgcccttatt
cccttttttg cggcattttg ccttcctgtt tttgctcacc
cagaaacgct ggtgaaagta aaagatgctg aagatcagtt
gggtgcacga gtgggttaca tcgaactgga tctcaacagc
ggtaagatcc ttgagagttt tcgccccgaa gaacgttttc
caatgatgag cacttttaaa gttctgctat gtggcgcggt
attatcccgt attgacgccg ggcaagagca actcggtcgc
cgcatacact attctcagaa tgacttggtt gagtactcac
cagtcacaga aaagcatctt acggatggca tgacagtaag
agaattatgc agtgctgcca taaccatgag tgataacact
gcggccaact tacttctgac aacgatcgga ggaccgaagg
agctaaccgc ttttttgcac aacatggggg atcatgtaac
togccttgat cottgggaac cggagctgaa tgaagccata
ccaaacgacg agcgtgacac cacgatgcct gtagcaatgg
caacaacgtt gogcaaacta ttaactggcg aactacttac
tctagcttcc cggcaacaat taatagactg gatggaggcg
gataaagttg caggaccact tctgcgctcg gcccttccgg
ctggctggtt tattgctgat aaatctggag ccggtgagcg
tgggtctcgc ggtatcattg cagcactggg gccagatggt
aagccctccc gtatcgtagt tatctacacg acggggagtc
aggcaactat ggatgaacga aatagacaga tcgctgagat
aggtgcctca ctgattaagc attggtaact gtcagaccaa
gtttactcat atatacttta gattgattta aaacttcatt
tttaatttaa aaggatctag gtgaagatcc tttttgataa
tctcatgacc aaaatccctt aacgtgagtt ttcgttccac
tgagcgtcag accccgtaga aaagatcaaa ggatcttctt
gagatccttt ttttctgcgc gtaatctgct gcttgcaaac
aaaaaaacca ccgctaccag cggtggtttg tttgccggat
caagagctac caactctttt tocgaaggta actggcttca
gcagagcgca gataccaaat actgtccttc tagtgtagcc
gtagttaggc caccacttca agaactctgt agcaccgcct
acatacctcg ctctgctaat cctgttacca gtggctgctg
ccagtggcga taagtcgtgt cttaccgggt tggactcaag
acgatagtta ccggataagg cgcagcggtc gggctgaacg
gggggttcgt gcacacagcc cagcttggag cgaacgacct
acaccgaact gagataccta cagcgtgagc tatgagaaag
cgccacgctt cccgaaggga gaaaggcgga caggtatccg
gtaagcggca gggtcggaac aggagagcgc acgagggagc
ttccaggggg aaacgcctgg tatctttata gtcctgtcgg
gtttcgccac ctctgacttg agcgtcgatt tttgtgatgc
tcgtcagggg ggcggagcct atggaaaaac gccagcaacg
cggccttttt acggttcctg gccttttgct ggccttttgc
tcacatgt (SEQ ID NO:1; TRAC-LHA-pAAV-
U6LbcrTRAC-DR-BbsI-EFS-CD22BBz-TRAC-RHA,
or pXD060).

In SEQ ID NO:1, nucleotides 1-141 correspond to an ITR, nucleotides 156-800 correspond to the TRAC left homology arm, nucleotides 816-1065 correspond to a human U6 promoter, nucleotides 1067-1087 correspond to a direct repeat, nucleotides 1088-1107 correspond to a TRAC targeting crRNA, nucleotides 1108-1128 correspond to a direct repeat, nucleotides 1129-1144 correspond to double BbsI sites, nucleotides 1198-1453 correspond to an EFS-NS promoter, nucleotides 1478-2938 correspond to a CD22BBz CAR, nucleotides 2945-2992 correspond to a polyA signal, nucleotides 2999-3657 correspond to the TRAC right homology arm, and nucleotides 3751-3891 correspond to an rTR.

An exemplary sequence of suitable AAV vector containing an anti-CD19 CAR is provided below:

cctgcaggcagctgcgcgctcgctcgctcactgaggccgcccgggcaaag
cccgggcgtcgggcgacctttggtcgcccggcctcagtgagcgagcgagc
gcgcagagagggagtggccaactccatcactaggggttcctgcggccgca
cgcgtgatgtaaggagctgctgtgacttgctcaaggccttatatcgagta
aacggtagtgctggggcttagacgcaggtgttctgatttatagttcaAAA
CCTCTATCAATGAGAGAGCAATCTCCTGGTAATGTGATAGATTTCCCAAC
TTAATGCCAACATACCATAAACCTCCCATTCTGCTAATGCCCAGCCTAAG
TTGGGGAGACCACTCCAGATTtCAAGATGTACAGTTTGCTTTGCTGGGCC
TTTTTCCCATGCCTGCCTTTACTCTGCCAGAGTTATATTGCTGGGGTTTT
GAAGAAGATCCTATTAAATAAAAGAATAAGCAGTATTATTAAGTAGCCCT
GCATTTCAGGTTTCCTTGAGTGGCAGGCCAGGCCTGGCCGTGAACGTTCA
CTGAAATCATGGCCTCTTGGCCAAGATTGATAGCTTGTGCCTGTCCCTGA
GTCCCAGTCCgTCACGAGCAGCTGGTTTCTAAtATGCTATTTCCCGTATA
AAGCATGAGACCGTGACTTGCCAGCCCCACAGAGCCCCGCCCTTGTCCAT
CACTGGCATCTGGACTCCAGCCTGGGTTGGGGCAAAGAGGGAAATGAGAT
CATGTCCTAACCCTGATCCTCTTGTCCCACAGATATCCAGAACCCTGACC
cgcgttAGCGctagaagagggcctatttcccatgattccttcatatttgc
atatacgatacaaggctgttagagagataattggaattaatttgactgta
aacacaaagatattagtacaaaatacgtgacgtagaaagtaataatttct
tgggtagtttgcagttttaaaattatgttttaaaatggactatcatatgc
ttaccgtaacttgaaagtatttcgatttcttggctttatatatcttGTGG
AAAGGACGAAACACCgTAATTTCTACTAAGTGTAGATGAGTCTCTCAGCT
GGTACACTAATTTCTACTAAGTGTAGATGGTCTTCgaGAAGACCTTTTTT
aagcttggcgtGGATCCGATATCaactagatcttgagacaaggtacctag
gtcttgaaaggagtgggaattggctccggtgcccgtcagtgggcagagcg
cacatcgcccacagtccccgagaaggtaaactgggaaagtgatgtcgtgt
actggctccgcctttttcccgagggtgggggagaaccgtatataagtgca
gtagtcgccgtgaacgttctttttcgcaacgggtttgccgccagaacaca
ggaccggttctagacgtacggccaccatggccttaccagtgaccgccttg
ctcctgccgctggccttgctgctccacgccgccaggccgGATTACAAAGA
CGATGACGATAAGgacatccagatgacacagactacatcctccctgtctg
cctctctgggagacagagtcaccatcagttgcagggcaagtcaggacatt
agtaaGtatttaaattggtatcagcagaaaccagatggaactgttaaact
cctgatctaccatacatcaagattacactcaggagtcccatcaaggttca
gtggcagtgggtctggaacagattattctctcaccattagcaacctggag
caagaagatattgccacttacttttgccaacagggtaatacgcttccgta
cacgttcggaggggggaccaagctggagatcacaggactgcaggagtcag
gacctggcctggtggcgccctcacagagcctgtccgtcacatgcactgtc
tcaggggtctcattacccgactatggtgtaagctggattcgccagcctcc
acgaaagggtctggagtggctgggagtaatatggggtagtgaaaccacat
actataattcagctctcaaatccagactgaccatcatcaaggacaactcc
aagagccaagttttcttaaaaatgaacagtctgcaaactgatgacacagc
catttactactgtgccaaacattattactacggtggtagctatgctatgg
actactggggccaaggaacctcagtcaccgtcAcctcaAccacgacgcca
gcgccgcgaccaccaacaccggcgcccaccatcgcgtcgcagcccctgTc
cctgcgcccagaggcatgccggccagcagcagggggcgcagtgcacacga
gggggctgGacttcgcctgtgatatctacatctgggcgcccttggccggg
acttgtggggtccttctcCtgtcactggttatcaccctttactgcaaacg
gggcagaaagaaactcctgtatatattcAaacaaccatttatgagaccag
tacaaactactcaagaggaagatggctgtagctgccgaTttccagaagaa
gaagaaggaggatgtgaactgagagtgaagttcagcaggagcgcagacGc
ccccgcgtaccagcagggccagaaccagctctataacgagctcaatctag
gacgaagaGaggagtacgatgttttggacaagCgacgtggccgggaccct
gagatggggggaaagccgAgaaggaagaaccctcaggaaggcctgtacaa
tgaactgcagaaagataagatggcggagGcctacagtgagattgggatga
aaggcgagcgccggaggggcaaggggcacgatggccttTaccagggtctc
agtacagccaccaaggacacctacgacgcccttcacatgcaggccctgCc
ccctcgctaaGCTAGCAATAAAAGATCTTTATTTTCATTAGATCTGTGTG
TTGGTTTTTTGTGTggtaccGAGAGACTCTAAATCCAGTGACAAGTCTGT
CTGCCTATTCACCGATTTTGATTCTCAAACAAATGTGTCACAAAGTAAGG
ATTCTGATGTGTATATCACAGACAAAACTGTGCTAGACATGAGGTCTATG
GACTTCAAGAGCAACAGTGCTGTGGCCTGGAGCAACAAATCTGACTTTGC
ATGTGCAAACGCCTTCAACAACAGCATTATTCCAGAAaACACCTTCTTCC
CCAGCCCAGGTAAGGGCAGCTTTGGTGCCTTCGCAGGCTGTTTCCTTGCT
TCAGGAATGGCCAGGTTCTGCCCAGAGCTCTGGTCAATGATGTCTAAAAC
TCCTCTGATTGGTGGTCTCGGCCTTATCCATTGCCACCAAAACCCTCTTT
TTACTAAGAAACAGTGAGCCTTGTTCTGGCAGTCCAGAGAATGACACGGa
AAAAAAGCAGATGAAGAGAAGGTGGCAGGAGAGGGCACGTGGCCCAGCCT
CAGTCTCTCCAACTGAGTTCCTGCCTGCCTGCCTTTGCTCAGACTGTTTG
CCCCTTACTGCTCTTCTAGGCCTCATTCTAAGCCCCTTCTCCAAGTTGCC
TCTCCTTATTTCTCCCTGTCTGCCAAAAAATCTTTCCCAGCTCACTAAGT
CAGTCTCACGCAGTCACTCATTAACCCACAATTCgatatcaagcttAATA
AAAGATCTTTATTTTCATTAGATCTGTGTGTTGGTTTTTTGTGTggtaac
cacgtgcggaccgagcggccgcaggaacccctagtgatggagttggccac
tccctctctgcgcgctcgctcgctcactgaggccgggcgaccaaaggtcg
cccgacgcccgggctttgcccgggcggcctcagtgagcgagcgagcgcgc
agctgcctgcaggggcgcctgatgcggtattttctccttacgcatctgtg
cggtatttcacaccgcatacgtcaaagcaaccatagtacgcgccctgtag
cggcgcattaagcgcggcgggtgtggtggttacgcgcagcgtgaccgcta
cacttgccagcgccctagcgcccgetccagctctaaatcgggggctccct
ttagggttccgatttagtgctttacggcacctcgaccccaaaaaacttga
tttgggtgatggttcacgtagtgggccatcgccctgatagacggtttttc
gccctttgacgttggagtccacgttctttaatagtggactcttgttccaa
actggaacaacactcaaccctatctcgggctattcttttgatttataagg
gattttgccgatttcggcctattggttaaaaaatgagctgatttaacaaa
aatttaacgcgaattttaacaaaatattaacgtttacaattttatggtgc
actctcagtacaatctgctctgatgccgcatagttaagccagccccgaca
cccgccaacacccgctgacgcgccctgacgggcttgtotgctcccggcat
ccgcttacagacaagctgtgaccgtctccgggagctgcatgtgtcagagg
ttttcaccgtcatcaccgaaacgcgcgagacgaaagggcctcgtgatacg
cctatttttataggttaatgtcatgataataatggtttcttagacgtcag
gtggcacttttcggggaaatgtgcgcggaacccctatttgtttatttttc
taaatacattcaaatatgtatccgctcatgagacaataaccctgataaat
gcttcaataatattgaaaaaggaagagtatgagtattcaacatttccgtg
tcgcccttattcccttttttgcggcattttgccttcctgtttttgctcac
ccagaaacgctggtgaaagtaaaagatgctgaagatcagttgggtgcacg
agtgggttacatcgaactggatctcaacagcggtaagatccttgagagtt
ttcgccccgaagaacgttttccaatgatgagcacttttaaagttctgcta
tgtggcgcggtattatcccgtattgacgccgggcaagagcaactcggtcg
ccgcatacactattctcagaatgacttggttgagtactcaccagtcacag
aaaagcatcttacggatggcatgacagtaagagaattatgcagtgctgcc
ataaccatgagtgataacactgcggccaacttacttctgacaacgatcgg
aggaccgaaggagctaaccgcttttttgcacaacatgggggatcatgtaa
ctcgccttgatcgttgggaaccggagctgaatgaagccataccaaacgac
gagcgtgacaccacgatgcctgtagcaatggcaacaacgttgcgcaaact
attaactggcgaactacttactctagcttcccggcaacaattaatagact
ggatggaggcggataaagttgcaggaccacttctgcgctcggcccttccg
gctggctggtttattgctgataaatctggagccggtgagcgtgggtctcg
cggtatcattgcagcactggggccagatggtaagccctcccgtatcgtag
ttatctacacgacggggagtcaggcaactatggatgaacgaaatagacag
atcgctgagataggtgcctcactgattaagcattggtaactgtcagacca
agtttactcatatatactttagattgatttaaaacttcatttttaattta
aaaggatctaggtgaagatcctttttgataatctcatgaccaaaatccct
taacgtgagttttcgttccactgagcgtcagaccccgtagaaaagatcaa
aggatcttcttgagatcctttttttctgcgcgtaatctgctgcttgcaaa
caaaaaaaccaccgctaccagcggtggtttgtttgccggatcaagagcta
ccaactctttttccgaaggtaactggcttcagcagagcgcagataccaaa
tactgtccttctagtgtagccgtagttaggccaccacttcaagaactctg
tagcaccgcctacatacctcgctctgctaatcctgttaccagtggctgct
gccagtggcgataagtcgtgtcttaccgggttggactcaagacgatagtt
accggataaggcgcagcggtcgggctgaacggggggttcgtgcacacagc
ccagcttggagcgaacgacctacaccgaactgagatacctacagcgtgag
ctatgagaaagcgccacgcttcccgaagggagaaaggcggacaggtatcc
ggtaagcggcagggtcggaacaggagagcgcacgagggagcttccagggg
gaaacgcctggtatctttatagtcctgtcgggtttcgccacctctgactt
gagcgtcgatttttgtgatgctcgtcaggggggcggagcctatggaaaaa
cgccagcaacgcggcctttttacggttcctggccttttgctggccttttg
ctcacatgt (SEQ ID NO: 2; TRAC-LHA-pAAV-U6LbcrTRAC-
DR-BbsI-EFS-CD19BBz-TRAC-RHA or pXD071).

The vector sequence of SEQ ID NO:2 generally parallels that of SEQ ID NO:1 with the CD22BBz domain of nucleotides 1478-2938 of SEQ ID NO:1 substituted with the CD19BBz domain. Most particularly, nucleotides 1,478-2,255 of SEQ ID NO:1 encoding the anti-CD22 antigen-binding domain are substituted with an anti-CD19 antigen-binding domain in SEQ ID NO:2.

Although SEQ ID NO:1 and SEQ ID NO:2 contain the sequences for a CD22 CAR and a CD19 CAR, respectively, it is understood that any CAR of interest can be alternatively included, e.g., as illustrated above with SEQ ID NOS:1 and 2. Additionally, these vectors can be modified to contain any guide RNA(s) of interest. For example, the guide RNA targeting the TRAC locus (crTRAC) can be substituted, sequences encoding additional guide RNAs, such as any one of SEQ ID NOs:3-12,134, can be included in the vector (e.g., at the BbsI site), or combinations thereof. Thus, SEQ ID NO:1 or SEQ ID NO:2 are expressly disclosed with or without the sequence encoding the TRAC targeting crRNA, with or without one or more additional crRNA encoding sequences optionally inserted at the BbsI cloning site, and/or the existing CAR encoding sequence or another CAR encoding sequence substituted therefore. In some embodiments, suitable vectors include variants having about 50%, 55%, 60%, 65%, 70%, 75%, 80%, 85%, 90%, 95%, 96%, 97%, 98%, 99%, or more sequence identity to SEQ ID NO:1 or SEQ ID NO:2, or any of the foregoing variations thereof.

The AAV vector used in the compositions and methods can be a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rhlO, AAV.rh32/33, AAV.rh43, AAV.rh64R1, rAAV2-retro, AAV-DJ, AAV-PHP.B, AAV-PHP.S, AAV-PHP.eB, or other natural or engineered versions of AAV. In preferred embodiments, the AAV used in the compositions and methods is AAV6 or AAV9.

Twelve natural serotypes of AAV have thus far been identified, with the best characterized and most commonly used being AAV2. These serotypes differ in their tropism, or the types of cells they infect, making AAV a very useful system for preferentially transducing specific cell types. For example, AAV serotypes 1, 2, 5 or a hybrid capsid AAV1, AAV2, AAV5 or any combination thereof can be used for targeting brain or neuronal cells; AAV4 can be selected for targeting cardiac cells. AAV8 is useful for delivery to the liver cells. Researchers have further refined the tropism of AAV through pseudotyping, or the mixing of a capsid and genome from different viral serotypes. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, AAV2/5 targets neurons that are not efficiently transduced by AAV2/2, and is distributed more widely in the brain, indicating improved transduction efficiency.

Other engineered AAVs have also been developed and can be used for the purpose of introducing transgenes, and in the compositions and methods. These are well known in the art and one of skill in the art would be able to determine the optimal AAV serotype to be used for the respective application.

crRNAs/Guide RNAs

The gene editing compositions include one or more crRNAs (also referred to as guide RNAs) that direct the RNA-guided endonuclease to one or more target genes/sites. Preferably, the crRNAs are provided in the AAV vector (e.g., an AAV6 or AAV9 vector). The crRNAs can be provided individually or together in the form of a crRNA expression cassette. The guide RNA sequence can be configured as a single sequence or as a combination of one or more different sequences, e.g., a multiplex configuration (referred to as an array). For example, in the context of a viral vector, multiple crRNAs/gRNAs can be tandemly arranged, optionally separated by a nucleotide sequence such as a direct repeat in the form of a crRNA expression cassette. The crRNA expression cassette contains one or more regulatory sequences (e.g., U6 promoter) operationally linked to the sequences encoding the crRNAs. For example, the crRNA expression cassette can include multiple gRNAs under the control of a single promoter (e.g., U6 promoter) designed in an array format such that multiple gRNA sequences can be simultaneously expressed. In some embodiments, each individual crRNA or gRNA guide sequence can target a different target. The crRNA expression cassette can encode two or more (e.g., 2, 3, 4, 5, or more) crRNAs that direct the endonuclease to different target genes or target sites (e.g., 2, 3, 4, 5, or more). In preferred embodiments, the crRNA expression cassette encodes two guide RNAs.

The crRNAs/gRNAs can each individually be contained in a composition and introduced to a cell individually or collectively. Alternatively, these components can be provided in a single composition for introduction to a cell. Similarly to the mRNA encoding the RNA-guided endonuclease, the crRNAs or guide RNAs (gRNAs) can be introduced to the cell by any suitable means such as via viral or non-viral techniques. For example, the crRNAs can be provided in a viral vector (e.g., a retrovirus such as a lentivirus, adenovirus, poxvirus, Epstein-Barr virus, adeno-associated virus (AAV), etc.) or by transfection, electroporation or nucleofection for example.

In contrast to Cas9, Cpf1 is tracrRNA independent and requires only an approximately 42 nucleotide long crRNA, which has 20-23 nucleotides at its 3′ end complementary to the protospacer of the target DNA sequence. Cpf1-associated CRISPR arrays are processed into mature crRNAs without the requirement of an additional tracrRNA and when complexed with Cpf1, the Cpf1p-crRNA complex is sufficient to efficiently cleave target DNA by itself. The crRNAs include a spacer sequence (or guide sequence) and a direct repeat sequence. The seed sequence, is approximately within the first 5 nucleotides on the 5′ end of the spacer sequence and mutations within the seed sequence adversely affect cleavage activity of the Cpf1 effector protein complex.

The term “guide RNA,” refers to the polynucleotide sequence containing a putative or identified crRNA sequence or guide sequence. The guide RNA can be any polynucleotide sequence having sufficient complementarity with a target nucleic acid sequence to hybridize with the target nucleic acid sequence and direct sequence-specific binding of an RNA-guided endonuclease to the target nucleic acid sequence. In some embodiments, the degree of complementarity between a guide sequence and its corresponding target sequence, when optimally aligned using a suitable alignment algorithm, is about or more than about 50%, 60%, 70%, 75%, 80%, 85%, 90%, 95%, 97.5%, 99%, or more. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, CA), SOAP (available at soap.genomics.org.cn), and Maq (available at maq.sourceforge.net).

Guide RNA (gRNA) sequences for use in the compositions and methods can be sense or anti-sense sequences. The specific sequence of the gRNA can vary, but, regardless of the sequence, useful guide RNA sequences will be those that minimize off-target effects and achieve high efficiency alteration of the targeted gene or target site. The length of the guide RNA sequence can vary from about 10 to about 60 or more nucleotides, for example about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. The ability of a guide sequence to direct sequence-specific binding of a nucleic acid-targeting complex to a target sequence can be assessed by any suitable assay.

In some embodiments, the crRNA sequence has one or more stem loops or hairpins and is 30 or more nucleotides in length, 40 or more nucleotides in length, or 50 or more nucleotides in length. In certain embodiments, the crRNA sequence is between 42 and 44 nucleotides in length. In some embodiments, the crRNA contains about 19 nucleotides of a direct repeat and between 23 and 25 nucleotides of spacer sequence

In the context of formation of a CRISPR complex, “target sequence” refers to a sequence to which a guide sequence is designed to target, e.g. have complementarity, where hybridization between a target sequence and a guide sequence promotes the formation of a CRISPR complex. The section of the guide sequence through which complementarity to the target sequence is important for cleavage activity is referred to as the seed sequence. A target sequence can comprise any polynucleotide, such as DNA or RNA polynucleotides and is comprised within a target locus of interest.

It is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent to the protospacer (also referred to as target sequence). The skilled person will be able to identify further PAM sequences for use with a given RNA-guided endonuclease. Further, engineering of the PAM Interacting (PI) domain of an RNA-guided endonuclease can allow programing of PAM specificity to improve target site recognition fidelity, and increase the versatility of the Cas, e.g. Cpf1, genome engineering platform. Cas proteins, can be engineered to alter their PAM specificity, for example as described in Kleinstiver, B P., et al., Nature., 523(7561):481-5 (2015).

There are many resources available for helping practitioners determine suitable target sites once a desired DNA target sequence or target gene is identified. For example, numerous public resources, including a bioinformatically generated list of about 190,000 potential guide RNAs, targeting more than 40% of human exons, are available to aid practitioners in selecting target sites and designing the associated guide RNA to affect a nick or double strand break at the site. See also, crispr.u-psud.fr/, a tool designed to help scientists find CRISPR targeting sites in a wide range of species and generate the appropriate crRNA sequences.

The guide RNA can be a sequence complementary to a coding or a non-coding sequence (e.g., a target sequence, target site, or target gene). The gRNA sequences can be complementary to either the sense or anti-sense strands of the target sequences. They can include additional 5′ and/or 3′ sequences that may or may not be complementary to a target sequence. They can have less than 100% complementarity to a target sequence, for example 50%, 60%, 70%, 75%, 80%, 85%, 90%, or 95% complementarity.

Upon formation of a ribonucleoprotein complex with the crRNA, the RNA-guided endonuclease localizes to a sequence (e.g., a target sequence, target site, or target gene) and causes disruption of a target gene and/or one or more homology arms can mediate targeted integration of a transgene at a target site via HDR. A target site can be within the locus of the disrupted gene or at a locus different from the disrupted gene. For example, a target site can overlap with a portion of a gene such as, an enhancer, promoter, intron, exon, or untranslated region (UTR).

Exemplary Target Genes/Target Sites

The gene editing compositions are generally applicable to the targeting and/or alteration (e.g., disruption) of any sequence of interest in the genome, including non-coding and coding regions. One of skill in the art would understand that the targeted sequences would depend on the application for which genome modification is being performed and appropriate crRNAs/gRNAs would be designed accordingly. For example, in the context of CAR T cells, it is desirable to generate standardized therapy in which allogeneic therapeutic cells are administered to a subject in need thereof. By allogeneic is meant that the cells used for treating patients are not originating from said patient but from a donor belonging to the same species, and as such, are genetically dissimilar. However, host versus graft rejection (HvG) and graft versus host disease (GvHD) severely limit their use. In these contexts, it is desirable to generate CAR T cells in which proteins involved in HvG and GvHD have been disrupted. Accordingly, TCR alpha, TCR beta, one or more HLA genes, one or more major histocompatibility complex (MHC) genes, or combinations thereof, can be targeted by the crRNAs/gRNAs.

Immune checkpoints proteins are a group of molecules expressed by T cells that effectively serve as “brakes” to down-modulate or inhibit an immune response. Immune checkpoint molecules include, but are not limited to Programmed Death 1 (PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as HAVCR2, GenBank accession number: JX049979.1), BTLA (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1, SIGLEC10 (GenBank accession number: AY358337.1), 2B4 (also known as CD244, accession number: NM_001 166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, M ORA, IL10RB, HM0X2, IL6R, IL6ST, EIF2AK4, CSK, PAG1, SIT1, F0XP3, PRDM1, BATF, GUCY1A2, GUCY1A3, GUCY1 B2, GUCY1 B3 which directly inhibit immune cells. For example, CTLA-4 is a cell-surface protein expressed on certain CD4 and CD8 T cells; when engaged by its ligands (B7-1 and B7-2) on antigen presenting cells, T-cell activation and effector function are inhibited. Thus, the gene editing compositions can be used to target and inactivate any immune check-point protein, including but not limited to, the aforementioned immune check-point proteins, such as PD1 and/or CTLA-4.

Any gene in the cell's genome can be a target gene or contain a target site. The gene can have a known or putative role in any biological process or molecular function of interest. In some embodiments, a gene with a known or putative role in T cell exhaustion, T cell proliferation, T cell co-stimulation, memory T cell differentiation, T cell receptor signaling, epigenetic regulation, adaptive immune response, immune response to tumor cells, other immune functions, or combinations thereof could be a target gene or target site. Genes involved in such and other biological processes are known and can be determined by one of skill in the art. For example, the Gene Ontology (GO) database and the Molecular Signatures Database (MSigDB) provide lists of genes and/or gene products associated with various biological functions. In some embodiments, a gene listed in Table 2 or Table 3 (provided in Example 1) could be a target gene or target site. In some embodiments, the target gene or target site is a gene or site targeted by one or more guide RNAs selected from the Rene library (SEQ ID NOs:3-4,087) and/or the Descartes library (SEQ ID NOs:4,088-12,134).

In some embodiments, a targeted gene or target site is selected from PRDM1, DPF3, SLAMF1, TE72, HFE, PEL11, PDCD1, HAVCR2/TIM3, TET2, NR4A2, LAIR1, and USB1. In some embodiments, exemplary target genes or target sites include, but are not limited to, PDCD1 and TRAC.

### Chimeric Antigen Receptors (CAR)

Provided as part of the gene editing compositions are one or more CAR expression cassettes containing one or more CARs (e.g., 1, 2, 3, 4, 5, or more) operationally linked to regulatory sequences. Such regulatory sequences can include, without limitation, a promoter, splice acceptor, IRES, 2A peptide, triple helix, polyadenylation signal, or combinations thereof. Upon integration at a target site, the one or more CARs are expressed within the recipient cell (e.g., T cell).

Immunotherapy using T cells genetically engineered to express a chimeric antigen receptor (CAR) is rapidly emerging as a promising new treatment for haematological and non-haematological malignancies. CARs are engineered receptors that possess both antigen-binding and T-cell-activating functions. Based on the location of the CAR in the membrane of the T cell, the CAR can be divided into three main distinct domains, including an extracellular antigen-binding domain, followed by a space region, a transmembrane domain, and the intracellular signaling domain. The antigen-binding moiety, most commonly derived from variable regions of immunoglobulins, is composed of VH and VL chains that are joined up by a linker to form the so-called “scFv.” The segment interposing between the scFv and the transmembrane domain is a “spacer domain,” that in some embodiments, is the constant IgG1 hinge-CH2-CH3 Fc domain. In some cases, the spacer domain and the transmembrane domain are derived from CD8. The intracellular signaling domains mediating T cell activation include a CD3ζ co-receptor signaling domain derived from C-region of the TCR α and β chains and one or more costimulatory domains.

CARs can be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Alternative CAR constructs can be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, costimulation can be orchestrated by expressing CARs in antigen-specific T cells, chosen so as to be activated and expanded following engagement of their native αβTCR, for example by antigen on professional antigen-presenting cells, with attendant costimulation.

In some embodiments, the CAR targets (e.g., recognizes and/or binds) one or more antigens specific for cancer, an inflammatory disease, a neuronal disorder, HIV/AIDS, diabetes, a cardiovascular disease, an infectious disease, an autoimmune disease, or combinations thereof. One of skill in the art, based on general knowledge in the field and/or routine experimentation would be able to determine the appropriate antigen to be targeted by a CAR for a specific disease, disorder or condition.

Exemplary antigens specific for cancer that could be targeted by the CAR include, but are not limited to, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

Exemplary antigens specific for an inflammatory disease that could be targeted by the CAR include, but are not limited to, AOC3 (VAP-1), CAM-3001, CCL11 (eotaxin-1), CD 125, CD 147 (basigin), CD 154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, Lama glama, LFA-1 (CD11a), MEDI-528, myostatin, OX-40, rhuMAb β7, scleroscin, SOST, TGF beta 1, TNF-a, VEGF-A, and combinations thereof.

Exemplary antigens specific for a neuronal disorder that could be targeted by the CAR include, but are not limited to, beta amyloid, MABT5102A, and combinations thereof.

Exemplary antigens specific for diabetes that could be targeted by the CAR include, but are not limited to, L-I β, CD3, and combinations thereof.

Exemplary antigens specific for a cardiovascular disease that could be targeted by the CAR include, but are not limited to, C5, cardiac myosin, CD41 (integrin alpha-lib), fibrin II, beta chain, ITGB2 (CD 18), sphingosine-1-phosphate, and combinations thereof.

Exemplary antigens (or antigen associated viruses) specific for an infectious disease that could be targeted by the CAR include, but are not limited to, anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus, TNFα, and combinations thereof.

In preferred embodiments, the CAR targets one or more antigens selected from AFP, AKAP-4, ALK, Androgen receptor, B7H3, BCMA, Bcr-Abl, BORIS, Carbonic, CD123, CD133, CD44, GD2, Claudins, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6-AML, FAP, Fos-related antigen1, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, lgK, LMP2, MAD-CT-1, MAD-CT-2, MAGE, MelanA/MART1, Mesothelin, MET, ML-IAP, MUC1, mutant p53, MYCN, NA17, NKG2D, NKG2D-L, NY-BR-1, NY-ESO-1, OY-TES1, p53, Page4, PAP, PAX3, PAX5, PD-L1, PDGFR-β, PLAC1, Polysialic acid Proteinase3 (PR1), PSA, PSCA, PSMA, mutant Ras, RGS5, RhoC, ROR1, SART3, sLe(a), Sperm protein 17, SSX2, STn, Survivin, Tie2, Tn, TRP-2, Tyrosinase, VEGFR2, WT1, and XAGE.

Preferably, the CAR can be an anti-CD19 CAR (e.g., CD19BBz) or an anti-CD22 CAR (e.g., CD22BBz). In some embodiments, the CAR can be bispecific. In some embodiments, the CAR can be multivalent. Bispecific or multi-specific (multivalent) CARs, e.g., including, but not limited to, CARs described in WO 2014/4011988 and US20150038684, are contemplated for use in the methods and compositions.

In some embodiments, the CAR expression cassette alternatively or additionally, contains a gene of interest, such as a reporter gene. A reporter gene includes any gene that could be used as an indicator of a successful event, e.g., transfection, transduction, and/or recombination. Reporter genes can be fused to regulatory sequences or genes of interest to report expression location or levels, or serve as controls, for example, standardizing transfection efficiencies. Reporter genes include genes that code for fluorescent protein and enzymes that convert invisible substrates to luminescent or colored products. Reporter genes also include selectable markers that confer the ability to grow in the presence of toxic compounds such as antibiotics or herbicides, which would otherwise kill or compromise the cell. A selectable marker can also confer an ability to utilize a compound, for example, an unusual carbohydrate or amino acid. Non-limiting examples of selectable markers include genes that confer resistance to Blasticidin, G418/Geneticin, Hygromycin B, Puromycin, or Zeocin.

The CAR expression cassettes can each individually be contained in a composition and introduced to a cell individually or collectively. Alternatively, these components can be provided in a single composition for introduction to a cell. Preferably, the one or more CAR expression cassettes are provided in a single viral vector, e.g., an AAV vector packaged in AAV serotypes such as AAV6 or AAV9 vector.

Homology Arms

The gene editing compositions can be used to introduce targeted double-strand breaks (DSB) in an endogenous DNA sequence. The DSB activates cellular DNA repair pathways, which can be harnessed to achieve desired DNA sequence modifications near the break site. In particular embodiments, homologous recombination with one or more homologous sequences is promoted at the site of the DSB, in order to introduce a sequence of interest, such as one or more crRNAs and/or CARs.

In some embodiments, the AAV vector contains one or more homologous sequences (referred to as homology arms) to permit homologous recombination, within or near a target sequence nicked or cleaved by an RNA-guided endonuclease as a part of a nucleic acid-targeting complex. A homology arm can be of any suitable length, such as about or more than about 10, 15, 20, 25, 50, 75, 100, 150, 200, 500, 1000, or more nucleotides in length. In some embodiments, the homology arm is complementary or homologous to a portion of a target sequence. When optimally aligned, a homology arm might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a homology arm and a polynucleotide including a target sequence are optimally aligned, the nearest nucleotide of the homology arm is within about 1, 5, 10, 15, 20, 25, 50, 75, 100, 200, 300, 400, 500, 1000, 5000, 10000, or more nucleotides from the target sequence.

In particular embodiments, the AAV template contains the following components: a 5′ homology arm, a replacement sequence (e.g., crRNA expression cassette and/or CAR expression cassette), and a 3′ homology arm. The homology arms provide for recombination into the chromosome, thus replacing a portion of the endogenous genomic sequence with the replacement sequence. In some embodiments, the homology arms flank the most distal cleavage sites. In some embodiments, the 3′ end of the 5′ homology arm is the position next to the 5′ end of the replacement sequence. In some embodiments, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In some embodiments, the 5′ end of the 3′ homology arm is the position next to the 3′ end of the replacement sequence. In some embodiments, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In some embodiments, the 5′ and 3′ homology arms are homologous to the TRAC locus, for example the first exon of the TRAC locus. Other loci to which the homology arms can be homologous include, but are not limited to, other TCR loci such as TRBC1, TRBC2, TRAV1-1, and TRBV1; immune genes such as PD-1 and B2M; safe harbors such as AAVS1; intergenic regions, and other genomic regions.

In homology-directed repair (HDR), a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site. By a “donor sequence” or “donor polynucleotide” or “donor oligonucleotide” it is meant a nucleic acid sequence to be inserted at the cleavage site (e.g., crRNA expression cassette and/or CAR expression cassette). The donor polynucleotide typically contains sufficient homology to a genomic sequence at the cleavage site, e.g., about 70%, 75%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence includes a non-homologous sequence (e.g., crRNA expression cassette and/or CAR expression cassette) flanked by two regions of homology, such that homology-directed repair between the target DNA region and the two flanking sequences results in insertion of the non-homologous sequence at the target region.

In some embodiments, the sequence containing the one or more homology arms and replacement sequence (referred to hereafter as HDR template) is single stranded or double stranded. In some embodiments, the HDR template is DNA, e.g., double stranded DNA or single stranded DNA. In some embodiments, the HDR template alters the structure of the target position by participating in homologous recombination. In some embodiments, the HDR template alters the sequence of the target position. In some embodiments, the HDR template results in the incorporation of a modified, or non-naturally occurring nucleotide sequence into the target nucleic acid. An HDR template having homology with a target position in a target gene can be used to alter the structure of a target sequence. The HDR template can include sequence which results in: a change in sequence of 1, 2, 3, 4, 5, 6, 7, 8, 9, 10, 11, 12, 13, 14, 15, 20, 30, 40, 50, 60, 70, 80, 90, 100, or more nucleotides of the target sequence.

B. Cells to be Modified and/or Screened

The gene editing compositions and methods can be used to achieve genomic modification and subsequent screening of any cell type. For example, the cell can be a prokaryotic or eukaryotic cell. The cell can be a mammalian cell. The mammalian cell can be human or non-human mammal, e.g., primate, bovine, ovine, porcine, canine, rodent, monkey, rat, or mouse cell. The cell can be a non-mammalian eukaryotic cell such as poultry bird (e.g., chicken), vertebrate fish (e.g., salmon) or shellfish (e.g., oyster, claim, lobster, shrimp) cell. The cell can also be a plant cell.

In preferred embodiments, the cell is a human cell including, but not limited to, skin cells, lung cells, heart cells, kidney cells, pancreatic cells, muscle cells, neuronal cells, human embryonic stem cells, blood cells (e.g., white blood cells), and pluripotent stem cells. More preferably, the cell to be modified can be an immune cell, such as, T cells (e.g., CD8+ T cells such as effector T cells, memory T cells, central memory T cells, and effector memory T cells; or CD4+ T cells such as Th1 cells, Th2 cells, Th3 cells, Th9 cells, Th17 cells, Tfh cells, and Treg cells; or gamma-delta T cells/gdT cells), hematopoietic stem cells (HSC), macrophages, natural killer cells (NK), B cells, dendritic cells (DC), or other immune cells.

In some embodiments, the cell can be from established cell lines, or they can be primary cells, where “primary cells,” refers to cells and cells cultures that have been derived from a subject and allowed to grow in vitro for a limited number of passages or splittings of the culture.

Sources of T Cells

Prior to expansion and genetic modification, the cells (e.g., T cells) can be obtained from a diseased or healthy subject. T cells can be obtained from a number of samples, including peripheral blood mononuclear cells, bone marrow, lymph node tissue, cord blood, thymus tissue, tissue from a site of infection, ascites, pleural effusion, spleen tissue, and tumors. In some embodiments, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media for subsequent processing steps. In some embodiments, the cells are washed with phosphate buffered saline (PBS). The wash solution may lack calcium and/or magnesium or can lack many, if not all, divalent cations. After washing, the cells can be resuspended in a variety of biocompatible buffers, such as, for example, Ca2+-free, Mg2+-free PBS, PlasmaLyte A, or other saline solution with or without buffer. Alternatively, the undesirable components of the apheresis sample can be removed and the cells directly resuspended in culture media.

In some embodiments, T cells can be isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some embodiments, T cells can be isolated by incubation with anti-CD3/anti-CD28-conjugated beads, such as DYNABEADS® M-450 CD3/CD28 T, for a time period sufficient for positive selection of the desired T cells.

C. Pharmaceutical Compositions

Pharmaceutical compositions containing a genetically modified cell or a population of genetically modified cells with a pharmaceutically acceptable buffer, carrier, diluent or excipient are provided. The population of cells can be derived by expanding an isolated genetically modified cell (e.g., a CAR T cell derived using any described components and methods, such as the CLASH system). The cells can be modified to be bispecific or multispecific (e.g., by expressing a bispecific or multispecific CAR, by expressing two or more CARs, etc.). The cell can have been isolated from a diseased or healthy subject prior to genetic modification. Introduction of gene editing compositions (e.g., RNA-guided endonuclease and the one or more AAV vectors) to the cell can be performed ex vivo. In some embodiments, the pharmaceutical compositions contain cells including one or more CARs (e.g., anti-CD19 and/or anti-CD22 CAR) and/or one or more mutations in one or more desired genes, including but not limited to, TCR alpha, TCR beta, HLA genes, histocompatibility complex (MHC) genes, a gene listed in Table 2 or Table 3, TRAC, PRDM1, DPF3, SLAMF1, TET2, HFE, PELI1, PDCD1, HAVCR2/TIM3, TE72, NR4A2, LAIR1, and USB1.

“Pharmaceutically acceptable carrier” describes a pharmaceutically acceptable material, composition, or vehicle that is involved in carrying or transporting a compound of interest from one tissue, organ, or portion of the body to another tissue, organ, or portion of the body. For example, the carrier can be a liquid or solid filler, diluent, excipient, solvent, or encapsulating material, or a combination thereof. Each component of the carrier can be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The compositions can be conveniently formulated into pharmaceutical compositions composed of one or more of the cells in association with a pharmaceutically acceptable carrier. See, e.g., Remington's Pharmaceutical Sciences, latest edition, by E.W. Martin Mack Pub. Co., Easton, PA, which discloses typical carriers and conventional methods of preparing pharmaceutical compositions that can be used. These most typically would be standard carriers for administration of compositions to humans. Such pharmaceutical compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The pharmaceutical compositions can be administered to the subject in a number of ways depending on whether local (e.g., limited to a particular region, physiological system, tissue, organ, or cell type) or systemic treatment is desired, and on the area to be treated. As such, the pharmaceutical compositions can be formulated for delivery via any route of administration. “Route of administration” can refer to any administration pathway known in the art, including but not limited to aerosol, nasal, oral, intravenous, intramuscular, intraperitoneal, inhalation, transmucosal, transdermal, parenteral, transfusion, implantation or transplantation, continuous infusion, topical application, and/or injections.

Parenteral administration, if used, is generally characterized by injection. Injectables can be prepared in conventional forms, either as liquid solutions or suspensions, solid forms suitable for solution or suspension in liquid prior to injection, or as emulsions. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

In some embodiments, the composition is administered to a subject transarterially, subcutaneously, intradermally, intratumorally, intranodally, intramedullarly, intracystically intramuscularly, by intravenous injection, parenterally or intraperitoneally. The composition can be injected directly into a site of inflammation in the subject, a local disease site in the subject, a lymph node, an organ, a tumor, and the like. The pharmaceutical compositions are preferably formulated for intravenous administration. The quantity and frequency of administration will be determined by such factors as the condition of the patient, and the type and severity of the patient's disease, although appropriate dosages can be determined by clinical trials.

### III. Methods of CAR-T Generation

Provided are methods of making a cell or a population of cells (e.g., T cells) that express a chimeric antigen receptor (CAR). CARs are designed in a modular fashion that typically includes an extracellular target-binding domain, a hinge region, a transmembrane domain that anchors the CAR to the cell membrane, and one or more intracellular domains that transmit activation signals. Depending on the number of costimulatory domains, CARs can be classified into first (CD3z only), second (one costimulatory domain+CD3z), or third generation CARs (more than one costimulatory domain+CD3z). Introduction of CAR molecules into a T cell successfully redirects the T cell with additional antigen specificity and provides the necessary signals to drive full T cell activation. Because antigen recognition by CAR T cells is based on the binding of the target-binding single-chain variable fragment (scFv) to intact surface antigens, targeting of cells is not MHC restricted, co-receptor dependent, or dependent on processing and effective presentation of target epitopes.

Typically, CAR T cells are generated by modifying the genome of a recipient T cell to contain and express a CAR. The recipient T cell can be selected from memory T cells, effector T cells, central memory T cells, effector memory T cells, Th1 cells, Th2 cells, Th17 cells, and regulatory T cells. The genome can be edited by an RNA-guided endonuclease, such as Cpf1, that cleaves genomic DNA at a site the RNA-guided endonuclease is directed to by one or more guide RNAs. The vector containing the CAR can have homology arms that facilitate targeted integration of the CAR at the target site (e.g., at or near the site of DNA cleavage). The CAR can be regulated by or expressed under the control of an endogenous or exogenous promoter.

In particular embodiments, a method of making a CAR T cell involves contacting a T cell with an RNA-guided endonuclease and an AAV vector including (i) a crRNA expression cassette encoding a first guide RNA and optionally, a second guide RNA; (ii) a chimeric antigen receptor (CAR) expression cassette; and (iii) 5′ and 3′ homology-directed repair (HDR) arms for targeted genomic integration. The contacting is performed under conditions suitable for genomic editing of the T cell such that the CAR expression cassette is integrated into the genome and subsequently expressed. Suitable conditions can include, without limitation, cell culture and/or other conditions (e.g., media, pH, temperature, CO₂ content, etc.) that allow for the gene editing compositions to be introduced to the cells, expressed, and/or function as needed (e.g., the mRNA encoding the RNA guided endonuclease will be translated so that the endonuclease protein is expressed; the crRNA and/or CAR expression cassettes are integrated into the genome, transcribed and/or translated). In some embodiments, the first guide RNA targets the TRAC locus, the 5′ and 3′ HDR arms are homologous to the TRAC locus, the crRNA expression cassette and CAR expression cassette are integrated into the TRAC locus by HDR, and combinations thereof.

The results of the screens and other assays discussed herein can be used to guide development of other modified cells. For example, genes identified as a important can be knockout or knocked down or otherwise targeted using other means known in the art. Thus, also provided are cells having a heterologous nucleic acid construct encoding a chimeric antigen receptor (CAR) expression cassette and reduced or eliminated expression at one or more gene loci targeted by one or more guide RNAs selected from the group consisting of SEQ ID NOs:3-12,134. These cells need not express the guide RNA. The reduction of expression of the target gene can be modulated by, for example, by (i.e., permanent) genetic mutation or knockout, or by using an inhibitory nucleic acid including but not limited to antisense molecules, siRNA, miRNA, aptamers, ribozymes, triplex forming molecules, RNAi, and external guide sequences, which can be e.g., transiently transfected into cell, or expressed from an expression construct transfected into the cell or integrated into its genome. Such cells can be used in any of the methods, particularly the therapeutic methods, discussed herein.

The following provides exemplary materials and protocols that can be used to generate and characterize CAR T cells.

A. Materials

1. Plasmids & DNA
(i) NSL-LbCpf1-NSL mRNA (TriLink BioTechnologies) Modified mRNA transcript with full substitution of pseudo-U and Capped (Cap 1) using CleanCap™ AG. mRNA can be polyadenylated with DNase and phosphatase treatment. mRNA can be purified by silica membrane and packaged as a solution in 1 mM Sodium Citrate, pH 6.4.
(ii) Plasmids: AAV6/AAV9, PDF6, AAV vector including pXD060, pXD017, pXD071, or derivatives for any of those vectors, with or without crRNAs chosen from the Rene library, Descartes library, or other crRNA.

2. Cell Lines
(i) Human peripheral blood CD8+ and/or CD4+ T cells (STEMCELL Technologies, or other donors)
(ii) HEK293FT cells (ThermoFisher)
(iii) NALM6 cells (ATCC)

3. Kits & Chemicals

### Antibodies and Staining Reagents:

APC anti-human TIGIT—Biolegend; Catalog Number: 372705
APC/Cyanine7 anti-human CD8a—Biolegend; Catalog Number: 300926
FITC anti-human CD197 (CCR7) Antibody—Biolegend; Catalog Number: 353216
FITC anti-human CD3 Antibody—Biolegend; Catalog Number: 300306
PE anti-human IgG Fc—Biolegend; Catalog Number: 409304
Brilliant Violet 510™ anti-human CD8—Biolegend; Catalog Number: 344732
Brilliant Violet 421™ anti-human CD62L—Biolegend; Catalog Number: 304828
PerCP/Cyanine5.5 anti-human/mouse Granzyme B—Biolegend; Catalog Number: 372212
Brilliant Violet 421™ anti-human CD366 (Tim-3)—Biolegend; Catalog Number: 345008
FITC anti-human TNF—Biolegend; Catalog Number: 502906
APC anti-human CD25—Biolegend; Catalog Number: 356109
PerCP/Cyanine5.5 anti-human CD27—Biolegend; Catalog Number: 393209
PE anti-DYKDDDDK (SEQ ID NO:12,246) Tag—Biolegend; Catalog Number: 637310
PE/Cy7 anti-human CD197 (CCR7) Antibody—Biolegend; Catalog Number: 353225
APC anti-human CD45RO—Biolegend; Catalog Number: 304210
APC anti-human IFN—Biolegend; Catalog Number: 506510
PerCP/Cyanine5.5 anti-human CD223 (LAG-3)—Biolegend; Catalog Number: 369312
APC anti-human CD127 (IL-7Ra) [Clone: A019D5]—Biolegend; Catalog Number: 351315
Brilliant Violet 421™ anti-human CD210 (IL-10 R)—Biolegend; Catalog Number: 308815
PerCP/Cyanine5.5 anti-human CD122 (IL-2Rβ)—Biolegend; Catalog Number: 339011
PE/Cyanine7 anti-human CD244 (2B4)—Biolegend; Catalog Number: 329519
APC/Cyanine7 anti-human CD294 (CRTH2)—Biolegend; Catalog Number: 350113
APC/Cyanine7 anti-human CD28—Biolegend; Catalog Number: 302965
FITC anti-human CD69 [Clone: FN50]— Biolegend; Catalog Number: 310903
Human Monoclonal BLIMP1/PRDM1 Antibody—R&D; Catalog Number: MAB36081
Blimp-1/PRDI-BF1 (C14A4) Rabbit mAb—CST; Catalog Number: 9115
Recombinant Human Siglec-2/CD22 Fc Chimera Protein—R&D; Catalog Number: 1968-SL-050
Pierce™ Recombinant Biotinylated Protein L—ThermoFisher; Catalog Number: 21189

### Bacterial and Virus Strains:

One Shot Stb13 Chemical Competent E. coli—ThermoFisher; Catalog Number: C737303
Endura™ ElectroCompetent Cells—Lucigen; Catalog Number: 60242-2
   qPCR Probes:

| | |
|---|---|
| ID2 | ThermoFisher Assay ID: Hs04187239_m1 |
| RASA3 | ThermoFisher Assay ID: Hs01071043_m1 |
| PCDH8 | ThermoFisher Assay ID: Hs00159910_ml |
| IFIT3 | ThermoFisher Assay ID: Hs01922752_s1 |
| TNFSF4 | ThermoFisher Assay ID: Hs00967195_m1 |
| RIN3 | ThermoFisher Assay ID: Hs01112081_m1 |
| PTPN14 | ThermoFisher Assay ID: Hs00193643_m1 |
| CDCA7 | ThermoFisher Assay ID: Hs00230589_m1 |
| RUNX3 | ThermoFisher Assay ID: Hs00231709_m1 |
| FOXO1 | ThermoFisher Assay ID: Hs01054576_m1 |
| TBX21 | ThermoFisher Assay ID: Hs00203436_m1 |
| BATF | ThermoFisher Assay ID: Hs00232390_m1 |
| CXCR6 | ThermoFisher Assay ID: Hs00174843_m1 |
| PRF1 | ThermoFisher Assay ID: Hs00169473_m1 |
| STAT6 | ThermoFisher Assay ID: Hs00598625_m1 |
| STAT1 | ThermoFisher Assay ID: Hs01013996_m1 |
| SOCS1 | ThermoFisher Assay ID: Hs00705164_s1 |
| IL13 | ThermoFisher Assay ID: Hs00174379_m1 |
| WNT11 | ThermoFisher Assay ID: Hs00182986_m1 |
| KLF2 | ThermoFisher Assay ID: Hs00360439_g1 |
| S1PR1 | ThermoFisher Assay ID: Hs00173499_m1 |
| IRF4 | ThermoFisher Assay ID: Hs01056534_m1 |
| NFKB1 | ThermoFisher Assay ID: Hs00765730_m1 |
| GAPDH | ThermoFisher Assay ID: Hs02786624_g1 |
| PRDM1_ISO1 | ThermoFisher Assay ID: APU6667 |
| PRDM1_ISO2 | ThermoFisher Assay ID: APRWH2D |
| PRDM1_ISO3 | ThermoFisher Assay ID: APT2DMA |

### Chemicals, Peptides, and Recombinant Proteins:

DPBS, no calcium, no magnesium—Gibco; Catalog Number: 14190250
RPMI 1640 Medium—Gibco; Catalog Number: 11875-093
DMEM, high glucose, pyruvate—Gibco; Catalog Number: 11995065
Fetal Bovine Serum—Sigma Aldrich; Catalog Number: F4135-500ML
Penicillin-Streptomycin (10,000 U/mL)—Gibco; Catalog Number: 15140122
2-Mercaptoethanol—Sigma Aldrich; Catalog Number: M6250-10ML
X-VIVO 15 Serum-free Hematopoietic Cell Medium—Lonza; Catalog Number: BE02-060F
Corning; Human AB Serum; Male Donors; type AB; US; 100 mL, 35-060-CI 1/EA—Corning; Catalog Number: MT35060CI
ACK Lysing Buffer—Lonza; Catalog Number: 10-548E
PEI MAX—Polyscience; Catalog Number: 24765-1
PEG8000—Promega; Catalog Number: V3011
RIPA buffer—Boston BioProducts; Catalog Number: BP-115
protease inhibitor coctail—ThermoFisher, Catalog Number: 78437
Pierce™ BCA Protein Assay Kit—ThermoFisher, Catalog Number: 23227
LS Columns—Miltenyi; Catalog Number: 130-042-401
Human CD8 T Cell Isolation Kit—Miltenyi; Catalog Number: 130-096-495
streptavidin microbeads—Miltenyi; Catalog Number: 130-048-102
FcR Blocking Reagent, Human—Miltenyi; Catalog Number: 130-059-901
Pierce™ NHS-Activated Agarose Slurry—ThermoFisher; Catalog Number: 26200
Recombinant Human IL-2 (carrier-free)—Biolegend; Catalog Number: 589104
BD Cytofix/Cytoperm™ Fixation/Permeabilization Solution Kit—BD; Catalog Number: 554714
QuickExtract DNA Extraction Solution—Epicenter; Catalog Number: QE09050
QIAamp DNA Blood Mini Kit—Qiagen; Catalog Number: 51106
T7 Endonuclease I—NEB; Catalog Number: M0302L
Proteinase K—Qiagen; Catalog Number: 19131
RNAse A—Qiagen; Catalog Number: 19101
Gibson Assembly® Master Mix—NEB; Catalog Number: E2611
Phusion Flash High-Fidelity PCR Master Mix—ThermoFisher, Catalog Number: F548L
DreamTaq Green PCR Master Mix (2×)—ThermoFisher, Catalog Number: K1082
QIAquick Gel Extraction Kit QiagenCatalog Number: 28706
E-Gel™ Low Range Quantitative DNA Ladder—ThermoFisher, Catalog Number: 12373031
NEBNext® Ultra™ RNA Library Prep Kit—NEB; Catalog Number: E7530S
NEBNext® Multiplex Oligos for illumina—NEB; Catalog Number: E7335S
Nextera DNA Library Prep Kit—Illumina; Catalog Number: FC-121-1030
Nextera Index Kit—Illumina; Catalog Number: FC-121-1011
TRIzol™ Reagent—Invitrogen; Catalog Number: 15596026
RNeasy Plus mini isolation kit—Qiagen; Catalog Number: 74134
M-MLV reverse transcriptase enzyme—Sigma Aldrich; Catalog Number: M1302-40KU
Oligo(dT)20 Primer—ThermoFisher, Catalog Number: 18418020
TaqMan® Fast Universal PCR Master Mix—Invitrogen; Catalog Number: 4352042
BpiI (BbsI) (10 U/μL)—ThermoFisher; Catalog Number: ER1012
Benzonase® Nuclease—ThermoFisher, Catalog Number: E1014-25KU
4-20% Mini-PROTEAN® TGX™ Precast Protein Gels, 10-well—BioRad; Catalog Number: 4561094
Bovine Serum Albumin—Sigma Aldrich; Catalog Number: A9418-100G
Pierce™ ECL Western Blotting Substrate—ThermoFisher, Catalog Number: 32106
EDTA—Sigma Aldrich; Catalog Number: E8008-100ML
XenoLight D-Luciferin—K+Salt Bioluminescent Substrate—Perkin Elmer; Catalog Number: 122799
Neon™ Transfection System 100 μL Kit—Invitrogen; Catalog Number: MPK10025.

B. Equipment
(i) PCR Thermocycler
(ii) Tissue culture hood
(iii) 15-cm tissue culture dishes (Corning)
(iv) Retronectin-coated plates (Takara)
(v) Neon® Transfection System (ThermoFisher)
(vi) Bioanalyzer (Agilent)
(vii) Pipettes and tips
(viii) Next generation sequencing machines (Illumina)
(ix) Cell culture incubators (37° C., 5% CO₂)
(x) Countess automated cell counter (Thermo Fisher)
(xi) Plate reader (PerkinElmer)
(xii) BD FACSAria II (BD Biosciences)
(xiii) FlowJo software 9.9.4 (Treestar, Ashland, OR)

C. Construction of AAV Vectors

1. crRNA Expression Vector Design and Construction
(i) Identify genes for knockout by targeted delivery of HDR template. TRAC is used as an example, but any gene with a Cpf1 PAM sequence can be targeted.
(ii) Design LbCpf1 crRNA (20 bp) with Benchling or other computational pipelines. crTRAC: GAGTCTCTCAGCTGGTACAC (SEQ ID NO:12,135)
(iii) Synthesize oligonucleotides with two LbCpf1 direct repeats and sticky ends.
(iv) Digest pXD060, pXD017, or pXD071 with FD BbsI and insert guide after U6 promoter.

2. CAR Sequence Generation
(i) Generation of CD22BBz CAR can be performed as previously described (Haso, W., et al., Blood., 121(7):1165-74 (2013). CD22 binding scFV (m971) specific for the human CD22 followed by CD8 hinge-transmembrane-regions linked to 4-1BB (CD137) intracellular domains and CD3ζ intracellular domain.
(ii) The sequence of CD19 binding scFv (FMC63) can be found from NCBI (GenBank: HM852952) and can be followed by CD8 hinge-transmembrane-regions linked to 4-1BB (CD137) intracellular domains and CD3ζ intracellular domain (Kochenderfer, J N., et al., J. Immunother., 32(7):689-702 (2009)). In order to detect CD19BBz CAR in different way, the Flag or other tag sequence can be added after the CD8a leader sequence.
(iii) Synthesize m971-BBz and FMC63-BBz using gBlock (IDT).

3. HDR Template Design
(i) Amplify left and right homologous arms of the TRAC locus from primary CD4+ T cells by PCR using locus-specific primer sets with multiple cloning site (MCS). PCR annealing temperature (60° C.).
(ii) Sequence amplicons

4. AAV-crRNA-HDR-CAR Vector Cloning
(i) Clone HDR sequences into the AAV vector (pXD060) by Gibson assembly. Incubate samples in a thermocycler at 50° C. for 30 minutes.
(ii) pXD071 (CD19CAR) construction: Digest pXD040 and then clone CAR sequences into MCS by Gibson assembly.

D. AAV Production and Titration

1. AAV Production
(i) Transfect HEK293FT cells with AAV constructs in 15-cm tissue culture dishes, AAV2 transgene vectors, packaging (pDF6) plasmid, and AAV6/9 serotype plasmid together with polyethyleneimine (PEI).
(ii) Collect transfected cells with PBS after 72 hours of transfection.

2. AAV Purification and Titration
(i) Mix transfected cells with pure chloroform ( 1/10 volume).
(ii) Incubate cells at 37° C. with vigorous shaking for 1 hour.
(iii) Add NaCl to a final concentration of 1 M.
(iv) Centrifuge at 20,000 g at 4° C. for 15 minutes.
(v) Transfer aqueous layer to another tube and discard the chloroform layer.
(vi) Add PEG8000 to the sample until 10% (w/v) and shake until dissolved.
(vii) Incubate the mixture at 4° C. for 1 hour and then centrifuge at 20,000 g at 4° C.
(viii) Discard supernatant and suspend the pellet in DPBS with MgCl₂.
(ix) Treat the sample with universal nuclease and incubate at 37° C. for 30 minutes.
(x) Add chloroform (1:1 volume), shake and centrifuge at 12,000 g at 4° C. for 15 minutes.
(xi) Isolate the aqueous layer and concentrate through a 100-kDa MWCO. Concentrate AAV at high concentration so the volume can be reduced when performing the infection, which can decrease the toxicity of AAV. AAV should be aliquoted and stored at −80° C.
(xii) Titer virus by qPCR using custom Taqman assays (ThermoFisher) targeted to promoter U6.

E. T Cell Electroporation

Human primary peripheral blood CD4+ T cells can be acquired from healthy donors (STEMCELL technologies). T cells can be cultured in X-VIVO media (Lonza) with 5% human AB serum and recombinant human IL-2 30 U/mL.
(i) Activate T cells with CD3/CD28 Dynabeads for 2 days prior to electroporation.
(ii) Use magnetic holder to remove Dynabeads.
(iii) Prepare cells at a density of 2×10⁵ cells per 10 μL tip reaction or 2×10⁶ cells per 100 μL tip reaction in electroporation Buffer R (Neon Transfection System Kits).
(iv) Mix with 1 μg or 10 μg of modified NLS-LbCpf1-NLS mRNA (TriLink) according to reaction volume.
(v) Electric shock at program 24 (1,600V, 10 ms and three pulses).
(vi) Transfer cells into 200 μl or 1 mL of pre-warmed X-VIVO media (without antibiotics) immediately after electroporation.
(vii) Add indicated volumes of AAV (AAV volume to not exceed 20% of culture volume) into the T cells 2-4 hours after electroporation. The CAR(s) will begin to be expressed after two to three days and have enrichment after stimulation with target cells.

F. CAR-T Detection by Flow Cytometry
(i) After electroporation for 5 days, incubate 1×10⁶ CD22BBz CAR transduced T cells with 0.2 μg CD22-Fc (R&D system) in 100 μL PBS for 30 minutes, and then stain with PE-IgG-Fc and FITC-CD3 antibodies for 30 minutes.
(ii) For CD19CAR detection, incubate CD19BBz CAR transduced T cells with APC-anti-Flag and FITC-CD3 antibodies for 30 minutes.
(iii) Wash cells twice and quantify and sort labeled cells on BD FACSAria II.
(iv) The staining patterns can be analyzed using FlowJo software 9.9.4 (Treestar, Ashland, OR).

G. T7E1 Assay

Five days after electroporation, harvest the bulk transduced T cells and sorted T cells. The genomic DNA can be collected using the QuickExtract DNA Extraction Solution (Epicentre).
(i) PCR amplify target loci from genomic DNA around cut site.
(ii) Run PCR amplicons on 2% E-gel EX and purify (with known band size) using QIAquick Gel Extraction Kit.
(iii) After purification, denature 200 ng of purified PCR product, anneal, and digest with T7E1, 37° C. 45 minutes (New England BioLabs).
(iv) Load digested PCR products into 2% E-gel EX and quantify DNA fragment abundance using E-Gel™ Low Range Quantitative DNA Ladder (ThermoFisher).

H. HDR Quantification and NGS Sequencing Analysis

1. Semi-Quantitative In-Out PCR
(i) Use three primers for In-Out PCR:
   TRAC 1st: binds to a sequence of the left TRAC homology arm
   TRAC 2nd: binds to genomic sequence outside of this AAV donor
   CD22CAR 3rd primer: recognizes a sequence contained in the m971-BBz cassette
(ii) Normalize amplicon (labeled TRAC-HDR) concentration by comparison to the product resulting from the uninfected control with genomic DNA isolated from human CD4+ T cells.
(iii) PCR products can be used for Nextera library preparation following the manufacturer's protocols (e.g., Illumina).
(iv) Prepped libraries can be sequenced on 100-bp single-end reads on an Illumina HiSeq 4000 instrument or equivalent.

2. Indel Quantification
(i) Some PCR products from amplification around cut site of genomic DNA (same samples as T7E1 assay) can be used for Nextera library preparation following the manufacturer's protocols (Illumina).
(ii) Prepped libraries can be sequenced on 100-bp paired-end reads on an Illumina HiSeq 4000 instrument or equivalent (generating 29 to 74 million reads per library).
(iii) Map paired reads to amplicon sequences (expected sequences provided in FASTA form to generate indices) using BWA-MEM with the −M option.
(iv) Discard 100 bp reads in SAM file that fall outside a +/−75 bp window of expected cut site within the amplicon.
(v) Discard soft-clipped reads (identified with “S” character in CIGAR string).
(vi) Identify indel reads by the presence of “I” or “D” characters within the CIGAR string.
(vii) Quantify cutting efficiency as percentage of indels over total (indel plus wild-type reads) within the defined window.

I. Co-culture Functional Assays

1. Stable Cell Line Generation
(i) Generate lentivirus including GFP-Luciferase reporter genes.
(ii) Infect NALM6 cells (ATCC) with 2× concentrated lentivirus by spinoculation in retronectin-coated (Takara) plates at 800 g for 45 minutes at 32° C.
(iii) After infection for 2 days, sort GFP positive cells (NALM6-GL) by flow cytometry.
(iv) Perform a second round of sorting after culturing for an additional two days.
(v) Incubate cells with 150 μg/ml D-Luciferin (PerkinElmer) and measure bioluminescence signal intensity by an IVIS system to assess luciferase expression.

2. Cancer Cell Cytolytic Assay (Kill Assay)
(i) Seed 2×10⁴ NALM6-GL cells in a 96 well plate.
(ii) Co-culture modified T cells with NALM6-GL at indicated E:T ratios for 24 hours.
(iii) Add 150 μg/ml D-Luciferin (PerkinElmer) into each well and measure luciferase assay intensity by a plate reader (PerkinElmer) to assess cell proliferation.

3. T Cell Exhaustion Assay
(i) Co-culture CAR T cells with NALM6-GL cells at appropriate E:T ratio (e.g., 0.2:1) for appropriate time period (e.g., 24 hours).
(ii) Collect cells and wash once by DPBS. Incubate cells with 0.2 μg CD22-Fc (R&D Systems) in 100 μL DPBS for 30 minutes.
(iii) Stain cells with PE-IgG-Fc, PD-1-FITC, TIGIT-APC and LAG3-Percp/cy5.5 (Biolegend) for 30 minutes.
(iv) Measure stained cells by flow cytometry.

4. Intracellular staining of IFNγ and TNF-α
(i) After infection for 5 days, co-culture AAV transduced CD22BBz CAR-T cells with NALM6 at 1:1 E:T ratio in fresh media supplemented with brefeldin A and 2 ng/mL IL-2.
(ii) After 5 hours of incubation, collect and stain for surface CAR.
(iii) Fix and permeabilize cells by fixation/permeabilization solution (BD) and add anti-IFNγ-APC or anti-TNF-α-FITC for intracellular staining.
(iv) After 30 minutes, wash stained cells by BD Perm/Wash™ buffer and measure cells by flow cytometry.

### IV. Methods of Use

A. Screens

Methods of performing screens are provided. Typically, the screens are designed to identify genes involved in one or more phenotypes of interest. Exemplary cellular phenotypes include increased tumor/tumor microenvironment infiltration, increased target cell affinity, increased target cell cytotoxicity, increased persistence, increased expansion/proliferation, reduced exhaustion, increased anti-cancer metabolic function, increased ability to prevent immune escape, reduced unspecific cytokine production, reduced off-target toxicity, reduced cytokine release syndrome (CRS) (e.g., when introduced in vivo), and combinations thereof. The screens can be loss of function or gain of function. The screens can be performed in vitro (e.g., in cultured cells) or in vivo (e.g., in a subject such as a mouse or rat).

Typically, the screen involves contacting cells with a library of vectors containing guide RNAs and/or CARs and an RNA-guided endonuclease (e.g., Cpf1). The screen is performed under conditions that permit the cells to undergo genetic modification (e.g., knock-in and subsequent expression of a CAR and/or alteration of a target gene or target site). The screen method can further include applying selective pressure to the cells in order to enrich for cells that exhibit a desired phenotype. In some embodiments, the method can include identifying guide RNAs that are enriched or highly represented (e.g., compared to control guide RNAs) in the cells that have been selected. In some embodiments, the method includes identifying guide RNAs that are depleted or under-represented (e.g., compared to control guide RNAs) in the cells that have been selected. The enrichment or depletion of guide RNAs can be relative to a time point before selection (e.g., day 0), non-targeting guide RNAs, or combinations thereof. Since the gene targeted by each guide RNA is known, identification of the guide RNAs allows for identification of the genes contributing to the phenotype of interest. Results of the screen can be validated by independently generating cells containing one or more modifications (e.g., mutations) in the one or more genes identified by the screen. The same or different guide RNAs may be used for validation.

An exemplary screen for identifying one or more genes that enhance a desired phenotype of a cell containing a CAR includes (a) contacting a population of cells with an RNA-guided endonuclease and a library containing a plurality of vectors, wherein each vector independently contains (i) a crRNA expression cassette encoding a first guide RNA and a second guide RNA; (ii) a CAR expression cassette; and (iii) 5′ and 3′ homology arms for targeted genomic integration via homology directed repair (HDR); and (b) selecting for cells exhibiting the desired phenotype. In some preferred embodiments, each AAV vector in the plurality of AAV vectors contains a unique second guide RNA. The contacting is performed under conditions that allow targeted genomic integration of the crRNA and CAR expression cassettes and expression of the guide RNAs and CAR encoded therein. In some embodiments, the first guide RNA targets the TRAC locus; the second guide RNA targets a gene involved in T cell exhaustion, T cell proliferation, T cell co-stimulation, memory T cell differentiation, T cell receptor signaling, epigenetic regulation, adaptive immune response, immune response to tumor cells, and/or other immune functions; or a combination thereof. In some embodiments, the first guide RNA targets the TRAC locus and/or the second guide RNAs within the population of cell collectively target any gene in the genome, such as one or more genes selected from Table 2 or Table 3.

The method can further include identifying the crRNA expression cassette present in the cells that have been selected such that the genes that enhance the desired phenotype are identified based on their targeting by the guide RNAs encoded by the crRNA expression cassette. In some embodiments, identification of the crRNA expression cassette can be achieved by sequencing genomic DNA.

B. Methods of Treatment

Methods of inducing or increasing an immune response in a subject by administering to the subject an effective amount of a pharmaceutical composition containing a population of genetically modified cells (e.g., CAR T cells) are provided.

The agents and compositions can also be used in methods of treating a disease, disorder, or condition. An exemplary method involves treating a subject (e.g., a human) having a disease, disorder, or condition by administering to the subject an effective amount of a pharmaceutical composition containing a population of genetically modified cells (e.g., CAR T cells). In some embodiments, the disease, disorder, or condition is associated with an elevated expression or specific expression of an antigen. In some embodiments, the cells administered to the subject contain/express a CAR that targets the antigen.

In some embodiments, the cell(s) are isolated from the subject having the disease, disorder, or condition, or from a healthy donor, prior to genetic modification. For example, in some embodiments, the method of treatment involves (i) obtaining cells from a subject (e.g., T cells), (ii) modifying the cells to express a heterologous CAR, and (iii) administering an effective amount of the modified cells to the subject. In some embodiments, the CAR recognizes an antigen associated with the disease, disorder, or condition. Any of the methods of treatment can further include expanding the population of cells before and/or after undergoing genetic modification.

In some embodiments, besides integration and expression of a CAR, the cell is further modified by one or more mutations causing reduced function or loss of function of one or more genes (or gene products thereof) selected from PRDM1, DPF3, SLAMF1, TET2, HFE, PELI1, PDCD1, HAVCR2/TIM3, TET2, NR4A2, LAIR1, USB1, and genes listed in Table 2 or Table 3.

Diseases to be Treated

The subject administered the compositions can have a disease, disorder, or condition such, as but not limited to, cancer, an inflammatory disease, a neuronal disorder, HIV/AIDS, diabetes, a cardiovascular disease, an infectious disease, an immune system disorder such autoimmune disease, or combinations thereof.

1. Cancers

Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell., 144:646-674, (2011)).

Tumors which can be treated in accordance with the methods are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The compositions and methods are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth.

In some embodiments, the cancer is a liquid cancer (e.g., acute myeloid leukemia (AML), chronic lymphocytic leukemia (CLL), mantle cell lymphoma (MCL), multiple myeloma (MM), acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), chronic myeloid leukemia, myeloproliferative neoplasms, follicular lymphoma, myelodysplasia, myelodysplastic syndrome, non-Hodgkin lymphoma, plasmablastic lymphoma, plasmacytoid dendritic cell neoplasm, and Waldenstrom macroglobulinemia).

In some embodiments, the cancer is a solid cancer. The term “solid cancer” refers to an abnormal mass of tissue that usually does not contain cysts or liquid areas. Solid cancers may be benign or malignant. Different types of solid cancers are named for the type of cells that form them. Examples of solid cancers include but are not limited to, mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

TABLE 1
Provides a non-limiting list of cancers for which the CAR of the methods
and compositions can target a specific or an associated antigen.

| | | | | |
|---|---|---|---|---|
| Acute Lymphoblastic | Acute Myeloid Leukemia | Adrenocortical | AIDS-Related Cancers | Kaposi Sarcoma |
| Leukemia (ALL) | (AML) | Carcinoma | | |
| AIDS-Related | Primary CNS Lymphoma | Anal Cancer | Appendix Cancer | Astrocytomas |
| Lymphoma | | | (Gastrointestinal | |
| | | | Carcinoid Tumors) | |
| Atypical Teratoid/ | Brain Cancer | Basal Cell Carcinoma | Bile Duct Cancer | Bladder Cancer |
| Rhabdoid Tumor | | of the Skin | | |
| Bone Cancer (includes | Brain Tumors | Breast Cancer | Bronchial Tumors | Burkitt Lymphoma |
| Ewing Sarcoma and | | | | |
| Osteosarcoma and | | | | |
| Malignant Fibrous | | | | |
| Histiocytoma) | | | | |
| Non-Hodgkin | Carcinoid Tumors | Carcinoma of | Cardiac (Heart) Tumors | Embryonal Tumors |
| Lymphoma | | Unknown Primary | | |
| Germ Cell Tumor | Primary CNS Lymphoma | Cervical Cancer | Cholangio-carcinoma | Chordoma |
| Chronic Lymphocytic | Chronic Myelogenous | Chronic | Colorectal Cancer | Cranio-pharyngioma |
| Leukemia (CLL) | Leukemia (CML) | Myeloproliferative | | |
| | | Neoplasms | | |
| Cutaneous T-Cell | Ductal Carcinoma In Situ | Endometrial Cancer | Ependymoma | Esophageal Cancer |
| Lymphoma (Mycosis | (DCIS) | | | |
| Fungoides and Sézary | | | | |
| Syndrome) | | | | |
| Esthesioneuro-blastoma | Ewing Sarcoma | Extracranial Germ Cell | Eye Cancer | Intraocular |
| | | Tumor | | Melanoma |
| Fallopian Tube Cancer | Fibrous Histiocytoma of | Osteosarcoma | Gallbladder Cancer | Gastric Cancer |
| | Bone | | | |
| Stomach Cancer | Gastrointestinal Carcinoid | Gastrointestinal | Central Nervous | Extracranial Germ |
| | Tumor | Stromal Tumors | System Germ Cell | Cell Tumors |
| | | (GIST) | Tumors | |
| Extragonadal Germ Cell | Ovarian Germ Cell | Testicular Cancer | Gestational | Hairy Cell Leukemia |
| Tumors | Tumors | | Trophoblastic Disease | |
| Head and Neck Cancer | Heart Tumors | Hepatocellular (Liver) | Histiocytosis | Hodgkin Lymphoma |
| | | Cancer | (Langerhans Cell) | |
| Hypopharyngeal Cancer | Intraocular Melanoma | Islet Cell Tumors | Pancreatic | Kidney Cancer |
| | | | Neuroendocrine | |
| | | | Tumors | |
| Renal Cell Cancer | Langerhans Cell | Laryngeal Cancer | Leukemia | Lip and Oral Cavity |
| | Histiocytosis | | | Cancer |
| Liver Cancer | Lung Cancer (Non-Small | Lymphoma | Male Breast Cancer | Malignant Fibrous |
| | Cell and Small Cell) | | | Histiocytoma of |
| | | | | Bone and |
| | | | | Osteosarcoma |
| Melanoma | Intraocular (Eye) | Merkel Cell Carcinoma | Malignant | Metastatic Cancer |
| | Melanoma | (Skin Cancer) | Mesothelioma | |
| Metastatic Squamous | Midline Tract Carcinoma | Mouth Cancer | Multiple Endocrine | Multiple |
| Neck Cancer with | With NUT Gene Changes | | Neoplasia Syndromes | Myeloma/Plasma |
| Occult Primary | | | | Cell Neoplasms |
| Mycosis Fungoides | Myelodysplastic | Myelodysplastic/ | Nasal Cavity and | Nasopharyngeal |
| (Lymphoma) | Syndromes | Myeloproliferative | Paranasal Sinus Cancer | Cancer |
| | | Neoplasms | | |
| Neuroblastoma | Non-Small Cell Lung | Oral Cancer | and Oropharyngeal | Ovarian Cancer |
| | Cancer | | Cancer | |
| Pancreatic Cancer | Papillomatosis | Paraganglioma | Paranasal Sinus and | Parathyroid Cancer |
| | | | Nasal Cavity Cancer | |
| Penile Cancer | Pharyngeal Cancer | Pheochromocytoma | Pituitary Tumor | Plasma Cell |
| | | | | Neoplasm/Multiple |
| | | | | Myeloma |
| Pleuropulmonary | Primary Central Nervous | Primary Peritoneal | Prostate Cancer | Rectal Cancer |
| Blastoma | System (CNS) Lymphoma | Cancer | | |
| Recurrent Cancer | Retinoblastoma | Rhabdomyosarcoma | Salivary Gland Cancer | Sarcoma |
| Vascular Tumors | Uterine Sarcoma | Sézary Syndrome | Small Cell Lung Cancer | Small Intestine |
| | | (Lymphoma) | | Cancer |
| Soft Tissue Sarcoma | Squamous Cell Carcinoma | Stomach (Gastric) | Throat Cancer | Thymoma |
| | | Cancer | | |
| Thymic Carcinoma | Thyroid Cancer | Transitional Cell | Carcinoma of Unknown | Ureter and Renal |
| | | Cancer of the Renal | Primary | Pelvis |
| | | Pelvis and Ureter | | |
| Transitional Cell Cancer | Urethral Cancer | Uterine Cancer | Vaginal Cancer | Vulvar Cancer |
| Wilms Tumor | | | | |

The types of cancer that can be treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some embodiments, the compositions are used to treat multiple cancer types concurrently. The compositions can also be used to treat metastases or tumors at multiple locations.

2. Immune System Disorders

Immune system disorders can also be treated. Non-limiting examples of immune system disorders include 22q11.2 deletion syndrome, Achondroplasia and severe combined immunodeficiency, Adenosine Deaminase 2 deficiency, Adenosine deaminase deficiency, Adult-onset immunodeficiency with anti-interferon-gamma autoantibodies, Agammaglobulinemia, non-Bruton type, Aicardi-Goutieres syndrome, Aicardi-Goutieres syndrome type 5, Allergic bronchopulmonary aspergillosis, Alopecia, Alopecia totalis, Alopecia universalis, Amyloidosis AA, Amyloidosis familial visceral, Ataxia telangiectasia, Autoimmune lymphoproliferative syndrome, Autoimmune lymphoproliferative syndrome due to CTLA4 haploinsuffiency, Autoimmune polyglandular syndrome type 1, Autosomal dominant hyper IgE syndrome, Autosomal recessive early-onset inflammatory bowel disease, Autosomal recessive hyper IgE syndrome, Bare lymphocyte syndrome 2, Barth syndrome, Blau syndrome, Bloom syndrome, Bronchiolitis obliterans, C1q deficiency, Candidiasis familial chronic mucocutaneous, autosomal recessive, Cartilage-hair hypoplasia, CHARGE syndrome, Chediak-Higashi syndrome, Cherubism, Chronic atypical neutrophilic dermatosis with lipodystrophy and elevated temperature, Chronic graft versus host disease, Chronic granulomatous disease, Chronic Infantile Neurological Cutaneous Articular syndrome, Chronic mucocutaneous candidiasis (CMC), Cohen syndrome, Combined immunodeficiency with skin granulomas, Common variable immunodeficiency, Complement component 2 deficiency, Complement component 8 deficiency type 1, Complement component 8 deficiency type 2, Congenital pulmonary alveolar proteinosis, Cryoglobulinemia, Cutaneous mastocytoma, Cyclic neutropenia, Deficiency of interleukin-1 receptor antagonist, Dendritic cell, monocyte, B lymphocyte, and natural killer lymphocyte deficiency, Dyskeratosis congenital, Dyskeratosis congenita autosomal dominant, Dyskeratosis congenita autosomal recessive, Dyskeratosis congenita X-linked, Epidermodysplasia verruciformis, Familial amyloidosis, Finnish type, Familial cold autoinflammatory syndrome, Familial Mediterranean fever, Familial mixed cryoglobulinemia, Felty's syndrome, Glycogen storage disease type 1B, Griscelli syndrome type 2, Hashimoto encephalopathy, Hashimoto's syndrome, Hemophagocytic lymphohistiocytosis, Hennekam syndrome, Hepatic venoocclusive disease with immunodeficiency, Hereditary folate malabsorption, Hermansky Pudlak syndrome 2, Herpes simplex encephalitis, Hoyeraal Hreidarsson syndrome, Hyper IgE syndrome, Hyper-IgD syndrome, ICF syndrome, Idiopathic acute eosinophilic pneumonia, Idiopathic CD4 positive T-lymphocytopenia, IL12RB1 deficiency, Immune defect due to absence of thymus, Immune dysfunction with T-cell inactivation due to calcium entry defect 1, Immune dysfunction with T-cell inactivation due to calcium entry defect 2, Immunodeficiency with hyper IgM type 1, Immunodeficiency with hyper IgM type 2, Immunodeficiency with hyper IgM type 3, Immunodeficiency with hyper IgM type 4, Immunodeficiency with hyper IgM type 5, Immunodeficiency with thymoma, Immunodeficiency without anhidrotic ectodermal dysplasia, Immunodysregulation, polyendocrinopathy and enteropathy X-linked, Immunoglobulin A deficiency 2, Intestinal atresia multiple, IRAK-4 deficiency, Isolated growth hormone deficiency type 3, Kawasaki disease, Large granular lymphocyte leukemia, Leukocyte adhesion deficiency type 1, LRBA deficiency, Lupus, Lymphocytic hypophysitis, Majeed syndrome, Melkersson-Rosenthal syndrome, MHC class 1 deficiency, Muckle-Wells syndrome, Multifocal fibrosclerosis, Multiple sclerosis, MYD88 deficiency, Neonatal systemic lupus erythematosus, Netherton syndrome, Neutrophil-specific granule deficiency, Nijmegen breakage syndrome, Omenn syndrome, Osteopetrosis autosomal recessive 7, Palindromic rheumatism, Papillon Lefevre syndrome, Partial androgen insensitivity syndrome, PASLI disease, Pearson syndrome, Pediatric multiple sclerosis, Periodic fever, aphthous stomatitis, pharyngitis and adenitis, PGM3-CDG, Poikiloderma with neutropenia, Pruritic urticarial papules plaques of pregnancy, Purine nucleoside phosphorylase deficiency, Pyogenic arthritis, pyoderma gangrenosum and acne, Relapsing polychondritis, Reticular dysgenesis, Sarcoidosis, Say Barber Miller syndrome, Schimke immunoosseous dysplasia, Schnitzler syndrome, Selective IgA deficiency, Selective IgM deficiency, Severe combined immunodeficiency, Severe combined immunodeficiency due to complete RAG1/2 deficiency, Severe combined immunodeficiency with sensitivity to ionizing radiation, Severe combined immunodeficiency, Severe congenital neutropenia autosomal recessive 3, Severe congenital neutropenia X-linked, Shwachman-Diamond syndrome, Singleton-Merten syndrome, SLC35C1-CDG (CDG-IIc), Specific antibody deficiency, Spondyloenchondrodysplasia, Stevens-Johnson syndrome, T-cell immunodeficiency, congenital alopecia and nail dystrophy, TARP syndrome, Trichohepatoenteric syndrome, Tumor necrosis factor receptor-associated periodic syndrome, Twin to twin transfusion syndrome, Vici syndrome, WHIM syndrome, Wiskott Aldrich syndrome, Woods Black Norbury syndrome, X-linked agammaglobulinemia, X-linked lymphoproliferative syndrome, X-linked lymphoproliferative syndrome 1, X-linked lymphoproliferative syndrome 2, X-linked magnesium deficiency with Epstein-Barr virus infection and neoplasia, X-linked severe combined immunodeficiency, and ZAP-70 deficiency.

The compositions and methods can also be used to treat autoimmune diseases or disorders. Exemplary autoimmune diseases or disorders, which are not mutually exclusive with the immune system disorders described above, include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

Effective Amounts

The effective amount or therapeutically effective amount of a pharmaceutical composition can be a dosage sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder such as cancer.

In some embodiments, administration of the pharmaceutical compositions elicits an anti-cancer response, the amount administered can be expressed as the amount effective to achieve a desired anti-cancer effect in the recipient. For example, in some embodiments, the amount of the pharmaceutical compositions is effective to inhibit the viability or proliferation of cancer cells in the recipient. In some embodiments, the amount of pharmaceutical compositions is effective to reduce the tumor burden in the recipient, or reduce the total number of cancer cells, and combinations thereof. In other embodiments, the amount of the pharmaceutical compositions is effective to reduce one or more symptoms or signs of cancer in a cancer patient. Signs of cancer can include cancer markers, such as PSMA levels in the blood of a patient.

The effective amount of the pharmaceutical compositions required will vary from subject to subject, depending on the species, age, weight and general condition of the subject, the severity of the disorder being treated, characteristics of the therapeutic compound (including activity, pharmacokinetics, pharmacodynamics, and bioavailability), and its mode of administration. Thus, it is not possible to specify an exact amount for every pharmaceutical compositions. However, an appropriate amount can be determined by one of ordinary skill in the art using only routine experimentation given the disclosed teachings. For example, effective dosages and schedules for administering the pharmaceutical compositions can be determined empirically, and making such determinations is within the skill in the art. For additional guidance, see Remington: The Science and Practice of Pharmacy (Gennaro ed. 20th edition, Williams & Wilkins PA, USA) (2000). In some embodiments, the dosage ranges for the administration of the compositions are those large enough to effect reduction in cancer cell proliferation or viability, or to reduce tumor burden for example.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition used for treatment can increase or decrease over the course of a particular treatment. Changes in dosage can result and become apparent from the results of diagnostic assays.

In general, a pharmaceutical composition including the CAR T cells can be administered at a dosage of 10⁴ to 10⁹ cells/kg body weight, preferably 10⁵ to 10⁶ cells/kg body weight, including all integer values within those ranges. CAR T cell compositions can also be administered once or multiple times at these dosages. The cells can be administered by using infusion techniques that are commonly known in immunotherapy. The optimal dosage and treatment regime for a particular patient can readily be determined by one skilled in the art of medicine by monitoring the patient for signs of disease and adjusting the treatment accordingly. In some embodiments, the unit dosage is in a unit dosage form for intravenous injection, oral administration, inhalation, or intratumoral injection.

Treatment can be continued for an amount of time sufficient to achieve one or more desired therapeutic goals, for example, a reduction of the amount of cancer cells relative to the start of treatment, or complete absence of cancer cells in the recipient. The progression of treatment can be monitored using any means known for monitoring the progression of anti-cancer treatment in a patient. In some embodiments, administration is carried out every day of treatment, or every week, or every fraction of a week. In some embodiments, treatment regimens are carried out over the course of up to two, three, four or five days, weeks, or months, or for up to 6 months, or for more than 6 months, for example, up to one year, two years, three years, or up to five years.

Combination Therapies

The compositions can be administered alone or in combination with one or more conventional therapies, for example, a conventional therapy for the disease or disorder being treated. In some embodiments, the conventional therapy includes administration of one or more of the compositions in combination with one or more additional active agents. The additional active agent(s) can have the same, or different mechanisms of action. In some embodiments, the combination results in an additive effect on the treatment of the disease or disorder (e.g., cancer). In some embodiments, the combinations result in a more than additive effect on the treatment of the disease or disorder.

The additional therapy or procedure can be simultaneous or sequential with the administration of the composition. In some embodiments the additional therapy is performed between drug cycles or during a drug holiday that is part of the composition's dosage regime.

Combination therapy may be achieved by use of a single pharmaceutical composition that includes the therapeutic agents, or by administering two or more distinct compositions at the same or different time. The multiple therapies may be given in either order and may precede or follow the other treatment by intervals ranging from minutes to weeks. In embodiments where the other agents are applied separately, it is preferable to administer the therapies in time frames, such that the agents would still be able to exert an advantageously combined effect on the patient. In such instances, it is contemplated that one may administer both modalities within about 12-24 h of each other and, more preferably, within about 6-12 h of each other. In some situations, it may be desirable to extend the time period for treatment, however, where several days (e.g., 2, 3, 4, 5, 6, 7, 8, 9, 10, or more) to several weeks (e.g., 1, 2, 3, 4, 5, 6, 7, 8 or more) lapse between the respective administrations.

In some embodiments, the additional therapy or procedure is surgery, radiotherapy, chemotherapy, immunotherapy, cancer vaccines (e.g. dendritic cell vaccine), cryotherapy or gene therapy. Immunotherapy includes, but is not limited to, administration of one or more immune-checkpoint blockage agents. Exemplary immune-checkpoint blockage agents include, but are not limited to, an antibody or antigen-binding fragment thereof, such as an antibody or antigen-binding fragment thereof that is an inhibitor of CTLA-4, PD-1, PD-L1, PD-L2, TIM-3, LAG3, or a combination thereof, such as Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), Ipilimumab (anti-CTLA4 mAb), and RG7876 (CD40 agonist mAb).

Additional therapeutic agents that are suitable for used in combination therapy include conventional cancer therapeutics such as chemotherapeutic agents, cytokines, and chemokines. Chemotherapeutic agents that can be used include, but are not limited to, alkylating agents, antimetabolites, antimitotics, anthracyclines, cytotoxic antibiotics, topoisomerase inhibitors, and combinations thereof. Monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors) can also be used. Other suitable anti-cancer agents include angiogenesis inhibitors including antibodies to vascular endothelial growth factor (VEGF) such as bevacizumab (AVASTIN®) and rhuFAb V2 (ranibizumab, LUCENTIS®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®).

Additional representative chemotherapeutic agents that can be used include, but are not limited to, amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, taxol and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In some embodiments, the compositions and methods are used prior to or in conjunction with surgical removal of tumors, for example, in preventing primary tumor metastasis. In some embodiments, the compositions and methods are used to enhance the body's own anti-tumor immune functions.

### V. Kits

The gene editing compositions, reagents, compositions and other materials can be packaged together in any suitable combination as a kit useful for performing, or aiding in the performance of, the methods. It is useful if the components in a given kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

Provided are kits containing an RNA-guided endonuclease (e.g., Cpf1), an AAV crRNA library, and instructional material for use thereof. In preferred embodiments, the library includes a plurality of vectors, where each vector independently contains a crRNA expression cassette encoding one or more crRNAs (e.g., 2 distinct crRNAs) and optionally, a CAR expression cassette. In some embodiments, the kit can contain a population of cells (e.g., T cells) collectively containing the AAV crRNA library. The instructional material can include a publication, a recording, a diagram, or any other medium of expression which can be used to communicate the usefulness of the compositions and methods of the kit. For example, the instructional material may provide instructions for methods using the kit components, such as performing transfections, transductions, infections, and conducting screens.

It is to be understood that the methods and compositions are not limited to specific synthetic methods, specific analytical techniques, or to particular reagents unless otherwise specified, and, as such, can vary. It is also to be understood that the terminology used is for the purpose of describing particular embodiments only and is not intended to be limiting.

The present invention can be further understood by reference to the following number paragraphs.

1. A library comprising a plurality of two or more vectors, each vector comprising:
one or more inverted terminal repeat (ITR) sequences, a 5′ homology arm, a crRNA expression cassette, a chimeric antigen receptor (CAR) expression cassette, and a 3′ homology arm.

2. The library of paragraph 1, wherein the crRNA expression cassette of each vector independently encodes a first guide RNA and a second guide RNA, wherein the first guide RNA is identical across the plurality of vectors.

3. The library of paragraphs 1 or 2, wherein the second guide RNA is unique to each vector across the plurality of vectors.

4. The library of any one of paragraphs 1-3, wherein one or more sequences encoding the one or more of the encoded guide RNAs of the library are selected from the group consisting of SEQ ID NOs:3-12,134.

5. The library of any one of paragraphs 1-4, wherein the library collectively comprises from about 100 to about 300,000, from about 1,000 to about 5,000 or from about 5000, to about 10,000 distinct guide RNAs.

6. The library of any one of paragraphs 1-7, wherein the library collectively comprises guide RNAs encoded by SEQ ID NOs:3-4,087 (Rene library), SEQ ID NOs:4,088-12,134 (Descartes library), or SEQ ID NOs:3-12,134.

7. The library of any one of paragraphs 1-6, wherein each crRNA expression cassette comprises a U6 promoter operably linked to sequences encoding one or more guide RNAs.

8. The library of any one of paragraphs 1-7, wherein each crRNA expression cassette comprises sequences encoding a first guide RNA and a second guide RNA.

9. The library of any one of paragraphs 1-8, wherein the CAR expression cassette comprises an EFS promoter and/or a polyadenylation signal sequence operationally linked to a sequence encoding the CAR.

10. The library of any one of paragraphs 1-9, wherein the crRNA expression cassette and/or CAR expression cassette of each vector are positioned between the 5′ and 3′ homology arms.

11. The library of any one of paragraphs 1-10, wherein the 5′ and 3′ homology arms are homologous to the TRAC locus.

12. The library of any one of paragraphs 1-11, wherein each vector encodes at least one guide RNA targeting the TRAC locus.

13. The library of any one of paragraphs 1-12, wherein the CAR targets one or more cancer specific antigens or cancer associated antigens.

14. The library of any one of paragraphs 1-13, wherein the CAR is an anti-CD19 CAR or anti-CD22 CAR.

15. The library of any one of paragraphs 2-14, wherein the second guide RNA targets a gene involved in T cell exhaustion, T cell proliferation, T cell co-stimulation, memory T cell differentiation, T cell receptor signaling, epigenetic regulation, adaptive immune response, immune response to tumor cells, other immune functions, or combinations thereof.

16. The library of any one of paragraphs 1-15, wherein the vector comprises the nucleotide sequence of SEQ ID NO:1 or SEQ ID NO:2 with or without the sequence encoding the TRAC targeting crRNA, with or without one or more additional crRNA encoding sequences optionally inserted at the BbsI cloning site, and/or with the existing CAR encoding sequence or another CAR encoding sequence substituted therefore, or a sequence variant having 75% or more sequence identity to any of the foregoing.

17. The library of any one of paragraphs 1-16, wherein each vector is a viral vector, preferably an adeno-associated virus (AAV) vector, optionally wherein the AAV is AAV6.

18. A vector of any one of paragraphs 1-17.

19. A population of cells comprising an AAV vector of paragraph 18.

20. A population of cells collectively comprising the library of any one of paragraphs 1-17, optionally wherein each cell comprises at most one or two AAV vectors comprised in the library.

21. A method of identifying one or more genes that enhance a desired phenotype of a cell comprising a CAR, the method comprising:
(a) contacting the population of cells of paragraph 20 with an RNA-guided endonuclease under conditions suitable for genomic integration and expression of the guide RNAs and CAR contained in the vectors; and
(b) selecting for cells exhibiting the desired phenotype.

22. The method of paragraph 21, wherein the crRNA expression cassette and CAR expression cassette are integrated into the TRAC locus.

23. The method of paragraph 21 or 22, wherein the RNA-guided endonuclease is provided as an mRNA that encodes the RNA-guided endonuclease, a viral vector that encodes the RNA-guided endonuclease, or an RNA-guided endonuclease protein or a complex of the RNA-guided endonuclease protein and RNA.

24. The method of paragraph 23, wherein the RNA-guided endonuclease is provided by electroporation.

25. The method of any one of paragraphs 21-24, wherein the RNA-guided endonuclease is Cpf1 or an active variant, derivative, or fragment thereof.

26. The method of any one of paragraphs 21-25, wherein the desired phenotype is selected from the group comprising increased tumor/tumor microenvironment infiltration, increased or optimized target cell affinity, increased target cell cytotoxicity, increased persistence, increased expansion/proliferation, reduced exhaustion, increased anti-cancer metabolic function, increased ability to prevent immune escape, reduced unspecific cytokine production, reduced off-target toxicity, reduced cytokine release syndrome (CRS), and combinations thereof.

27. The method of any one of paragraphs 21-26, wherein the step of selecting comprises co-culturing the population of cells with target cells comprising one or more antigens recognized by the CAR for a defined time period, flow cytometry-based or affinity-based sorting, immune marker-based selection, in vivo tumor infiltration, CAR-antigen interaction, directed evolution, or combinations thereof.

28. The method of paragraph 27, wherein the population of cells is repeatedly co-cultured with the target cells.

29. The method of paragraph 27 or 28, wherein the time period comprises from about 1 to about 60 days.

30. The method of any one of paragraphs 27-29, wherein the target cells comprise cancer cells.

31. The method of any one of paragraphs 21-30, further comprising identifying the crRNA expression cassette present in the selected cells.

32. The method of paragraph 31, wherein the step of identifying the crRNA expression cassette comprises sequencing genomic DNA of the selected cells.

33. The method of paragraph 31 or 32, wherein the one or more genes that enhance a desired phenotype are identified as genes targeted by the guide RNAs encoded by the crRNA expression cassette.

34. The method of any one of paragraphs 21-33, wherein the population of cells comprises effector T cells, memory T cells, central memory T cells, effector memory T cells, Th1 cells, Th2 cells, Th3 cells, Th9 cells, Th17 cells, Tfh cells, Treg cells, gamma-delta T cells, hematopoietic stem cells (HSC), macrophages, natural killer cells (NK), B cells, dendritic cells (DC), or other immune cells.

35. The method of paragraph 34, wherein the T cells are CD4⁺ or CD8⁺ T cells.

36. An isolated CAR T cell comprising a CAR and one or more mutations in one or more genes identified by the method of any one of paragraphs 21-35.

37. The CAR T cell of paragraph 36, wherein the one or more mutations cause reduced function of the one or more genes or gene products thereof.

38. The CAR T cell of paragraph 36 or 37, wherein the one or more genes is selected from the group comprising PRDM1, DPF3, SLAMF1, TET2, HFE, PELI1, PDCD1, HAVCR2IM3, TET2, NR4A2, LAIR1, and USB1.

39. The CAR T cell of any one of paragraphs 36-38, wherein the cell exhibits increased memory, increased cell proliferation, increased persistence, increased cytotoxicity towards a target cell, decreased T cell terminal differentiation, and/or reduced T cell exhaustion compared to a CAR T cell not comprising the one or more mutations in the one or more genes.

40. A population of CAR T cells derived by expanding the CAR T cell of any one of paragraphs 36-39.

41. A pharmaceutical composition comprising the population of CAR T cells of paragraph 40 and a pharmaceutically acceptable buffer, carrier, diluent or excipient.

42. A method of treating a subject having a disease, disorder, or condition comprising administering to the subject an effective amount of the pharmaceutical composition of paragraph 41.

43. The method of paragraph 42, wherein the disease, disorder, or condition is associated with an elevated expression or specific expression of an antigen.

44. The method of paragraph 43, wherein the CAR T cell targets the antigen.

45. The method of any one of paragraphs 42-44, wherein the cell was isolated from a healthy donor or from the subject having the disease, disorder, or condition prior to the introduction of the one or more mutations in the one or more genes.

46. The method of any one of paragraphs 42-45, wherein the disease, disorder, or condition is a cancer, an inflammatory disease, a neuronal disorder, HIV/AIDS, diabetes, a cardiovascular disease, an infectious disease, or an autoimmune disease.

47. The method of paragraph 46, wherein the cancer is a leukemia or lymphoma selected from the group comprising chronic lymphocytic leukemia (CLL), acute lymphocytic leukemia (ALL), acute myeloid leukemia (AML), chronic myelogenous leukemia (CML), mantle cell lymphoma, non-Hodgkin's lymphoma, and Hodgkin's lymphoma.

48. The method of any one of paragraphs 42-47, wherein the subject is a human.

49. A cell comprising a heterologous nucleic acid construct comprising one or more crRNA expression cassettes comprising a nucleic acid sequence encoding one or more guide RNAs selected from the group consisting of SEQ ID NOs:3-12,134 and a chimeric antigen receptor (CAR) expression cassette.

50. A cell comprising a heterologous nucleic acid construct encoding a chimeric antigen receptor (CAR) expression cassette and reduced or eliminated expression at one or more gene loci targeted by one or more guide RNAs selected from the group consisting of SEQ ID NOs:3-12,134 and.

51. The cell of paragraphs 49 and 50, wherein the heterologous nucleic acid construct is present in the cell's genome at the TRAC gene locus, and optionally wherein the CAR is an anti-CD19 or anti-CD22 CAR.

The present invention will be further understood by reference to the following non-limiting examples.

### EXAMPLES

### Example 1: Establishment of the CLASH System for Massively Parallel CAR-T Engineering

### Materials and Methods

Design and Generation of CLASH AAV Construct

To generate the CLASH AAV vector (pAAV-LHA-U6-DR-cfTRAC-DR-BbsI-EFS-CAR-scFv-RHA, or pXD60), the TRAC HDR arms and CD22BBz/CD19BBz CAR were amplified as previously reported (Dai X., et al., Nat. Methods, 16:247-254 (2019)). The crRNA cassette which contains one guide targeting the first exon of the TRAC locus and double BbsI cutting sites, was inserted after the left TRAC arm. Different fragments were cloned using Gibson assembly and traditional restriction cloning.

Design of Rene and Descartes Cas12a/Cpf1 crRNA Libraries

The Descartes library contains 8,047 crRNAs (SEQ ID NOs:4,088-12,134), targeting 954 genes (see Table 2) with ˜8 crRNAs per gene and 1000 non-targeting controls (NTCs). Genes of interest were chosen as a superset from the following gene lists: T cell exhaustion (Wherry E J., et al., Immunity 27, 670-684 (2007)), epigenetic regulators (Arrowsmith C H., et al., Nature reviews Drug discovery 11, 384-400 (2012)), T cell co-stimulation (GO: 0031295), memory T cell differentiation (GO: 0043379), T cell receptor signaling pathway (GO: 0050852), adaptive immune response (GO: 0002250), immune response to tumor cell (GO: 0002418), T cell proliferation (GO: 0042098), and TE72. The Rene library contains 4,085 crRNAs (SEQ ID NOs:3-4,087) targeting 472 genes (see Table 3) including T cell exhaustion, epigenetic regulators, T cell co-stimulation (GO: 0031295), memory T cell differentiation (GO: 0043379), and TE72. A total of 500 non-targeting control (NTC) crRNAs were spiked into the Rene library. All crRNAs were scored for selection using Deep Cpf1 (Kim H K., et al., Nat Biotechnol., 36(3):239-241 (2018)).

TABLE 2
Genes targeted by crRNAs in the Descartes library.

| | | | | | | | |
|---|---|---|---|---|---|---|---|
| ABL1 | ACTN1 | ADA | ADAM15 | ADAM17 | ADGRE1 | ADGRG1 | ADRB3 |
| AEBP2 | AGER | AGFG1 | AHR | AICDA | AIF1 | AIRE | AKAP1 |
| AKT1 | ALCAM | ALOX15 | ANXA1 | APCS | APLF | ARG1 | ARG2 |
| ARHGEF15 | ARID4A | ARID4B | ART3 | ASH1L | ASXL1 | ASXL3 | ATAD2 |
| ATAD2B | ATAD5 | ATAT1 | ATF1 | B2M | BATF | BAX | BAZ1A |
| BAZ1B | BAZ2A | BAZ2B | BCAN | BCAR1 | BCL10 | BCL2 | BCL3 |
| BCL6 | BCLAF3 | BLM | BMP4 | BMX | BPTF | BRD1 | BRD2 |
| BRD3 | BRD4 | BRD7 | BRD8 | BRD9 | BRDT | BRPF1 | BRPF3 |
| BRWD1 | BRWD3 | BTK | BTLA | BTN1A1 | BTN2A1 | BTN2A2 | BTN3A1 |
| BTN3A2 | BTN3A3 | BTNL2 | BTNL3 | BTNL8 | BTNL9 | BUB1 | C17orf99 |
| C1QA | C1QB | C1QBP | C1QC | C1R | C1RL | C1S | C2 |
| C20orf196 | C3 | C4A | C4B | C4BPA | C4BPB | C5 | C6 |
| C7 | C8A | C8B | C8G | C9 | CA2 | CACNA1F | CADM1 |
| CALU | CAMK4 | CARD11 | CARM1 | CASP3 | CAV1 | CBX1 | CBX2 |
| CBX3 | CBX4 | CBX5 | CBX6 | CBX7 | CBX8 | CCDC88B | CCL19 |
| CCL21 | CCL3 | CCL4 | CCL5 | CCND3 | CCR2 | CCR6 | CCR7 |
| CCRL2 | CD151 | CD160 | CD1A | CD1B | CD1C | CD1D | CD1E |
| CD200 | CD209 | CD226 | CD24 | CD244 | CD247 | CD27 | CD274 |
| CD276 | CD28 | CD300A | CD3D | CD3E | CD3G | CD4 | CD40 |
| CD40LG | CD46 | CD48 | CD5 | CD55 | CD6 | CD7 | CD74 |
| CD79A | CD79B | CD80 | CD84 | CD86 | CD8A | CD8B | CD9 |
| CDC42 | CDH4 | CDY1 | CDY2A | CDYL | CDYL2 | CEACAM1 | CEBPB |
| CECR2 | CFI | CHD1 | CHD2 | CHD3 | CHD4 | CHD5 | CHD6 |
| CHD7 | CHD8 | CHD9 | CHUK | CKS2 | CLC | CLCF1 | CLEC10A |
| CLEC4A | CLEC4C | CLEC4D | CLEC4G | CLEC4M | CLEC6A | CLECL1 | CLIP1 |
| CLIP4 | CLOCK | CLU | COCH | COL7A1 | CORO1A | CR1 | CR2 |
| CRACR2A | CREBBP | CRLF1 | CRP | CRTAM | CSK | CTLA4 | CTNNB1 |
| CTPS1 | CTSC | CTSH | CTSL | CTSS | CXCL13 | CXCR4 | CXXC1 |
| CYLD | CYP4V2 | DBNL | DCLRE1C | DDIT4 | DENND1B | DENND5A | DHPS |
| DIDO1 | DLG1 | DLG5 | DMRTC2 | DNAH12 | DNAJA3 | DND1 | DNMT3A |
| DNMT3B | DOCK2 | DOCK3 | DOCK8 | DOT1L | DPF1 | DPF2 | DPF3 |
| DPP4 | DPPA3 | DQX1 | DUSP1 | DUSP10 | DUSP22 | DUSP3 | DYNLT3 |
| EBI3 | EFNB1 | EFNB3 | EGR2 | EHMT1 | EHMT2 | EIF2AK4 | EIF2B1 |
| EIF2B2 | EIF2B3 | EIF2B4 | EIF2B5 | ELF1 | ELF4 | ELP3 | EMP2 |
| ENTPD1 | EOMES | EP300 | EPO | ERAP1 | ERAP2 | ERAS | ERBB2 |
| ERCC1 | ERMAP | EXO1 | EXOSC3 | EXOSC6 | EZH1 | EZH2 | EZR |
| FABP3 | FADD | FAM35A | FAM49B | FASLG | FCAMR | FCER1G | FCER2 |
| FCGR1B | FCGR2B | FCRL4 | FGA | FGB | FKBP14 | FKBP1B | FKBP7 |
| FMR1 | FOXJ1 | FOXP3 | FSD1 | FXR2 | FYB1 | FYB2 | FYN |
| FZD5 | G2E3 | GABBR1 | GAPT | GAS2 | GATA3 | GBP1 | GCNT3 |
| GLMN | GLYR1 | GNL1 | GNRH1 | GPAM | GPD2 | GPNMB | GPR162 |
| GPR180 | GPR183 | GPR65 | GRAP2 | GRB2 | GRIK5 | GTF3C4 | HAT1 |
| HAVCR2 | HDAC1 | HDAC10 | HDAC11 | HDAC2 | HDAC3 | HDAC4 | HDAC5 |
| HDAC6 | HDAC7 | HDAC8 | HDAC9 | HDGF | HDGFL1 | HDGFL2 | HDGFL3 |
| HES1 | HFE | HHLA2 | HLA-A | HLA-B | HLA-C | HLA-DMB | HLA-DPA1 |
| HLA-DPB1 | HLA-DQA1 | HLA-DQA2 | HLA-DQB1 | HLA-DQB2 | HLA-DRA | HLA-DRB1 | HLA-DRB5 |
| HLA-E | HLA-F | HLA-G | HLX | HMGB1 | HMHB1 | HORMAD1 | HPX |
| HRAS | HRG | HSPD1 | ICAM1 | ICOS | ICOSLG | IDO1 | IER5 |
| IFNA1 | IFNA10 | IFNA14 | IFNA16 | IFNA17 | IFNA2 | IFNA21 | IFNA4 |
| IFNA5 | IFNA6 | IFNA7 | IFNA8 | IFNB1 | IFNE | IFNG | IFNK |
| IFNL1 | IFNW1 | IGF1 | IGF2 | IGFBP2 | IGLL1 | IGLL5 | IGLON5 |
| IHH | IKBKB | IKBKG | IL10 | IL12A | IL12B | IL12RB1 | IL13RA2 |
| IL15 | IL18 | IL18BP | IL18R1 | IL18RAP | IL1B | IL1R1 | IL1RL1 |
| IL2 | IL20RB | IL21 | IL23A | IL23R | IL27 | IL27RA | IL2RA |
| IL31RA | IL33 | IL4 | IL4R | IL6 | IL6R | IL6ST | IL7R |
| ING1 | ING2 | ING3 | ING4 | ING5 | INPP5D | INTS12 | IRF1 |
| IRF4 | IRF7 | IRF8 | ISG15 | ISG20 | ITCH | ITGAV | ITK |
| ITM2A | ITPR2 | JADE1 | JADE2 | JADE3 | JAG1 | JAK2 | JAK3 |
| JAM3 | JARID2 | JCHAIN | JMJD1C | KAT2A | KAT2B | KAT5 | KAT6A |
| KAT6B | KAT7 | KAT8 | KCNN4 | KDELR1 | KDM1A | KDM1B | KDM2A |
| KDM2B | KDM3A | KDM3B | KDM4A | KDM4B | KDM4C | KDM4D | KDM4E |
| KDM5A | KDM5B | KDM5C | KDM5D | KDM6A | KDM6B | KDM7A | KDM8 |
| KLHL6 | KLK1 | KLK6 | KLRK1 | KMT2A | KMT2B | KMT2C | KMT2D |
| KMT2E | KMT5A | KMT5B | KMT5C | L3MBTL1 | L3MBTL2 | L3MBTL3 | L3MBTL4 |
| LAG3 | LAIR1 | LAMP3 | LAT | LAT2 | LAX1 | LBR | LCK |
| LCP2 | LEF1 | LEP | LGALS1 | LGALS3 | LGALS9 | LIG4 | LILRA1 |
| LILRA6 | LILRB1 | LILRB2 | LILRB3 | LILRB4 | LILRB5 | LIME1 | LMO1 |
| LOXL3 | LRRC32 | LTA | LY75 | LY9 | LYN | MAD1L1 | MAD2L2 |
| MALT1 | MAN2B1 | MANSC1 | MAP3K1 | MAP3K7 | MAP3K8 | MAP7D2 | MAPK1 |
| MARCH7 | MARCH8 | MASP2 | MBD5 | MBL2 | MBTD1 | MCOLN1 | MCOLN2 |
| MDFIC | MECOM | MEF2C | MEF2D | MICA | MICB | MLH1 | MLLT10 |
| MLLT6 | MOG | MORF4L1 | MPHOSPH8 | MSH2 | MSH6 | MSL3 | MSN |
| MTF2 | MTMR7 | MUM1 | MYH10 | MYO1G | NAMPT | NBN | NCK1 |
| NCK2 | NCKAP1L | NCOA1 | NCOA3 | NCSTN | NDFIP1 | NDUFA5 | NECTIN2 |
| NFATC1 | NFKB1 | NFKB2 | NFKBID | NFKBIZ | NLRP10 | NLRP3 | NR4A2 |
| NRIP1 | NSD1 | NSD2 | NSD3 | OTUB1 | OTUD7B | P2RX4 | P2RX7 |
| PAG1 | PAK1 | PAK2 | PAK3 | PAWR | PAXIP1 | PBRM1 | PBX3 |
| PDCD1 | PDCD1LG2 | PDE4B | PDE4D | PDE5A | PDPK1 | PELI1 | PENK |
| PHF1 | PHF10 | PHF11 | PHF12 | PHF13 | PHF14 | PHF19 | PHF2 |
| PHF20 | PHF20L1 | PHF21A | PHF21B | PHF23 | PHF3 | PHF5A | PHF6 |
| PHF7 | PHF8 | PHIP | PHPT1 | PHRF1 | PIK3CA | PIK3CB | PIK3CD |
| PIK3CG | PIK3R1 | PIK3R2 | PKN1 | PLA2G2D | PLA2G2F | PLCG1 | PLCG2 |
| PLSCR1 | PNP | POU2F2 | PPM1B | PPP3CB | PRDM1 | PRDM10 | PRDM11 |
| PRDM12 | PRDM13 | PRDM14 | PRDM15 | PRDM16 | PRDM2 | PRDM4 | PRDM5 |
| PRDM6 | PRDM7 | PRDM8 | PRDM9 | PRF1 | PRKAR1A | PRKAR1B | PRKCB |
| PRKCD | PRKCQ | PRKCZ | PRKD2 | PRMT1 | PRMT2 | PRMT3 | PRMT5 |
| PRMT6 | PRMT7 | PRMT8 | PRNP | PRR36 | PRR7 | PSEN1 | PSIP1 |
| PTGER2 | PTGER4 | PTK2B | PTPN11 | PTPN13 | PTPN2 | PTPN22 | PTPN6 |
| PTPRC | PTPRJ | PVR | PVRIG | PWWP2B | PXDN | PYCARD | PYGO1 |
| PYGO2 | RAB27A | RAB29 | RAC1 | RAC2 | RAET1E | RAET1G | RAET1L |
| RAG1 | RAG2 | RAI1 | RASAL3 | RBCK1 | RBPJ | RC3H1 | RC3H2 |
| RCN1 | RELA | RELB | RFTN1 | RGS16 | RIF1 | RIOX1 | RIOX2 |
| RIPK2 | RIPK3 | RNF11 | RNF125 | RNF168 | RNF17 | RNF19B | RNF31 |
| RNF8 | RORA | RORC | RPH3A | RPS3 | RPS6 | RSAD2 | RSF1 |
| RYR1 | SAMSN1 | SASH3 | SATB1 | SCGB1A1 | SCMH1 | SCML2 | SDC4 |
| SEC61A2 | SELENOK | SEMA4A | SEPT1 | SERPINA1 | SERPING1 | SETD1A | SETD2 |
| SETD3 | SETD4 | SETD5 | SETD6 | SETD7 | SETDB1 | SETDB2 | SETMAR |
| SFMBT1 | SFMBT2 | SFTPD | SGF29 | SH2D1A | SH2D1B | SH2D2A | SH3RF1 |
| SHH | SHPRH | SIGLEC10 | SIRT1 | SIRT2 | SIRT3 | SIRT4 | SIRT5 |
| SIRT6 | SIRT7 | SIT1 | SKAP1 | SLA2 | SLAMF1 | SLAMF6 | SLAMF7 |
| SLC11A1 | SLC12A2 | SLC39A3 | SMAD1 | SMAD7 | SMARCA2 | SMARCA4 | SMARCC1 |
| SMARCC2 | SMN1 | SMNDC1 | SMO | SMYD1 | SMYD2 | SMYD3 | SMYD4 |
| SMYD5 | SND1 | SOCS3 | SOCS5 | SOS1 | SP100 | SP110 | SP140 |
| SP140L | SPN | SPP1 | SPPL3 | SPRED2 | SPRY2 | SPTB | SRC |
| SRSF7 | STAG3 | STAT3 | STAT5B | STAT6 | STK11 | STK31 | STOML2 |
| STX1B | STX7 | SUPT6H | SUSD4 | SUV39H1 | SUV39H2 | SWAP70 | SYK |
| TAB2 | TAF1 | TAF1L | TAF3 | TANK | TAP1 | TAP2 | TARM1 |
| TBX21 | TCEAL9 | TCF19 | TCF20 | TDRD1 | TDRD10 | TDRD12 | TDRD15 |
| TDRD3 | TDRD5 | TDRD6 | TDRD7 | TDRD9 | TDRKH | TEC | TESPA1 |
| TET2 | TFE3 | TFEB | TFRC | TGFB1 | TGFBR2 | THEMIS | THEMIS2 |
| THOC1 | THY1 | TLR4 | TLR8 | TMEM116 | TMEM131L | TMEM43 | TMEM47 |
| TMIGD2 | TNF | TNFAIP3 | TNFRSF11A | TNFRSF13B | TNFRSF13C | TNFRSF14 | TNFRSF17 |
| TNFRSF21 | TNFRSF4 | TNFRSF9 | TNFSF13 | TNFSF13B | TNFSF14 | TNFSF18 | TNFSF4 |
| TNFSF9 | TP53BP1 | TRAF2 | TRAF6 | TRAT1 | TRIM24 | TRIM27 | TRIM28 |
| TRIM33 | TRIM47 | TRPM4 | TSC1 | TUBB2A | TWSG1 | TXK | UBASH3A |
| UBE2N | UBR7 | UHRF1 | UHRF2 | ULBP1 | ULBP2 | ULBP3 | UNC13D |
| UNC93B1 | UNG | USB1 | USP49 | UTY | VAV1 | VCAM1 | VIPR1 |
| VTCN1 | WAS | WNT4 | XCL1 | YES1 | ZAP70 | ZBTB1 | ZBTB7B |
| ZC3H12A | ZCWPW1 | ZCWPW2 | ZFP91 | ZGPAT | ZMYND11 | ZMYND8 | ZNF683 |
| ZP3 | ZP4 | | | | | | |

TABLE 3
Genes targeted by crRNAs in the Rene library.

| | | | | | | | |
|---|---|---|---|---|---|---|---|
| ACTN1 | ADGRG1 | ADRB3 | AEBP2 | AGFG1 | AHR | AIRE | AKAP1 |
| AKT1 | ARHGEF15 | ARID4A | ARID4B | ART3 | ASH1L | ASXL1 | ASXL3 |
| ATAD2 | ATAD2B | ATAT1 | ATF1 | BAZ1A | BAZ1B | BAZ2A | BAZ2B |
| BCAN | BCL2 | BCLAF3 | BPTF | BRD1 | BRD2 | BRD3 | BRD4 |
| BRD7 | BRD8 | BRD9 | BRDT | BRPF1 | BRPF3 | BRWD1 | BRWD3 |
| BTLA | BUB1 | CA2 | CALU | CARD11 | CARM1 | CASP3 | CAV1 |
| CBX1 | CBX2 | CBX3 | CBX4 | CBX5 | CBX6 | CBX7 | CBX8 |
| CCL19 | CCL21 | CCL3 | CCL4 | CCR7 | CCRL2 | CD160 | CD200 |
| CD24 | CD244 | CD274 | CD28 | CD3E | CD40LG | CD46 | CD5 |
| CD7 | CD80 | CD86 | CD9 | CDC42 | CDH4 | CDY1 | CDY2A |
| CDYL | CDYL2 | CECR2 | CHD1 | CHD2 | CHD3 | CHD4 | CHD5 |
| CHD6 | CHD7 | CHD8 | CHD9 | CKS2 | CLIP1 | CLIP4 | CLOCK |
| COCH | COL7A1 | CREBBP | CRLF1 | CSK | CTLA4 | CXCR4 | CXXC1 |
| CYP4V2 | DDIT4 | DENND5A | DIDO1 | DMRTC2 | DNAH12 | DND1 | DNMT3A |
| DNMT3B | DOCK3 | DOT1L | DPF1 | DPF2 | DPF3 | DPP4 | DPPA3 |
| DQX1 | DUSP1 | DUSP10 | DYNLT3 | EFNB1 | EFNB3 | EGR2 | EHMT1 |
| EHMT2 | ELP3 | ENTPD1 | EOMES | EP300 | ERAS | EZH1 | EZH2 |
| FABP3 | FASLG | FKBP14 | FKBP7 | FMR1 | FSD1 | FXR2 | FYN |
| G2E3 | GABBR1 | GAS2 | GLYR1 | GPD2 | GPR162 | GPR180 | GPR65 |
| GRAP2 | GRB2 | GRIK5 | GTF3C4 | HAT1 | HDAC1 | HDAC10 | HDAC11 |
| HDAC2 | HDAC3 | HDAC4 | HDAC5 | HDAC6 | HDAC7 | HDAC8 | HDAC9 |
| HDGF | HDGFL1 | HDGFL2 | HDGFL3 | HHLA2 | HORMAD1 | ICOS | ICOSLG |
| IER5 | IFNL1 | IGLON5 | IL12B | IL12RB1 | IL23A | IL23R | IL6ST |
| ING1 | ING2 | ING3 | ING4 | ING5 | INTS12 | IRF8 | ISG15 |
| ISG20 | ITGAV | ITM2A | ITPR2 | JADE1 | JADE2 | JADE3 | JAK3 |
| JARID2 | JMJD1C | KAT2A | KAT2B | KAT5 | KAT6A | KAT6B | KAT7 |
| KAT8 | KDM1A | KDM1B | KDM2A | KDM2B | KDM3A | KDM3B | KDM4A |
| KDM4B | KDM4C | KDM4D | KDM4E | KDM5A | KDM5B | KDM5C | KDM5D |
| KDM6A | KDM6B | KDM7A | KDM8 | KLK1 | KLK6 | KLRK1 | KMT2A |
| KMT2B | KMT2C | KMT2D | KMT2E | KMT5A | KMT5B | KMT5C | L3MBTL1 |
| L3MBTL2 | L3MBTL3 | L3MBTL4 | LAG3 | LBR | LCK | LGALS1 | LILRB4 |
| LY75 | LYN | MAN2B1 | MANSC1 | MAP3K1 | MAP3K8 | MAP7D2 | MBD5 |
| MBTD1 | MDFIC | MECOM | MEF2D | MLLT10 | MLLT6 | MORF4L1 | MPHOSPH8 |
| MSH6 | MSL3 | MTF2 | MTMR7 | MUM1 | MYH10 | NAMPT | NCOA1 |
| NCOA3 | NDFIP1 | NDUFA5 | NFATC1 | NR4A2 | NRIP1 | NSD1 | NSD2 |
| NSD3 | P2RX4 | PAK1 | PAK2 | PAK3 | PBRM1 | PBX3 | PDCD1 |
| PDCD1LG2 | PDPK1 | PENK | PHF1 | PHF10 | PHF11 | PHF12 | PHF13 |
| PHF14 | PHF19 | PHF2 | PHF20 | PHF20L1 | PHF21A | PHF21B | PHF23 |
| PHF3 | PHF5A | PHF6 | PHF7 | PHF8 | PHIP | PHRF1 | PIK3CA |
| PIK3R1 | PLSCR1 | PPM1B | PRDM1 | PRDM10 | PRDM11 | PRDM12 | PRDM13 |
| PRDM14 | PRDM15 | PRDM16 | PRDM2 | PRDM4 | PRDM5 | PRDM6 | PRDM7 |
| PRDM8 | PRDM9 | PRKAR1B | PRMT1 | PRMT2 | PRMT3 | PRMT5 | PRMT6 |
| PRMT7 | PRMT8 | PRR36 | PSIP1 | PTGER2 | PTGER4 | PTPN11 | PTPN13 |
| PTPN6 | PWWP2B | PXDN | PYGO1 | PYGO2 | RAC1 | RAG2 | RAI1 |
| RBPJ | RCN1 | RGS16 | RIOX1 | RIOX2 | RNF11 | RNF17 | RPH3A |
| RSF1 | RYR1 | SCMH1 | SCML2 | SEC61A2 | SEPT1 | SERPINA1 | SETD1A |
| SETD2 | SETD3 | SETD4 | SETD5 | SETD6 | SETD7 | SETDB1 | SETDB2 |
| SETMAR | SFMBT1 | SFMBT2 | SGF29 | SH2D2A | SHPRH | SIRT1 | SIRT2 |
| SIRT3 | SIRT4 | SIRT5 | SIRT6 | SIRT7 | SLC12A2 | SLC39A3 | SMAD1 |
| SMARCA2 | SMARCA4 | SMARCC1 | SMARCC2 | SMN1 | SMNDC1 | SMO | SMYD1 |
| SMYD2 | SMYD3 | SMYD4 | SMYD5 | SND1 | SOCS3 | SP100 | SP110 |
| SP140 | SP140L | SPP1 | SPRED2 | SPRY2 | SPTB | SRC | SRSF7 |
| STAG3 | STK31 | STX1B | SUV39H1 | SUV39H2 | TAF1 | TAFIL | TAF3 |
| TANK | TCEAL9 | TCF19 | TCF20 | TDRD1 | TDRD10 | TDRD12 | TDRD15 |
| TDRD3 | TDRD5 | TDRD6 | TDRD7 | TDRD9 | TDRKH | TET2 | TMEM116 |
| TMEM43 | TMEM47 | TMIGD2 | TNFRSF13C | TNFRSF14 | TNFRSF9 | TNFSF13B | TNFSF14 |
| TNFSF4 | TP53BP1 | TRIM24 | TRIM28 | TRIM33 | TRIM47 | TSC1 | TUBB2A |
| UBR7 | UHRF1 | UHRF2 | USB1 | USP49 | UTY | VAV1 | VCAM1 |
| VIPR1 | YES1 | ZCWPW1 | ZCWPW2 | ZFP91 | ZGPAT | ZMYND11 | ZMYND8 |

Library and Individual crRNA Cloning and Preparation

The Rene and Descartes libraries were synthesized by CustomArray. Both Rene and Descartes libraries were amplified using two round PCR. The PCR product was purified by PCR purification kit (Qiagen). The crRNA libraries of Descartes were cloned into the CLASH AAV plasmid by linearization with BbsI digestion and Gibson assembly. The Gibson assembly Descartes library products were transformed into high-efficiency competent cells (Endura) by electroporation. An estimated crRNA library coverage of ≥100× was observed after electroporation by colony counting. All the bacteria were harvested in pool and the plasmid library was purified using EndoFree Plasmid Maxi Kit (Qiagen). The representation of crRNAs in library plasmid was verified by NGS.

Packaging and Purification of AAV6

The Descartes library, empty vector, or individual gene targeting CLASH vectors were packaged by AAV6 serotype vectors to target human T cells. Briefly, AAV6 serotype plasmid, packing plasmid pDF6 and AAV6 transgene vector plasmid were added at a ratio of 1.7:2:1, and then polyethyleneimine was added and mixed well by vortex. The solution was left at room temperature for 10-20 minutes, then it was added dropwise onto HEK293FT cells at 80-90% confluency in 15-cm tissue culture dishes (Corning). Transfected cells were collected with PBS at 72 hours post-transfection. For AAV purification, transfected cells were mixed with pure chloroform (1:10 volume) and incubated at 37° C. with vigorous shaking for 1 hour. Pure NaCl was added to a final concentration of 1 M, then samples were centrifuged at 20,000 g at 4° C. for 15 minutes. The aqueous layer was transferred to another tube while the chloroform layer was discarded. PEG8000 was added to 10% (w/v), followed by vigorous shaking to dissolve, and the mixture was incubated at 4° C. for 1 hour. Samples were centrifuged at 20,000 g at 4° C. for 15 minutes. The supernatant was then discarded and the pellet resuspended with Dulbecco's phosphate-buffered saline (DPBS) with MgCl₂. The dissolved solution was treated with universal nuclease (Thermo Fisher), then incubated at 37° C. for 30 minutes. Chloroform was added (1:1 volume), followed by vortexing and centrifugation at 14,000 g at 4° C. for 15 minutes. The aqueous layer was dumped into a 100-kDa molecular cut-off filter (Millipore) and centrifuged at 3000 g to concentrate the virus. Virus was tittered by quantitative PCR using custom Taqman assays targeted to the U6 promoter.

Library Scale AAV Transduction

Human primary peripheral blood CD8⁺ T cells or human peripheral blood mononuclear cells (PBMC) were purchased from STEMCELL Technologies. The CD8⁺ T cells from PBMC were isolated using the human CD8+ T Cell isolation kit (Miltenyi Biotec) according to the manufacturer's protocol. T cells were cultured in X-VIVO media (Lonza) with 5% human AB serum and recombinant human IL-2 20 ng/mL. Electroporation was performed after T cells thawed for 2 days. Cells were prepared at a density of ˜2.5×10⁶ cells per 100 μL tip reaction in electroporation Buffer R (Neon Transfection System Kits). A total of 20 reactions were set for the Descartes library electroporation. For each reaction, T cells were mixed with 10 μg of modified NLS-LbCpf1mRNA (TriLink) and electric shocked at program 24 (1,600V, 10 ms and three pulses). After electroporation, the cells were transferred into 1 mL of pre-warmed X-VIVO media (with 5% human AB serum but without antibiotics) immediately. Indicated volumes of AAV6 at an estimated number of viral particles (vps) per cell (MOI=1×10³-10⁴ vp/c) were added into the T cells after electroporation for 2-4 hours. Due to the fact that there are empty/defective AAVs during packaging which make them non-infectious, the actual infectious vps are often lower, making functional infectivity lower than 1×10³-10⁴ vp/c. While it is possible that a T cell can have more than one AAV, a T cell only has two copies of genome, and therefore, the CLASH HDR knock-in design caps the crRNA integration so that each cell can have no more than 2 different integrated crRNAs. It was observed that 5 days post transduction the percentage of CAR-positive cells was 10.9%, yielding an effective multiplicity of infection (effective MOI) of 0.1096 in comparable screen terms.

Validation of Massively Parallel HDR Knock-In in Human T Cells

After electroporation for 5 days, the genomic DNA of massively parallel engineered T cell pools was extracted by using QIAamp DNA Blood Mini Kit (Qiagen). In-Out PCR was used to amplify out the integrated fragments from gDNA. The PCR product was purified by PCR purification kit (Qiagen) and sequenced by Keck Biotechnology Resource Laboratory (Yale).

Replicates

All experiments were done with at least two biological replicates. Experimental replicates are indicated in the corresponding description for each figure, as appropriate.

### Results

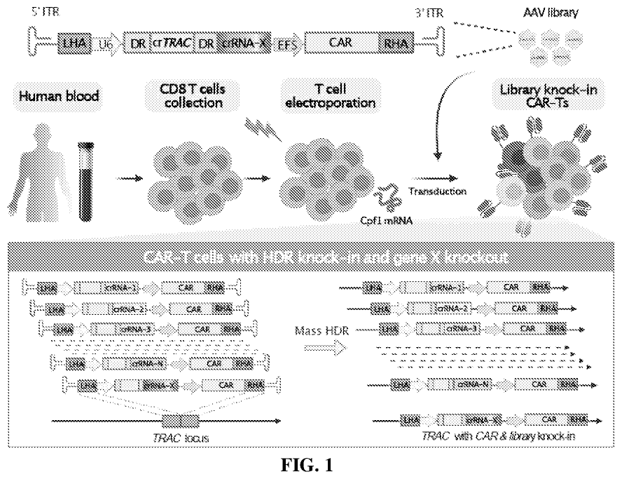

Currently, there is no approach for high-throughput CAR-T engineering due to various challenges. The CLASH system was therefore built based on the advantageous features of AAV as well as the Cas12a/Cpf1 gene editing system. Taking the advantage of AAV vectors, three components were encoded into the transgene: homology-directed repair (HDR) arms for targeted knock-in, CAR-expression cassette, and Cas12a/Cpf1 CRISPR RNA (crRNA) expression cassette for genetic manipulation (FIG. 1). While HDR can be used to target any place in the genome, the TRAC locus was first targeted for clinically-relevant CAR knock-in. The base of the CAR-expression cassette can be standardized so that all variants are directly comparable for their phenotypes, such as persistence. The crRNAs can be a single element, or can be readily engineered in a pooled manner by simple molecular cloning. Due to the advantages of transactivating RNA (tracrRNA)-independence, multiple crRNAs can be engineered to be expressed under the same polymerase III promoter. An AAV vector (CLASH AAV vector, CLASH vector for short) expressing three components was engineered: (1) an anti-CD22 CAR construct with CD22-scFv, a transmembrane domain (TM), and a signaling domain (4-1BB, CD3z) (CAR22 for short); (2) a constitutive crRNA targeting the 5′ end of the first exon of TRAC to facilitate knockin; and (3) a wildcard crRNA cassette separated from crTRAC by Cas12a/Cpf1 direct repeats (DR) to test virtually any number of crRNAs against any set of genes. All of these components were flanked by the 5′- and 3′-TRAC HDR arms so that they could be simultaneously knocked into the same position (FIG. 1). The CLASH AAV vector thereby provided three distinct functions in one setting: knock-in into the TRAC locus, CAR expression, and targeted mutagenesis.

To permit massively parallel CAR knock-in human primary T cells, a workflow for CLASH-mediated human CAR-T cell engineering was developed and optimized (FIG. 1). In this workflow, Cas12a/Cpf1 mRNA is first delivered into human primary CD8 T cells by electroporation, then transduced with an AAV6 encoding CLASH vector or library. To test CLASH's CAR generation efficiency, the on-target integration of CAR into T cells was measured by FACS at five days after transduction. By staining for CD3 which forms a surface complex with TCR, TRAC knockdown efficiency (CD3⁻) was determined to be >60%, with on-target integration of CAR22 (CD3⁻CAR22⁺) at 37.4% and 51% in donor 2 and donor 3 CD8 T cells, respectively.

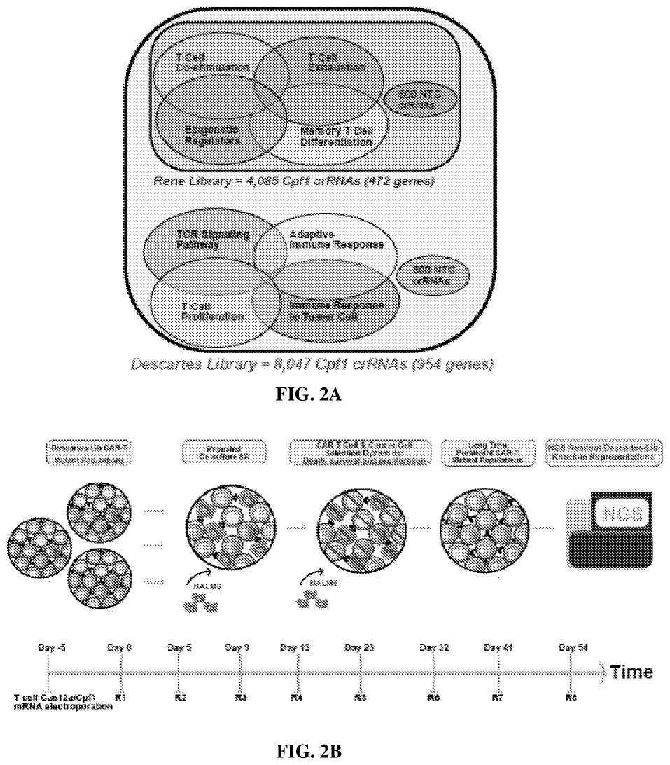

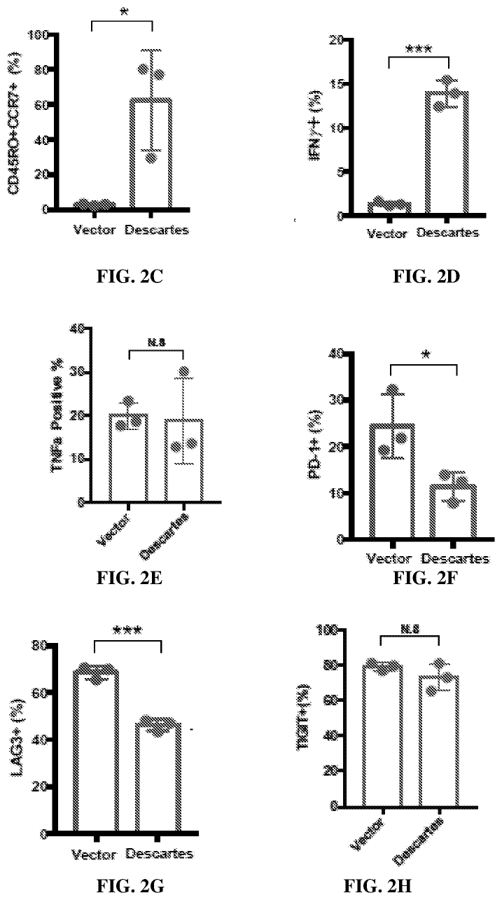

In order to achieve massively parallel CAR-T engineering (CAR-T mass engineering) with immunologically relevant targets, two Cas12a/Cpf1 guide RNA libraries were designed to diversify the wildcard crRNA position with targeted mutagenesis. The first library, Descartes, contained 8,047 crRNAs, targeting 954 immune genes (see Table 2) with 8 crRNAs per gene for most genes, and 1000 non-targeting controls (NTCs) (FIG. 2A). The immune genes were chosen as a superset from gene sets implicated in T cell exhaustion (Wherry et al., 2007), epigenetic regulators (Arrowsmith et al., 2012), T cell co-stimulation, memory T cell differentiation, T cell receptor signaling pathway, adaptive immune response, immune response to tumor cell, T cell proliferation, and TET2 which is also an epigenetic regulator. Similarly, a smaller library, Rene, was also designed that contained 4,085 crRNAs targeting a more refined set of genes (Table 3). All crRNAs were scored for selection using Deep Cpf1 (Kim et al., 2018) to enhance potential gene editing efficiency and reduce potential off-target effects. These libraries were cloned into the CLASH AAV vector. The library compositions were verified by next-generation sequencing (NGS) using vector-specific primer readout.

To test (i) whether the entire CLASH construct was integrated into TRAC locus in the human T cell genome, and (ii) whether the scale of the knock-in was achieved for multiple constructs in the same pool of T cells, specific primers flanking the genomic regions outside the 5′- and 3′-HDR arm were used to amplify the genomic regions rather than the AAV donor, and sequence the inserted region. The Sanger sequencing results showed that, first, the designed knock-in regions were indeed in the genomic DNA; and second, there was clear sequence degeneracy in the crRNA wildcard region, indicating that diverse crRNAs exist in the targeted pool of human T cells. Sanger sequencing results of Descartes-Lib AAV plasmid and pooled Descartes-Lib CD22 CAR-T cell genomic DNA at TRAC locus showed degenerated at the library site in both Descartes-Lib AAV plasmid pool and Descartes-Lib CLASH knock-in genomic DNA pool, but not in either vector controls. With the successful pooled knock-in observed, this CLASH system thus provided a platform for high-throughput generation of defined genomic-integrated CARs in custom-defined library scale in human T cells.

### Example 2: CLASH Mediates High-Throughput Engineering of Pooled CAR-T Variants and Selection in Long-Term Co-Culture

### Materials and Methods

CLASH time-course dynamics of long-term CAR-T co-culture

T cells were infected with vector or Descartes AAV6 after electroporation with NLS-LbCpf1mRNA. After electroporation for 5 days, the percentage of positive CAR-T cells was determined by staining with CD3 and CAR specific antibodies. 2×10⁶ positive CAR-T cells per replicate were used with a minimal representation of 20× transduced cells per crRNA. T cells were co-cultured with NALM6 at low E:T (0.2:1) ratio. After clearance of NALM6, new rounds of stimulation were performed until vector CAR-T cells were exhausted. After each round of stimulation, the T cells were harvested and frozen in liquid nitrogen. Genomic DNA (gDNA) was isolated by DNA purification kit (Qiagen).

CLASH CAR-T Coculture Time-Course Readout

After each round of stimulation, the T cell genomic DNA (gDNA) was isolated by DNA purification kit (Qiagen). The crRNA library readout was performed using a two-step PCR strategy, where the first In-Out PCR was used to amplify out the integrated fragments from gDNA and the second PCR adds appropriate sequencing adapters to the products from the first PCR. For the first round PCR, the thermocycling parameters were 98° C. for 1 min, 20 cycles of (98° C. for Is, 60° C. for 5 s, 72° C. for 25 s), and 72° C. for 2 min. In each PCR reaction, 2 μg of total gDNA was used for in vitro and 5 ul DNA extraction solution was used for in vivo. A total of 3-4 reactions was used to capture the full representation of the library. PCR products for each biological sample were pooled and used for amplification with barcoded second PCR primers. For the second round PCR, the thermocycling parameters were 98° C. for 1 min, 28 cycles of (98° C. for Is, 61° C. for 5 s, 72° C. for 10 s), and 72° C. for 2 min. Second PCR products were pooled and then normalized for each biological sample before combining uniquely barcoded separate biological samples. The pooled product was then gel purified from a 2% E-gel EX (Life Technologies) using the QIAquick Gel Extraction Kit (Qiagen). The purified pooled library was then sequenced with HiSeq or NovaSeq systems (Illumina).

Flow Cytometry

All antibodies for flow cytometry were purchased from Biolegend. All flow antibodies were used at 1:200 dilutions for staining unless otherwise noted. For surface staining, cells were stained with surface marker antibodies in the staining buffer of 2% FBS in PBS on ice for 30 min. Samples were washed twice with 2% FBS in PBS before analysis. For CAR staining, the CD22BBz CAR transduced T cells were incubated with 0.2 ug CD22-Fc (R&D system) in 100 uL staining buffer for 30 mins, and then stained with PE-IgG-Fc (Biolegend). For intracellular cytokine staining analysis, CAR⁺ T cells and NALM6 were plated at 1:1 E:T ratio in 96-well plate (Corning) and 0.2 μl per test Brefeldin A Solution (1000×, Clone BFA, BioLegend) for 5 h. After incubation, intracellular cytokine staining was performed using the BD Cytofix/Cytoperm™ Fixation/Permeabilization Solution Kit (BD) according to the manufacturer's instruction using the following antibodies from BioLegend: PerCP/Cyanine5.5 anti-human/mouse Granzyme B (clone QA16A02), FITC anti-human TNFα [clone MAb 11], APC anti-human IFNγ (clone B27).

Standard Statistical Analysis

All statistical methods are described in the corresponding description of the figure. The P values and statistical significance were estimated for all analyses. The unpaired, two-sided, Mann-Whitney test was used to compare two groups. One-way ANOVA, two-way ANOVA, Dunnett's multiple comparisons test, Tukey's multiple comparisons test was used to compare multiple groups. Data between two groups were analyzed using a two-tailed unpaired t-test. Multiple t-test using the Holm-Sidak method was used for multiple group comparison. Different levels of statistical significance were accessed based on specific p values and type I error cutoffs (0.05, 0.01, 0.001, 0.0001). Data analysis was performed using GraphPad Prism v.8. and RStudio.

### Results

CAR-T cell therapy is limited by poor T cell expansion and persistence, especially under chronic exposure to viral or tumor antigens that can result in T cell dysfunction (Savoldo B., et al., Clin Invest., 121(5):1822-6 (2011); Shin H., and Wherry E J., Curr Opin Immunol., 19:408-415 (2007)). Thus, the CLASH system was used to rapidly identify more persistent forms of CAR-T. In a small-scale experiment, it was observed that repeated co-culture of CAR-T cells with antigen-specific tumor cells at lower effector:tumor (E:T) ratio significantly promoted T cell differentiation with decreased T central memory cells population (CD45RO⁺CD62L⁺) and decreased capacity to express IFNγ and TNFα with continuous exposure, after each round of co-culture. An in vitro long-term culture system was designed to identify genes whose perturbation can increase the longevity and cytotoxicity of CAR-T cells with chronic antigen exposure (FIG. 2B). With the pipeline established above, AAV-CLASH Descartes-Lib was used to rapidly generate pools of TRAC knock-in human CAR-T variants. The empty CLASH vector was used to generate control knock-in CAR-T cells that would otherwise be identical but without additional mutagenesis. After transduction with vector or Descartes-Lib AAV6, the control or pool mutant CAR-T cells were repeatedly co-incubated with NALM6 cells at E:T ratios of 0.2 for 54 days, and a fraction of them were collected at each round for genomic DNA prep and deep sequencing. The long-term culture was performed with three independent series, so that each CLASH Descartes CAR-T pool had a matched time-series.

Initially (at day 0), the vector and Descartes-Lib transduced CAR-T cell pools showed similar immune phenotypes. During tissue culture, dynamic alterations of the cancer cell to CAR-T cell ratios after multiple rounds of co-culture were observed, indicative of selection within the pools (FIG. 2B). After the last round of stimulation at day 54, vector CAR-T lost killing ability, with 90.6% NALM6 cells (CD8⁻CAR22⁻) remaining, whereas Descartes-Lib CAR-T cells had drastically more efficient tumor cell clearance compared with vector CAR-T cells, where only 2.7% NALM6 cells (CD8⁻CAR22⁻) remained. The percentages of central memory CAR-T cells in vector and Descartes-Lib CAR-T cells were not different prior to stimulation. However, the Descartes library significantly prevented T cell terminal differentiation at day 54 as indicated by the significantly increased CD45RO⁺CCR7⁺ cell population in the pool (FIG. 2C). To investigate whether Descartes-Lib pool CAR-T cells maintain cytotoxicity after long term co-culture, intracellular IFNγ and TNFα were measured by FACS after re-stimulation with specific antigen for 5 hours. Compared to vector CAR-Ts, the Descartes-Lib CAR-T cell pools showed higher levels of IFNγ at the endpoint (d54), but not TNFα (FIGS. 2D-2E). The Descartes-Lib CAR-T cell pools showed reduced T cell exhaustion with diminished surface levels of PD-1 and LAG3, but no change in TIGIT (FIGS. 2F-2H). The observation in the differences between pool mutant vs. wildtype CAR control at the gross population level indicated that at least subsets of mutant variants in the Descartes-Lib pool were contributing to the shifts of various phenotypes of these CAR-T cells.

Identification and Validation of Candidate CAR-T Variants in Long-Term Co-Culture

To determine the actual composition and dynamics of the variants, the library readout method was optimized and NGS was performed on all the experimental replicates at all collection time points during the entire time course of CAR-T: cancer cell co-culture, which determined the crRNA representation in the Descartes library across all samples. Correlation analysis of the crRNA representations in the genomic readouts of Descartes-Lib CLASH knock-in CAR-T pool was performed. Pearson correlations of crRNA library representation across time points were calculated based on log 2 rpm values. It was observed that the crRNA representation for samples collected at each time point naturally clustered and were more correlated with each other than with samples at other time points, suggesting a high degree of consistency. The crRNA representation of corresponding samples along the time points were also more similar to the matched samples at other timepoints than the other two non-matching samples, displaying a tile pattern in the correlation heatmap, showing the consistency of matched samples along the time course trajectory, and thereby demonstrating high level of technical reproducibility. In all three replicates, the diversity of the library reduced over time and the CAR-T library pool became increasingly dominated by a smaller fraction of crRNAs over time, as shown in the cumulative distribution function (CDF) plot, indicative of a time gradient in this process.

### Example 3: Identification and Validation of Genes Whose Loss of Function Enhance Persistence of CAR-T Variants

### Materials and Methods

Primary and Dynamic Time-Course of Cas12a/Cpf1 crRNA Library Representation Analysis

Raw read counts from each sample was converted to reads per million (rpm), then log 2 transformed for certain analyses. Pearson correlations for heatmaps were calculated using the cor function in R, and empirical cumulative distribution function was computed and plotted using stat_ecdf from ggplot. RIGER and false-discovery rate (FDR) based criteria were used to determine top candidate genes. For RIGER analysis of CRISPR screens, read count tables were used to calculate log fold changes for T cell samples collected from each day vs day 0 samples to score and rank sgRNAs, with ties in rank broken by random order. These data were then used as input to a Java-based implementation of RIGER (github.com/broadinstitute/rigerj) to generate P values and gene rankings based on consistent enrichment across multiple sgRNAs for identification of candidate genes (Shalem O., et al., Science, 343:84-87 (2014)). Both the second highest-ranking sgRNA and the weighted sum scoring methods were used for computation of gene rankings. For FDR based analysis, a crRNA was determined to be statistically significant if it was enriched using a false-discovery rate (FDR) threshold of 1.0% or 5.0% based on the abundances of all non-targeting controls. Both primary and dynamic time-course crRNA representation analyses were performed across multiple different in vitro and in vivo screening readouts. Custom R scripts were used, including for Venn diagrams and other visualizations, heatmaps, and statistical analyses

Generation of CD22 CAR-T with Individual Gene Perturbation

The individual crRNAs were cloned into the CLASH vector plasmid by linearization with BbsI digestion and quick ligation kit (NEB). The ligated products were transformed into Stb13 competent cells by heat shock at 42° C. for 90 s. A single clone was picked and the plasmid was isolated by mini prep kit (Qiagen). The plasmid sequence was confirmed by Keck Biotechnology Resource Laboratory (Yale).

T7E1 Assay

Five days after electroporation and AAV transduction, positive CAR-T cells were stained and sorted by BD FACSAria II. The genomic DNA was extracted using QIAamp DNA Blood Mini Kit (Qiagen). PCR amplification of the genomic regions flanking the crRNAs was performed using the appropriate primers. Using Phusion Flash High Fidelity Master Mix (Thermo Fisher), the thermocycling parameters for PCR were 98° C. for 2 min, 35 cycles of (98° C. for 1 s, 60° C. for 5 s, 72° C. for 15 s) and 72° C. for 2 min. The PCR amplicons were then used for T7E1 assays according to the manufacturer's protocol. Statistical significance was assessed by two-sided unpaired Welch's t-test.

Nextera Library Prep and Amplicon Sequencing

The PCR products described from the T7E 1 experiments were used for Nextera library preparation following the manufacturer's protocols (Nextera XT DNA Library Preparation Kit, Illumina). Briefly, 1 ng of purified PCR product was fragmented and tagged using the Nextera Amplicon Tagment Mix according the to manufacturer's recommendations, followed by limited-cycle PCR with indexing primers and Illumina adaptors. After this amplification, DNA was purified and sequenced using 100-bp paired-end reads on an Illumina HiSeq 4000, Novaseq or equivalent. For indel quantification, reads were mapped to expected amplicon sequences using BWA-MEM with the −M option. 100 bp reads from the SAM file that fully mapped within a +/−75 bp window of expected cut site within the amplicon were then identified (soft-clipped reads discarded). Indel reads were then identified by the presence of “I” or “D” characters within the CIGAR string. Cutting efficiency was quantified as percentage of indels over total (indel plus wild-type) reads within the defined window.

CLASH CAR-T crRNA Screen Processing

Raw single-end fastq read files were filtered and demultiplexed using Cutadapt. Reads were demultiplexed for the barcodes included in the forward readout PCR primers. To identify and remove extra sequences immediately upstream of the crRNAs, the following settings were used: cutadapt -g TAATITCTACTAAGTGTAGAT (SEQ ID NO:12,136) -e 0.1-m 19--discard-untrimmed. The 20 bp crRNA sequences were then mapped the designed Descartes library sequences using a bowtie index generated using the bowtie-build command in Bowtie 1.2.2. Mapping used the following settings: bowtie -v 1 -m 1, and the number of reads that had mapped to each crRNA within the library was quantified. This processing was used across multiple different in vitro and in vivo CLASH crRNA library readouts, as well as for the MIPs crRNA representation readout.

CLASH Time-Course of In Vivo CAR-T Representation in a Cancer Model

NOD.Cg-Prkdcscid Il2rgtm1Wjl/SzJ (NSG) mice were purchased from the Jackson Laboratory and bred inhouse. NSG mice between 6-8 weeks old were used and inoculated with 5×10⁵ NALM6-GL cells intravenously. Mice were randomly assigned to different groups prior to treatment. 2×10⁶ vector CAR-T cells or Descartes CAR-T cells were infused back to mice after 3 days. NSG mice was euthanized at days 7, 11 and 14. Spleen and bone marrow were collected immediately. Red blood cells were lysed by incubation with ACK (Ammonium-Chloride-Potassium) Lysis Buffer (Thermo Fisher) for 2 mins. After washing, the cell surface markers were labeled as described for FACS and assessed by BD FACSAria II. CAR positive T cells were sorted and the genomic DNA was extracted using QuickExtract DNA extraction solution (Lucigen).

MIPs Library Selection and Cloning

The MIPs library contains 56 crRNAs, targeting 7 top hit genes from the validation, with approximately 8 crRNAs per gene. The 56 crRNAs were selected from the Descartes library. To generate the MIPs AAV vector, the pre-mixed crRNAs were cloned into the PXD60 plasmid by linearization with BbsI digestion and quick ligation. The MIPs library products were transformed into high-efficiency competent cells (Endura) by electroporation. An estimated crRNA library coverage of >300× (16,800 colonies) was observed after electroporation by colony counting. All the bacteria were harvested in pool. Plasmid library was purified using EndoFree Plasmid Maxi Kit (Qiagen). The representation of crRNAs in library plasmid was verified by NGS. MIPs library transduction was performed similarly to the library scale AAV transduction. Three reactions were set for the MIPs library electroporation.

MIPS Probe Design

MIPS probes were designed according to previously published protocols (website: github.com/shendurelab/MIPGEN), then processed by a customized selection algorithm. 107 MIPs probe were designed using MIPgen. Briefly, the 77 bp flanking the predicted cut site of each crRNA of all 56 unique crRNA were chosen as targeting regions, and the bed file with these coordinates was used as an input. These coordinates contained overlapping regions, which were subsequently merged into 31 unique regions. Each probe contains an extension probe sequence, a ligation probe sequence, and a 6-bp degenerate barcode (NNNNNN) for PCR duplicate removal. A total of 107 MIP probes were designed (SEQ ID NOs:12,139-12,245), covering a total amplicon of 5,675 bp. The statistics for the MIP target size were as follows: minimum, 154 bp; maximum, 331 bp; mean, 183 bp; median, 163 bp. Each of the MIPs was synthesized using standard oligo synthesis (IDT), normalized, and pooled.

MIPS Target-Capture Sequencing

After 10 days of electroporation, CAR positive cells from the MIPS library and control group were sorted by FACS (BD), then CAR-T cells genomic DNA was isolated by DNA purification kit (Qiagen). The experimental workflow was done following standard protocols. In brief, 50 -100 ng of high quality, non-fragmented genomic DNA was used for hybridization. After gap filling and ligation, circularized DNA molecules were used as template in PCR with universal primers complementary to the linker sequence. Then, sample-specific barcode sequences and Illumina adaptors were introduced during the PCR amplification step. After this amplification, DNA was purified and sequenced using 100-bp paired-end reads on an Illumina HiSeq 4000, Novaseq or equivalent.

MIPS Data Analysis

For MIPS library crRNA representation in plasmid and samples, standard screen processing and mapping was performed as described above using a subset library with the MIPs crRNAs. For mutation-based MIPs target-capture sequencing analyses, raw pair-end fastq read files were first mapped to the reference hg38 Homo sapiens genome assembly and sorted using BWA and SAMTools. For coarse filtering, reads near target crRNA regions were selected (+/−1000 bp) using BEDTools and then indexed and used for variant calling using SAMTools and Varscan v2.4.1. with the parameters pileup2indel --min-coverage 1 --min-reads2 1 --min-var-freq 0.001 --p-value 0.05. Generated VCF files were then used for fine mapping to crRNA cutting regions.

To define cutting regions, crRNAs were first collapsed based on whether their cutting sites were contiguous within or equal to 16 bp. For mapping, crRNA cutting regions were defined as +/−8 bp from max/min of cutting sites for collapsed crRNAs (non-collapsed crRNAs will therefore have 16 bp windows, but collapsed crRNAs will be larger). For insertions, the variant position point in genome was kept as defined by VCF output. For deletions, the variant position point in genome was adjusted to reflect the center point of the deletion. Variants were mapped to crRNA cutting regions based on the above. Further downstream analyses including those comparing cutting efficiency with crRNA representation were based on collapsed crRNA references. For MIPs crRNA representation in the library and associated cutting efficiency bar plots, crRNAs with near 0 reads in library sequencing (PRDM-cr6 and PRDM-cr7 for MIP1, PRDM-cr1 for MIP3) were removed for visualization—but were included in all other analyses and statistical tests. For analyses including MIPs cutting efficiency normalized by library representation, mean summed variant frequencies for each target region were divided by a normalization factor defined as: (mean library reads)/median (mean library reads). Statistical tests and visualizations were performed using custom R scripts.

### Results

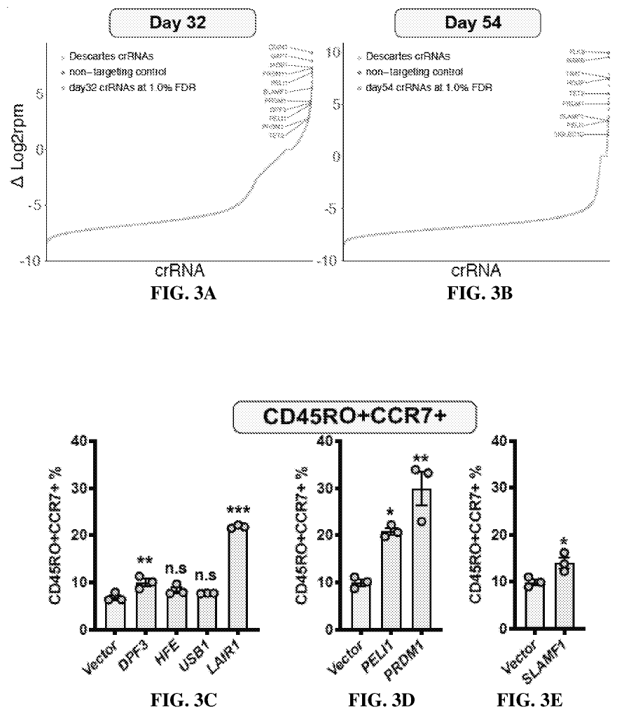

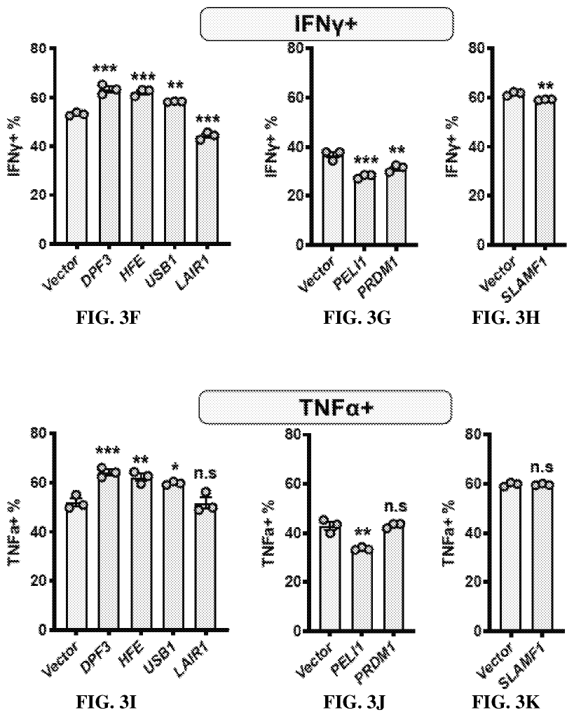

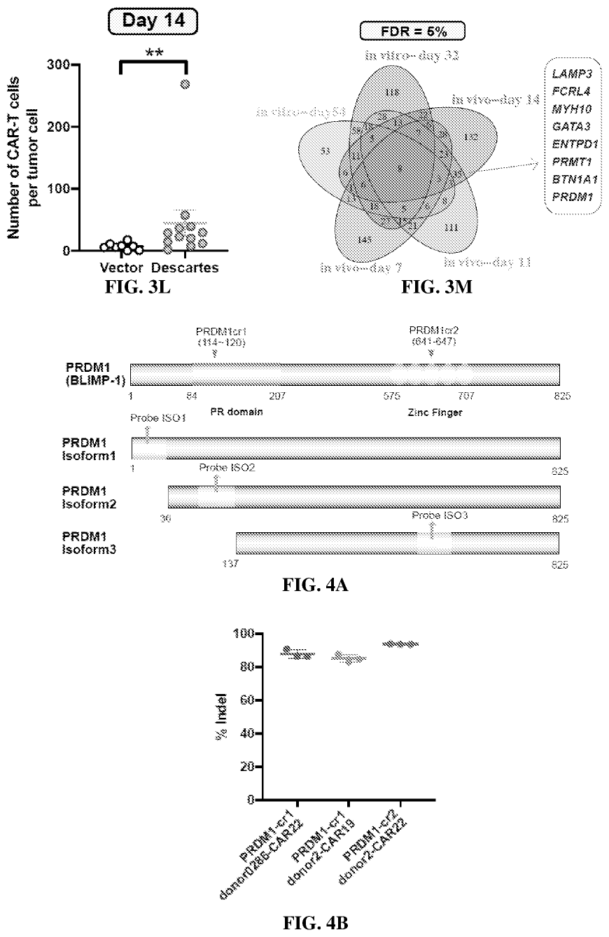

A series of analyses was performed to identify which variants of the CAR-Ts in the Descartes pool are more persistent and potentially have enhanced effector function. From an overall time-course heatmap of all crRNAs, it was observed that the majority of crRNAs diminished over time, and that most of the crRNAs became depleted by day 32. However, distinct sets of crRNAs emerged as persistent clusters at varying degrees. To identify enriched hits, examination of the time course at a late time point (e.g., d32) and a terminal time point (d54) for individual crRNAs and individual genes was performed. A fraction of the crRNAs were highly enriched at day 32 at a false-discovery rate (FDR) of 1.0%, and fewer crRNAs were enriched at day 54. Comparison of day 32 or day 54 crRNA abundance to the ground zero baseline (day 0) revealed rapidly decaying distributions of persistent crRNAs, with a small number of enriched crRNAs over baseline (FIGS. 3A-3B). Examples of these crRNAs showed varying degrees of persistence up to various time points. Representative highly enriched crRNAs target several potential candidate genes such as PRDM1/BUMP-1, DPF3, SLAMF1, TET2, HFE/HLA-H, and PEL11. RIGER analysis also revealed a number of similar high-ranked candidate genes including PRDM1, DPF3, SLAMF1, HFE, and PEL11.

To examine the behaviors of individual crRNAs targeting the same gene across three replicates over time, crRNAs were internally compared against each other and externally to the putatively neutral behavior, the mean and 99% confidence interval (CI) of the 1,000 NTCs, and were visualized on the same plot for each gene. Certain known T cell exhaustion surface markers (PDCD1, HAVCR2/TIM3), transcription regulators previously implicated in T cell function (TET2, NR4A2), and under-characterized candidates identified from the analyses of the time-course (PRDM1, DPF3, PEL11 and LAIR1) were examined. While the NTC mean had a median decay of 20 days, distinct crRNAs, representing distinct CAR-T variants were observed to persist at a higher level at d20 or later time points (d32, d41 or d54). TET2 has been previously identified as a key factor suppressing CAR-T expansion and persistence in vivo (Fraietta J A, et al., Nature, 558:307-312 (2018). One TET2 mutant CAR-T variant, as well as multiple mutant variants of NR4A2, PRDM1, DPF3, PEL11 and LAIR1 showed enhanced persistence as compared to NTC CAR-Ts.

Based on their time course dynamics, the following seven candidate genes previously not documented in CAR-T function, were further investigated: DPF3, HFE/HLA-H, LAIR1/CD305, USB1, PEL11, PRDM1/BLIMP-1 and SLAMF1/CD150. To test whether these genetic variants of CAR-T cells have enhanced anti-tumor phenotypes, individual CAR-T variants were first re-generated using single AAV-CLASH vectors encoding CAR22 and top-rank crRNAs targeting each of these genes. Next the cutting efficiency was measured by T7E1 endonuclease assay, and then by NGS, finding that all genes except LAIR1 showed high efficiency gene editing (DPF3, HFE, USB1, PEL11, PRDM1 and SLAMF1 achieved greater than 80% indels) where most of them were out-of-frame. The memory cell population was measured by flow cytometry, and it was observed that the central memory T cell population was increased in DPF3, LAIR1, PEL11, PRDM1 and SLAMF1 mutant CAR-Ts as compared to vector control CAR-T (FIGS. 3C-3E). Quantification of intracellular IFNγ and TNFα levels showed that DPF3, HFE and USB1 edited CAR-T cells have higher levels of IFNγ and TNFα after stimulation with NALM6 for 5 hours (FIGS. 3F-3K). PELI1 mutants exhibited lower levels of both IFNγ and TNFα; PRDM1 and LAIR1 mutants exhibited lower levels of IFNγ but not TNFα. SLAMF1 mutant exhibited moderate to little change in the levels of IFNγ and TNFα relative to the vector (FIGS. 3F-3K).

In order to determine whether the ability of gene editing by crRNAs correlated with their screen performance in the CLASH experiment, a CLASH-MIPS (Molecular Inversion Probe Sequencing) experiment was performed in T cells. In this experiment, the crRNA abundance was measured by genome-integrated crRNA library readout, and the actual gene editing efficiency of individual crRNAs was measure by MIPS using biological triplicates. The results were then compared with the crRNAs' screen performance in the CLASH experiment. A minipool of 56 crRNAs targeting 7 top candidate genes was designed as above and cloned into the CLASH vector and CLASHed human CD8 T cells (by mRNA electroporation and AAV6 transduction as above), to generate a CAR-T minipool. The size of the minipool was determined considering (1) the sensitivity of MIPS to measure genomic variants; (2) the ability of MIPS to capture actual genome editing events in each of the specific crRNA target sites, with dilution of editing events in a pooled manner; and (3) the relative gene editing challenges in T cells. Library readout successfully mapped the crRNA abundance of the minipool. The ability of individual crRNAs' gene editing (measured by MIPS) was compared to their screen performance in the CLASH-Descartes experiment using the d32 data, as a balanced timepoint when a substantial time period has elapsed for selection but not to a stage when the majority of crRNAs are lost (e.g., d54). While the different crRNA sets of individual genes showed varying strengths of correlation between gene editing efficiency in a pooled manner (MIPS) and screen performance (CLASH), considering all genes/all crRNAs measured, the overall gene editing efficiency (MIPS) significantly correlated with mean screen performance (CLASH). This significant correlation also holds regardless of whether the gene editing efficiency is normalized by crRNA abundance or not. These data indicate that the screen performance of crRNAs significantly correlates with their capacity for gene editing in T cells, and, together with the fact that the majority of the crRNAs tested individually showed high gene editing efficiency, the crRNAs enriched in the screen largely represent true cutters and screen performers for the top candidate genes.

CLASH-Mediated Human CAR-T Variant In Vivo Selection in a Cancer Model

To further identify which CAR-T variants have better anti-tumor phenotypes, a time-course in vivo CLASH-Descartes experiment was performed to identify genetic perturbations that can increase CAR-T persistence in a leukemia mouse model. Leukemia induction was performed by transplanting NALM6-GL cells into NSG mice. Three days post induction, Descartes-Lib CAR-T variants were infused into mice by adoptive transfer. Bone marrow and spleen samples were collected at day 7, day 11, and day 14. A higher CAR-T: cancer cell ratio in recipients of Descartes-Lib CAR-T cells was observed at day 14 after CAR-T infusion (FIG. 3L). The crRNA library representation of the Descartes library in these in vivo samples was then readout and the deep sequencing data was analyzed to identify enriched crRNAs in day 14 in vivo samples compared to the pre-injection day 0 CAR-T cell pool. A fraction of crRNAs were observed to be highly enriched in day 14 in vivo samples at FDR 1.0%, for example TNFRSF21, TBX21, SIRT7, GPR65, PRDM1 and PRDM4. The results from in vivo CLASH were then compared to the in vitro long-term co-culture CLASH experiment, using enriched gene sets from independent time points from both in vitro and in vivo. Substantial overlap of enriched genes between in vitro day 32, in vitro day 54, in vivo day 7, in vivo day 11, and in vivo day 14 (FIG. 3M). Eight genes were significantly enriched across all these five gene sets at an FDR of 5%, including LAMP3, FCRL4, MYH10, GATA3, ENTPD1, PRMT1, BTN1A1 and PRDM1 (FIG. 3M).

### Example 4: PRDM1 Mutant CAR-T Cells Exhibit Increased Memory Cell Population, Stronger Antigen-Stimulated Proliferation, and Maintained Cytotoxicity

### Materials and Methods

CAR-T Purification

CAR positive T cells were purified by streptavidin microbeads (Miltenyi Biotec). Briefly, 1×10⁷ cells were suspended in 100 μL labeling buffer, and then incubated with 1 μg Pierce™ Recombinant Biotinylated Protein L (Thermo Fisher) and 10 μl FcR Blocking Reagent (Miltenyi Biotec) for 15 minutes at 4° C. Cells were washed to remove unbound protein and labeled with 10 μL streptavidin microbeads for 15 minutes on ice. After wash, the suspension was loaded onto MACS column for separation according to manufacturer's protocol (Miltenyi Biotec).

Kill Assay (Co-Culture)

GFP and firefly luciferase genes were stably transduced into the NALM6 cell line using a lentivirus. 2×10⁴ NALM6-GL cells were seeded in a 96 well plate. The engineered CAR-T, vector CAR-T, or normal CD8 T cells were co-cultured with NALM6-GL at indicated E:T ratios for 24 hours. To test the luciferase expression in NALM6-GL, 150 μg/ml D-Luciferin (PerkinElmer) was added into each well. After 10 minutes, luciferase intensity was measured by a plate reader (PerkinElmer). Direct tumor cell killing was quantified by luminescence. Luminescence units were normalized to control (NALM6-GL without any effector cells, LUC). The calculation formula is: % Cytotoxity=100−LU^{sample}/LU×100.

Western Blot

Cells were lysed by ice-cold RIPA buffer (Boston BioProducts) containing protease inhibitors (Roche, Sigma) and incubated on ice for 30 minutes. Protein supernatant was collected after centrifuging at 13,000 g at 4° C. for 30 minutes. Protein concentration was determined using the BCA Protein Assay Kit (Thermo Fisher). Protein samples were separated under reducing conditions on 4-20% Tris-HCl gels (Bio-Rad) and analyzed by western blotting using primary antibodies PRDM1/Blimp-1mouse mAb (R&D 1:1000) followed by secondary anti-mouse HRP antibodies (Sigma-Aldrich, 1:10,000). Blots were imaged with an Amersham Imager 600.

Immunoprecipitation

PRDM1 deficient CD22 CAR-T cells or vector cells were lysed by ice-cold RIPA buffer with protease inhibitor cocktail (Roche) for 30 minutes. Immobilized BLIMP1/PRDM1 Antibody (CST) was added on the NHS-activated agarose beads according to manufacturer's protocol (Thermo Fisher). Cell lysates were incubated with beads at 4° C. and rotated for 1 hour. The supernatant was discarded after spin down for 1 minute at 3,000 RPM. Beads were washed 4 times with ice-cold TBS and boiled for 10 minutes with SDS loading buffer. Protein samples were loaded on 4-20% Tris-HCl gels (Bio-Rad).

In-Gel Digestion and Mass Spectrometry

Protein-containing gel slices were digested with trypsin overnight. The resulting peptide mixtures were extracted from the gel and run directly on an Orbitrap Velos instrument (Thermo Fisher Scientific) with 120-min liquid chromatography (Buffer A: 0.1% formic acid water; Buffer B: 0.1% formic acid MeCN; Gradient: 0% to 95% buffer B; flow rate: 0.1 μl/min) and tandem mass spectrometry (LC-MS/MS) using a standard TOP20 method procedure. Briefly, MS1 m/z regions for 395-1,600 m/z ions were collected at 60 K resolving power and used to trigger MS/MS in the ion trap for the top 20 most abundant ions. Active dynamic exclusion of 500 ions for 90 s was used during the LC-MS/MS method. Peptides were eluted with 300 nl/min flow rate using a NanoAcquity pump (Waters). Samples were trapped for 15 min with a flow rate of 2 μl/min on a trapping column 100-micron ID packed for 5 cm in-house with 5 μm Magic C18 AQ beads (Waters) and eluted with a gradient to 20 cm 75-micron ID analytical column (New Objective) packed in-house with 3 μm Magic C18 AQ beads (Waters).

Mass spectra were analyzed using Scaffold Q+/Q+S Version 4.0 against Uniprot database. A mass deviation of 20 ppm was set for MS1 peaks, and 0.6 Da was set as maximum allowed MS/MS peaks with a maximum of two missed. Maximum false discovery rates (FDR) were set to 0.01 both on peptide and protein levels. Minimum required peptide length was five amino acids.

CLASH-PRDM1 Genome-Wide AAV Integration Library Preparation

In order to perform genome-wide profiling of the CLASH-PRDM1 off-target integration events, a method modified from GUIDE-Seq (Tsai, S Q. et al., Nat Biotechnol., 33:187-197 (2015)) was developed. Briefly, CAR-T cells' genomic DNA (gDNA) was extracted after AAV transduction for 9 days. The adapter was made by annealing the Miseq common oligo with the sample barcode oligo (A01-A06) in TE buffer. The annealing program was set from 95° C. for 1 s; slow ramp down (approximately −2° C./min) to 4° C. The gDNA was fragmentated to ˜1,500 bp by using S220 focused-ultrasonicator (Covaris). End-repair and dA-tailing was performed by using NEBNext® Ultra™ End Repair/dA-Tailing Module (NEB) according to manufacturer's protocol. The repaired DNA was ligated with annealed adaptor by using T4 DNA ligase at room temperature for 1 hour. The sample was cleaned by using 0.9×SPRI (Beckman). The genome-wide off-target integration library was performed using a two-step PCR strategy with streptavidin beads purification, where the first PCR was used to bait-prey the fragments with integrated CAR gene from gDNA using biotinylated primer and the biotinylated fragments were purified by using streptavidin beads (Thermo). Next, second PCR added appropriate sequencing barcodes to the products from the first PCR. The Q5 was used for PCR (NEB). The thermocycling parameters for the two-round PCR were 98° C. for 30 s; 7 cycles of 98° C. for 10 s, 70° C. (−1° C./cycle), 72° C. for 1 min; 13 cycles of 98° C. for 10 s, 63° C. for 30 s, 72° C. for 1 min; 72° C. for 1 min, and hold at 4° C. PCR products for each biological sample were normalized and pooled. DNA less than 1 kb was selected. The sample was sequenced by using custom sequencing primers with Miseq (2×300 bp pair-end).

CLASH-PRDM1 Genome-Wide AAV Integration Data Analysis

Paired-end reads were processed using Cutadapt 3.2, BWA 0.7.17, and SAMtools 1.12 to identify off-target integration events. R2 reads containing TRAC elements were first trimmed and selected using cutadapt -GGTITACTCGATATAAGGCCTTGA (SEQ ID NO:12,137) -e 0.2 -m 20 --discard-untrimmed. Then, R2 reads containing ITR reads were trimmed and selected using cutadapt -GAAAGGTCGCCCGACGCCCGG (SEQ ID NO:12,138) -e 0.2 -m 20 -O 15 --discard-untrimmed. The ITR trimmed reads were then mapped to the Homo sapiens genome assembly GRCh38 (hg38) using BWA. In order to see where mapped reads target the genome without double counting at positions, reads were converted to single base pair coordinates using the starting positions and then converted to bedGraph format. Reads were normalized for sequencing depth using the R2 reads containing TRAC elements. Visualizations were generated using Integrative Genomics Viewer 2.9.2 and R packages including GenomicAlignments and ggbio.

TRAC Integration PCR

The gDNA was extracted after CLASH-Vector AAV transduction for 9 days. Using Phusion Flash High Fidelity Master Mix (Thermo Fisher), the thermocycling parameters for PCR were 98° C. for 2 min, 35 cycles of (98° C. for 1 s, 60° C. for 5 s, 72° C. for 1 min) and 72° C. for 2 min. The DNA was purified and sequenced by Sanger sequencing.

### Results

Based on the strong persistence dynamics in both the time-course co-culture and cancer model scored by multiple independent crRNAs, the PRDM1 variant represented a promising candidate for CAR-T engineering. PRDM1 was previously identified as a master regulator of normal CD8 T cells (Rutishauser et al., Immunity, 31:296-308. (2009)). It was hypothesized that PRDM1 editing may have enhanced potential for promoting CAR-T cells' anti-tumor immunity. To further investigate whether targeting of PRDM1 can improve CAR-T cell persistence and other anti-tumor effects, a series of experiments were performed. First, the cutting efficiency of multiple PRDM1 crRNAs was measured individually in donor 2 anti-CD22 CAR-T cells (CD22 CARs) by T7E1 endonuclease assay and NGS. Most of the PRDM1 crRNAs exhibited high efficiency gene editing (6/8 over 80%; 7/8 over 78%; only 1/8 below 50%—PRDM1-cr7 at 47%). Next, PRDM1-cr1 cutting efficiency in another healthy donor was tested along with another form of PRDM1 mutant CAR-T cells (anti-CD19 CAR-T cells/CD19 CARs).

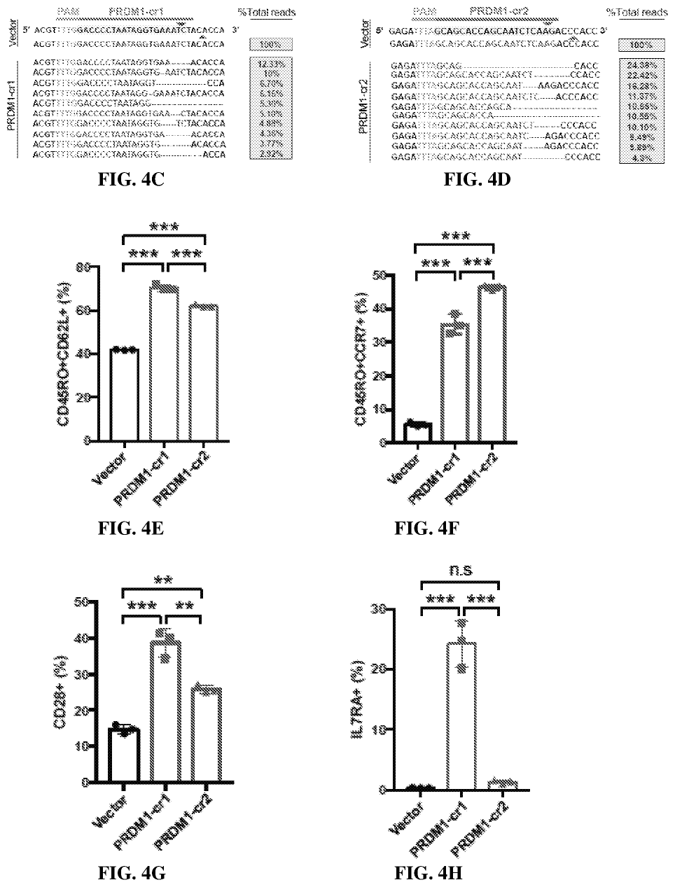

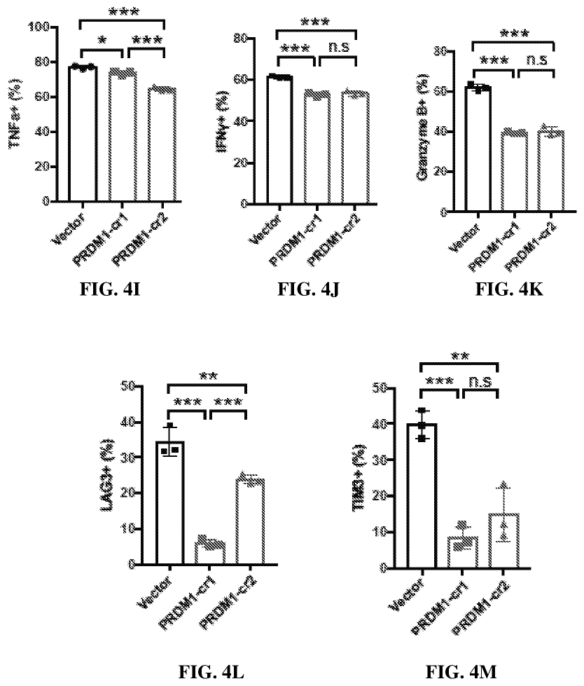

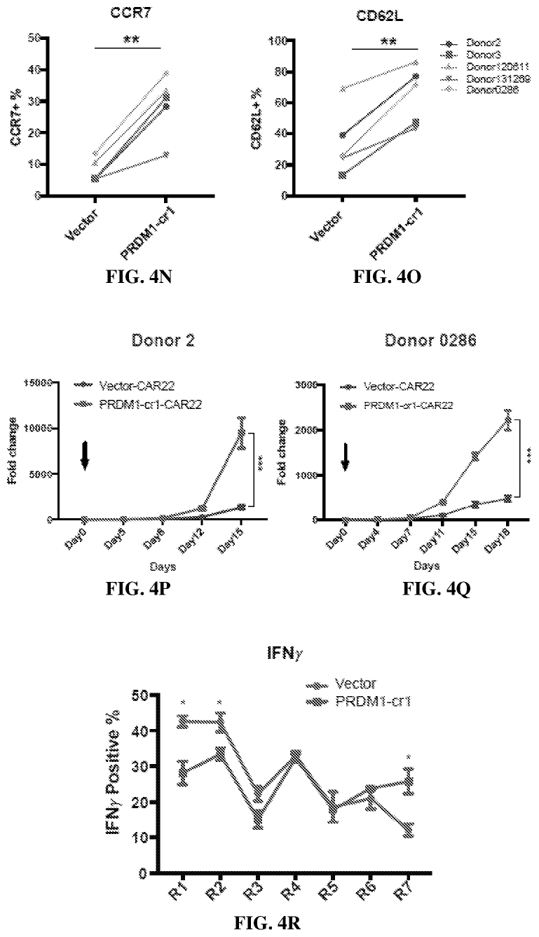

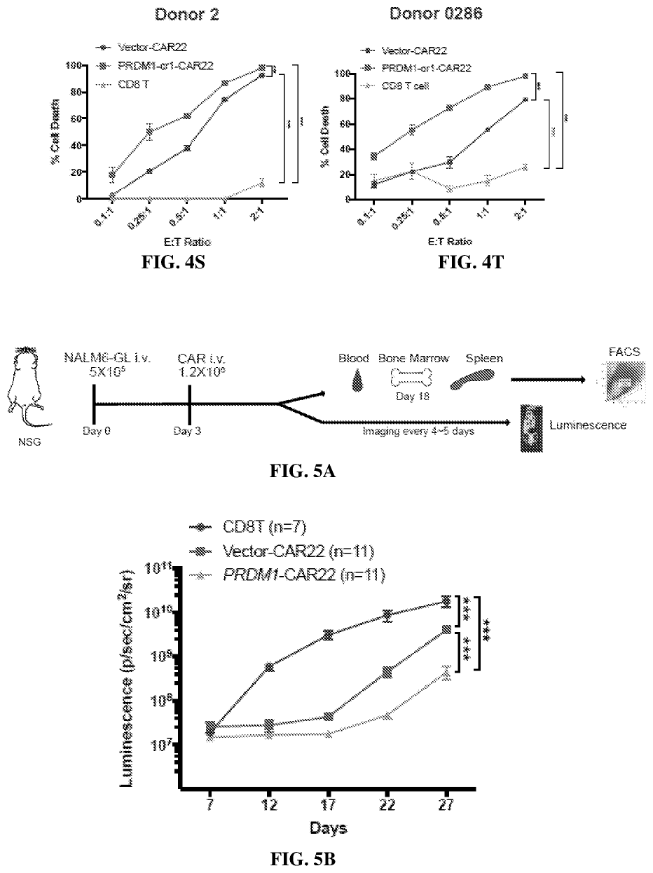

The top two crRNAs scoring at high abundance at late-stage time points in the co-culture dynamics were chosen to further examine how the PRDM1 crRNAs affect the functional gene products (mRNA and protein) in CAR-T cells. These two PRDM1 crRNAs target different domains of PRDM1 protein. PRDM1 protein has three different isoforms produced by alternative splicing (UniProtKB—O75626; FIG. 4A). To investigate whether these two PRDM1 crRNAs can disrupt PRDM1 mRNA expression, three different probes specific to the mRNA transcripts that potentially encodes these isoforms were designed (Probe ISO1 targeting 5 prime region of isoform1, ISO2 targeting PRDM1-cr1 cutting site within the SET/PR domain, and ISO3 targeting PRDM1-cr2 cutting site within the zinc finger domain of PRDM1). It was observed that disruption of PRDM1 gene led to sharp increase of PRDM1 mRNA transcript as detected by ISO1 probe. This phenomena is consistent with previous studies where PRDM1 is autoregulated by itself via a strong feedback mechanism (Magnúsdóttir E., et al., Proc Natl Acad Sci USA., 104(38):14988-93 (2007); Martins G., and Calame K. Annu Rev Immunol., 26:133-169 (2008)). However, the PRDM1 mRNA could not be detected by the ISO2 probe in PRDM1-cr1 CAR-T cells, or by the ISO3 probe in PRDM1-cr2 CAR-T cells.

To further investigate this mechanism, the PRDM1 protein expression was further tested in anti-CD22 CAR-T cells generated from different donors. Two different AAV-CLASH vectors targeting different domains of PRDM1 were generated, with the top two crRNAs scored at high abundance at late-stage time points in the co-culture dynamics. These CLASH vectors were used to transduce human primary CD8 T cells to generate two forms of PRDM1 mutant anti-CD22 CAR-T cells (PRDM1-cr1 and PRDM1-cr2), along with one form of anti-CD19 CAR-T cells (PRDM1-cr1 only) (FIG. 4A). Both crRNAs generated high-efficiency PRDM1 gene editing at the target sites when examined 5 days post-transduction on different donors (FIG. 4B-4D). To further examine how the PRDM1 crRNAs affect the functional gene products (protein and mRNA) in CAR-T cells, PRDM1 protein expression in CAR22 T cells generated from different donors was tested. It was observed that PRDM1-cr2 led to strong reduction of the PRDM1 protein, yet interestingly, PRDM1-cr1 led to a production of a smaller sized protein recognized by the same PRDM1-specific antibody. To investigate the sequence of this reduced-size protein, immunoprecipitation was performed using anti-PRDM1 antibody and peptide identification by mass spectrometry (IP-MS). IP-MS results showed that the peptides near the PRDM1-cr1 cutting sites cannot be detected in PRDM1-cr1 targeted PRDM1 mutant CAR-T cells, as compared with vector control CAR-T cells, in all three replicates. Thus, PRDM1-cr1 generated a new mutant variant that is neither isoform2 nor isoform3.

Because specific protein domains targeted by CRISPR mediated gene editing with different guide RNAs can result in different functional mutants, it is often important to reveal the specific mutations in functional domains. To determine the nature of the mutant variant of PRDM1 gene product produced by PRDM1-cr1, two primers near the PRDM1-cr1 cutting site were designed and RT-PCR was used to identify the cDNA. Interestingly, two bands presented in PRDM1-cr1 group. The top band represents a mixture of wildtype and similar size gene products with small indels (reflected by the “noise” peaks), consistent with the Nextera-NGS results. Moreover, there was a 120 bp in-frame deletion in the lower DNA fragment which precisely corresponded to PRDM1 exon3. Gene editing induced exon-skipping has been observed in Kras and Ctnnb1 in mouse cell lines using by CRISPR/Cas9 (Mou, H. et al., Genome Biol 18, 108, doi:10.1186/s13059-017-1237-8 (2017)). PRDM1 exon3 is the main region responsible for encoding the N-terminal PR domain in PRDM1 (PRDI-BF1 or Blimp-1) protein. Previous studies have shown that disruption of the PR domain can result in a dramatic loss of repressive function on multiple target genes. These results demonstrated that CLASH PRDM1-cr1 generated a PRDM1 exon3 skipping variant and produced a truncated PRDM1 protein in human primary T cells.

To compare PRDM1-cr1 and PRDM2-cr2 function on CD8⁺ T cells and CD22 CAR-T cells, cells generated 5 days post transduction were analyzed by flow cytometry, assessing phenotypes of effector-to-memory transition (CD62L, CCR7, CD28 and IL7R), cytotoxicity (IFNγ, TNFα, and Granzyme B/GZMB), and T cell exhaustion (LAG3 and TIM3). The data showed that both PRDM1-cr1 and PRDM2-cr2 variants have similar phenotypes, including higher effector-to-memory transition markers (FIGS. 4E-4H), lower effector markers as evident in GZMB, IFNγ and TNFα while human PBMC-derived CD8 T cells without CAR showed baseline effector cytokine levels across all groups (FIGS. 4I-4K), and lower exhaustion markers as evident in LAG3 and TIM3 (FIGS. 4L-4M). Interestingly, PRDM1-cr1 mutant CAR-Ts have more pronounced effects on CD62L, CD28, IL7R, TIM3 and LAG3 phenotypes as compared with PRDM1-cr2 (FIGS. 4E-4F, 4L, 4M).

To further confirm the PRDM1 effect on the CAR-T cells, the memory markers CCR7 and CD62L on different healthy donors were measured 5 days post transduction. Consistent with previous results, both markers were increased by editing of PRDM1-cr1 in all five donors (FIGS. 4N-4O). Additionally, PRDM1 mutant CAR-T cells were found to have significantly higher antigen-specific proliferative capacity and cytotoxicity than vector CAR-T cells in response to NALM6 cancer cell stimulation in two different donors, although the proliferation effect only become prominent after d11/d12 post transduction (FIGS. 4P-4Q). Long-term cytokine release also was monitored for each round of stimulation. It was observed that IFNγ production in PRDM1 CAR-T cells in response to specific antigen was lower than that of vector CAR-T cells at the beginning. However, while vector CAR-T cells continued to lose the ability to produce IFNγ each round, PRDM1 CAR-T cells were able to maintain the ability, and as a result, had a higher proportion of IFNγ producer cells than vector CAR-T cells at the endpoint of the experiment (Round 7) (FIG. 4R). Little difference was observed between the two groups for TNFα. Granzyme B was consistently lower in PRDM1 CAR-T cells as compared to vector. In the co-culture experiment endpoint, it was observed that as compared to vector cells, the long-term cultured, antigen-experienced PRDM1 CAR-T cells have significantly higher cytotoxic effect against NALM6 cancer cells, across all E:T ratios, in CARs generated from three independent donors (FIGS. 4S-4T). These data indicated that PRDM1 mutant CAR-Ts have enhanced memory phenotypes and are capable of maintaining longer-term effector function under continuous antigen exposure.

Genome-Wide Profiling of CLASH-PRDM1 Mediated AAV Integration

Previous studies have shown that the CRISPR/Cpf1 system has higher editing specificity compared to Cas9 nuclease by using GUIDE-seq, Digenome-seq and BLISS (Kleinstiver, B P. et al., Nature biotechnology 34, 869-874 (2016); Kim, D. et al., Nature biotechnology 34, 863-868 (2016); Yan, W X., et al., Nature communications 8, 1-9 (2017)). A recent study performed deep profiling and revealed the heterogeneity of integration outcomes in CRISPR knock-in experiments (Canaj, H. et al., bioRxiv 841098; doi: 10.1101/841098 (2019)). Considering these studies, in order to profile and quantify the CLASH-PRDM1 mediated genome-wide AAV integration, a new method based on GUIDE-seq (Tsai, S Q. et al., Nat Biotechnol., 33:187-197 (2015)) was developed and applied to CLASH-PRDM1 CAR-T cells with an AAV-only control without Cpf1 mRNA electroporation. The genomic DNA samples were collected in triplicate and sheared by a sonication, and the products were ligated with adaptors after end-repairing and dA-tailing. A 5′ biotinylated primer which targets a unique part of the CLASH vector was designed to bait the integrated sequences with unknown prey sequences. These junctions containing CLASH unique sequences were enriched via binding to streptavidin-coated magnetic beads. The sequencing barcodes were added during second round nested PCR.

Using ITR-based query sequences, a computational pipeline was established to identify chimeric off-target reads and their locations in the human genome. Integrative Genomics Viewer (IGV) based visualization of the normalized reads throughout the human genome showed a clean baseline level in the AAV-only control, and a small number of detectable peaks for CLASH-PRDM1 samples. Circos plot visualizations showed a similar pattern, with the distribution of off-target integration events throughout the human genome and relative frequencies, with the locations of peaks labelled at the center. It was observed that the mean genome-wide sum frequencies of off-target integration events in CLASH-PRDM1 samples was 0.62%, compared to 0.15% in samples receiving only the AAV vector without the Cpf1 mRNA. Certain detectable off-target integration events were observed at genomic loci around CD8A, TUBA1B, PVT1, TRAC, and PRDM1. For example, the mean off-target integration frequency at the PRDM1 locus was 0.2%, 0.05% at the TRAC locus, and 0.1% at the CD8A locus. Further zoom-in views of IGV-based visualizations showed the locations for the off-target integration events at the individual gene level. These experiments measured the genome-wide AAV off-target integration events occurring with CLASH-PRDM1 CAR-T generation, and estimated an overall genome-sum off-target integration rate at sub-percentage levels.

### Example 5: PRDM1 Mutant CAR-Ts Show Enhanced Therapeutic Efficacy In Vivo

### Materials and Methods

PRDM1 vs Control CD22 CAR-T Time-Course mRNA-Seq Experiment

T cells were infected with CLASH vector and PRDM1-cr1 CAR22 AAV6 after electroporation with NLS-LbCpf1mRNA. After electroporation for 5 days, the percentage of positive CAR-T cells was determined by staining with CD3 and CAR specific antibodies as previously described. CAR-T cells were co-cultured with NALM6 at low E:T (0.2:1) ratio every 4-7 days and a total of 5 rounds was performed. CAR-T cells were harvested using TRIzol (Invitrogen) after each round of stimulation. RNA was extracted using RNeasy Plus mini isolation kit (Qiagen). mRNA library preparations were performed using a NEBNext® Ultra™ RNA Library Prep Kit for Illumina and samples were multiplexed using barcoded primers provided by NEBNext® Multiplex Oligos for Illumina® (Index Primers Set 1). Libraries were sequenced with Novaseq systems (Illumina).

mRNA-Seq Processing

FASTQ files from mRNA sequencing were analyzed using the Kallisto quant algorithm with setting-b 100 for transcript quantification (Bray N L., et al., Nature biotechnology, 34:525-527 (2016)). Differential expression analysis was performed using Sleuth (Pimental H., et al., Nat Methods, 14(7):687-690 (2017)). Differentially upregulated and downregulated genes were selected for DAVID analysis using a Q value cutoff of 1×10⁻³ (Sherman B T., and Lempicki R A., Nat. Protoc., 4(1):44-57 (2009)). Z-scores for time course heatmap were calculated by log 2-normalizion of gene counts following by scaling by genes, and differentially expressed genes across the time points were determined by limma with contrasts set up to compare PRDM1 vs vector control CAR-T cells at each time point. Time course cluster analysis was performed with the R package maSigPro using “two.ways.forward” as step method and otherwise default settings. Visualizations of differentially expressed genes such as volcano plots and heatmaps were generated using standard R packages such as ggplot2 and VennDiagram.

In Vivo CAR-T Efficacy Testing in Mouse Models

NOD.Cg-Prkdc^{scid} Il2rg^{tm1Wj1}/SzJ (NSG) mice were purchased from the Jackson Laboratory and bred in-house. Both male and female NSG mice between 6-8 weeks old were used and inoculated with 5×10⁵ NALM6-GL cells intravenously. Mice were randomly assigned to different groups prior to treatment. 1.2×10⁶ normal CD8 T cells, vector CAR-T cells or PRDM1 mutant CAR-T cells were infused into mice after 3 days. Bioluminescent signal for each mouse was captured using In Vivo Imaging System (IVIS) every 3-5 days. Briefly, to generate bioluminescence signals, XenoLight D-Luciferin (Perkin Elmer) was intraperitoneally injected into mice at 150 mg/kg and images were analyzed by living image software. To test T cell phenotypes in vivo after treatment, NSG mice was euthanized at day 18. Peripheral blood, spleen and bone marrow was collected immediately. Red blood cells were lysed by incubation with ACK (Ammonium-Chloride-Potassium) Lysing Buffer (Thermo Fisher) for 2 minutes. After wash, the cell surface markers were labeled as described above for FACS and measured by BD FACSAria II.

Randomization

In animal experiments, mice were randomized by sex, cage and littermates. In vitro experiments were not randomized.

Blinding

Investigators were blinded to the identity and treatment groups of animals when measuring tumor burden. Investigators were not blinded in in vitro experiments. In NGS data analysis, investigators were blinded for initial processing of the original data using key-coded metadata.

### Results

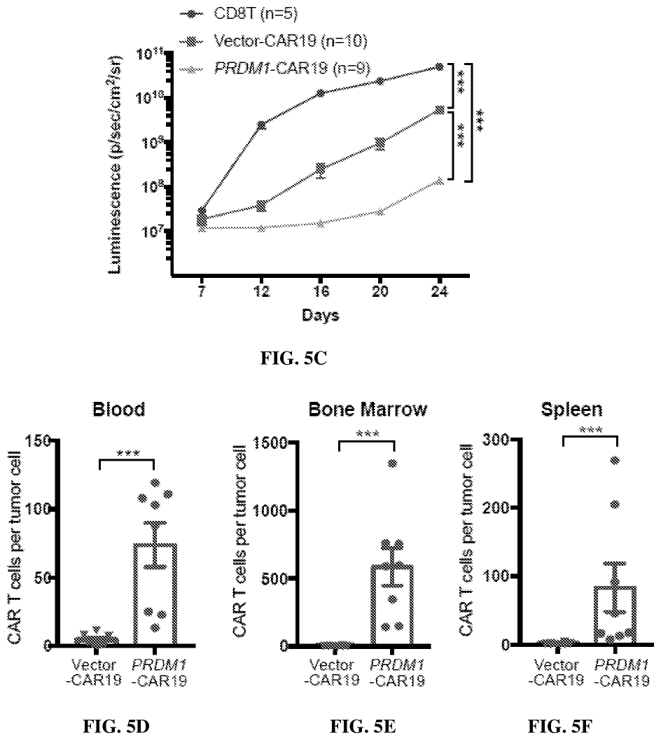

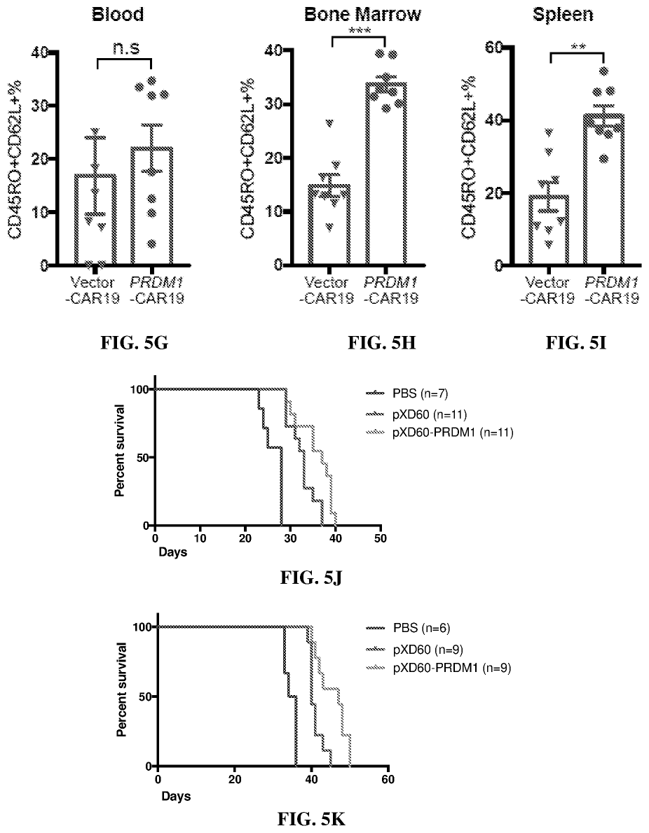

The pre-clinical therapeutic efficacy of PRDM1 mutant CAR-T cells compared to their vector counterpart was then tested in vivo. In a NALM6-GL leukemia tumor model in immunodeficient NOD.Prkdc^{(SCID)}/Il2rγ^{-/-} (NSG) mice, efficacy testing was performed by adoptive transfer of anti-CD22 CAR-Ts followed by monitoring of tumor burden (FIG. 5A). The NALM6 cells were first confirmed to be CD19; CD22 double positive prior to being used for tumor induction. IVIS imaging was performed to follow the tumor burden in leukemic animals treated by untransduced CD8 T cells, vector transduced anti-CD22 CAR-T cells, and PRDM1 edited anti-CD22 CAR-T cells and observed that PRDM1 mutant anti-CD22 CAR-T cells showed significantly enhanced leukemia suppression than vector CAR-T cells. Substantial differences were observed in tumor burden between vector and PRDM1 anti-CD22 CAR-T cell treated mice, starting 19 days post T cell adoptive transfer (22 days post tumor induction) (FIG. 5B). These data showed that PRDM1 mutant CD22 CAR-Ts have stronger in vivo efficacy against cancer in a mouse model of leukemia.

To further validate the results, an independent AAV-CLASH vector with a different CAR construct against the CD19 antigen (anti-CD19 CAR-T, CD19 CAR, CAR19) was constructed. PRDM1 mutant CD19 CAR-T cells were similarly generated along with vector control CD19 CAR-T cells, followed by in vivo efficacy testing using a similar cancer induction and adoptive transfer treatment regimen. Similar results were observed in CD19 CAR-T cells in vivo, where PRDM1 CD19 CAR-T cells showed significantly enhanced leukemia suppression compared to vector CAR-T cells (FIG. 5C). Blood and organs from the animals treated in both groups were isolated and the abundance of persistent CAR-Ts in vivo was quantified. Two weeks after CAR-T infusion, higher numbers of CAR-T cells and CAR-T to tumor ratio were observed in the blood, bone marrow and spleen of recipients of PRDM1 CAR-T cells (FIGS. 5D-5F). Consistent with in vitro findings, CAR-T cells from the bone marrow and spleen showed significantly higher levels of CD45RO⁺CD62L⁺ (Tcm) in mice treated with PRDM1 CAR-T cells as compared to vector group (FIGS. 5G-5I). These data indicated that PRDM1 mutant CAR-T cells have enhanced efficacy, along with increased persistence and memory marker expression in vivo.

The pre-clinical therapeutic efficacy of PRDM1 mutant CAR-T cells (compared to vector counterpart) was further evaluated in vivo, in the context of survival of cancer-bearing animals. A NALM6-GL leukemia tumor model in immunodeficient NOD.Prkdc(SCID)/Il2rγ−/− (NSG) mice, was used to perform efficacy testing by adoptive transfer of anti-CD22 CAR-Ts followed by assessment of end-point survival. End-point survival was recorded either as poor body condition score (BCS <=1), or actual death, whichever was earlier. Two experiments were performed, one with adoptive transfer of CAR-T cells at day 3 post tumor induction, the other with adoptive transfer at day 8. In both experiments, it was observed that leukemia-bearing animals treated with PRDM1 mutant CD22 CAR-T cells showed significantly longer survival than animals treated with vector CAR-T cells (FIGS. 5J-5K). These data show that PRDM1 mutant CD22 CAR-Ts have stronger in vivo efficacy against cancer in a mouse model of leukemia.

To further validate these results, PRDM1 mutant CD19 CAR-T cells were similarly tested along with vector control anti-CD19 CAR-T cells. The in vivo efficacy testing was performed as above for anti-CD22 CAR, but with adoptive transfer of CAR-T cells only at day 3 post tumor induction. Results showed that leukemia-bearing animals treated with PRDM1 mutant anti-CD19 CAR-T cells showed significantly longer survival than animals treated with vector CAR-T cells (FIG. 5L). These data show that PRDM1 mutant anti-CD19 CAR-Ts have stronger in vivo efficacy against cancer in a mouse model of leukemia.

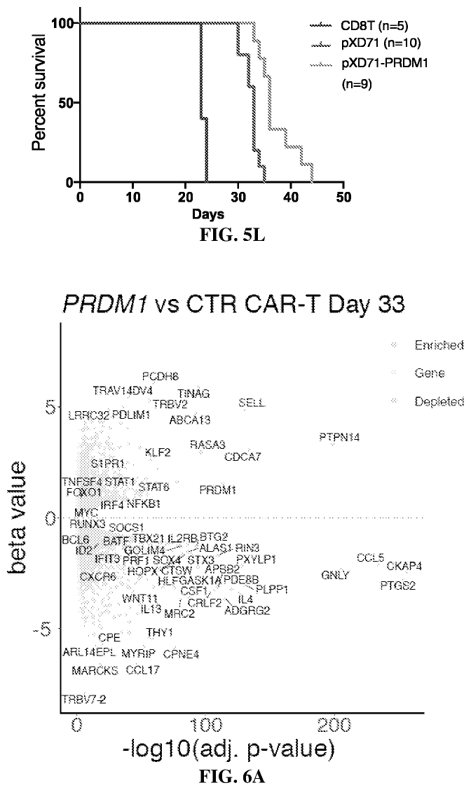

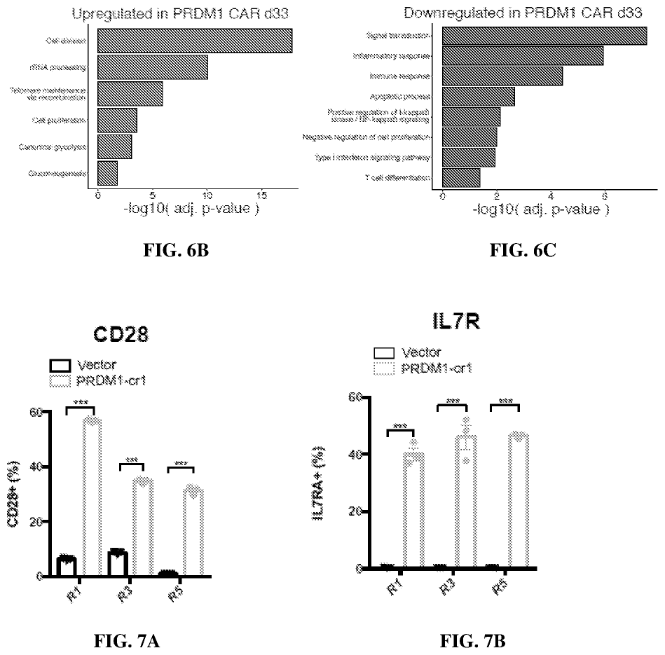

To further understand the molecular underpinnings of PRDM1 phenotypes and the basis for enhanced efficacy specifically in CAR-T cells, a time-course transcriptomics experiment was performed in biological triplicate in PRDM1 CAR-T cells, vector control CAR-T cells, and the untransduced human CD8 T cell as a baseline. These transcriptome profiles revealed a systematic landscape of CAR-T gene expression with continuous cognate cancer antigen stimulation along the time line. Distinct sets of differentially expressed genes were identified in the direct comparison between PRDM1 and vector control CAR-T cells (FIG. 6A), revealing a panel of highly significant downstream targets. PRDM1 CAR-Ts showed significant induction of genes such as PCDH8, SELL/CD62L, PTPN14, RASA3, KLF2, STAT6, STAT1, PRDM1 itself, IRF4 and NFKB1; and repression of genes such as CCLS, BATF, CXCR6, IL13, PRF1, IFIT13, ID2 and RUNX3 (FIG. 6A).

Interestingly, the alterations in gene expression showed a gradient pattern in both directions; increasing expression of PRDM1-induced genes and decreasing expression of PRDM1-repressed genes with time (thus along additional rounds of stimulation). To rigorously quantify and deconvolve the time factor effect, a time-series cluster analysis was performed. MaSigPro time course cluster analysis revealed experiment-wide expression profiles of different gene clusters each containing genes with similar expression patterns along time and similar behaviors between PRDM1 mutant vs control CAR-T groups. This analysis revealed nine distinct clusters of genes with completely different behaviors along the time course between the two groups: Cluster 1 genes decrease over time (thus stimulation) for both PRDM1 and vector control CAR-T cells; Cluster 2, the largest cluster among all, contains genes that strongly increase over time in control CAR-T cells, but fail to do so in PRDM1 CAR-T cells (representative examples include HAVCR2 and WNT11); Cluster 3 contains genes that decrease over time in control, but in contrast behave the opposite, increasing in PRDM1 CAR-T cells (representative examples include STAT1, STAT6, NFKB1, and IRF4); Cluster 4 contain genes stable over time in control but which sharply drop in PRDM1 CAR-T cells; Cluster 5 contains genes stable over time in control but sharply increase in PRDM1 CAR-T cells; Cluster 6 contains genes that sharply decrease over time in control but remain stable in PRDM1 CAR-T cells (representative examples include KLF2, FOXO1, CD28, CD226, CDCA7); Cluster 7 are also dichotomy genes where the two groups are heading to opposite directions over time, where genes increase in control but decrease in PRDM1 group; Cluster 8 contains genes that increase over time in control, however they first rapidly drop and then become stable at low levels in the PRDM1 group (representative examples include LAG3 and ID2); the final and also the smallest cluster, Cluster 9, contains genes with shared and consistent induction patterns in both PRDM1 and vector control CAR-T cells.

To gain an overall picture of what pathways are enriched in the different clusters of genes, Gene Ontology analysis of enriched biological processes of the gene sets in each cluster was performed. The two clusters with similar gene set behaviors (Clusters 1 and 9) are both enriched in transcription factors. Interestingly, the clusters where PRDM1 and vector control CAR-T cells behave differently have distinct signatures: genes in Cluster 2 where PRDM1 editing inhibits the time-dependent inductions are enriched in signal transduction, cell adhesion and inflammatory responses; similarly, genes in Cluster 4 where PRDM1 editing led to rapid downregulation are enriched in chemokine-mediated signaling pathway, positive regulation of inflammatory response, negative regulation of type I interferon production and immune response. Concordantly, in Clusters 7 and 8 where genes in PRDM1 CAR-T are suppressed rather than induced, enriched pathways include negative regulation of T cell receptor signaling pathway, regulation of T cell activation and again, inflammatory response. In contrast, in Cluster 6 where PRDM1 prevents decrease of gene expression over time, strong signatures were found in proliferation, including mitotic nuclear division, cell division, chromosome segregation, sister chromatid cohesion, cell proliferation, GUS transition of mitotic cell cycle and DNA replication, consistent with the phenotype that PRDM1 CAR-T cells maintain the strong proliferative capability even after continuous antigen stimulation and multiple rounds of cancer cell killing. Differential expression analysis was also performed on the same dataset using pairwise group comparisons. Although by nature, non-clustering based differential expression analysis cannot capture cluster-specific signatures, these differential expression results from the three time points also validated the collective signatures of T cell proliferation and apoptosis, T cell differentiation, signal transduction, inflammatory response and immune responses in PRDM1 CAR-T cells (FIGS. 6B-6C).

### Example 6: PRDM1 Mutant CAR-Ts Rewired Multiple Immunological Programs

### Materials and Methods

RT-PCR

RNA was extracted as described in RNA-seq. cDNA for qPCR was generated using M-MLV reverse transcriptase enzyme (Sigma) and Oligo dT (Thermo Fisher) following the manufacture's protocol. For the PRDM1 mutant sequence analysis, PCR was performed by using primers near the PRDM1-cr1 cutting site by using the cDNA as template. For the RNA-seq validation, the cDNA was subjected to qPCR using TaqMan Real-Time PCR Master Mixes and Taqman gene assay probes (Thermo Fisher). Samples were processed using Applied Bioscience Step One Plus Real Time machine and relative mRNA expression was normalized to GAPDH controls. Relative mRNA expression was determined via the AA C method.

### Results

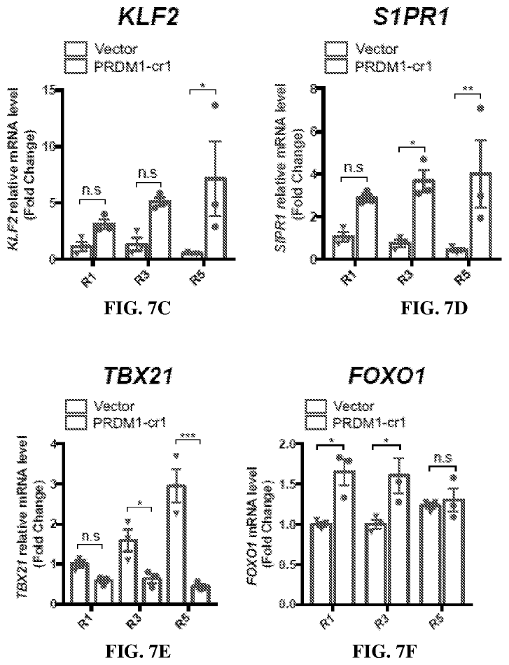

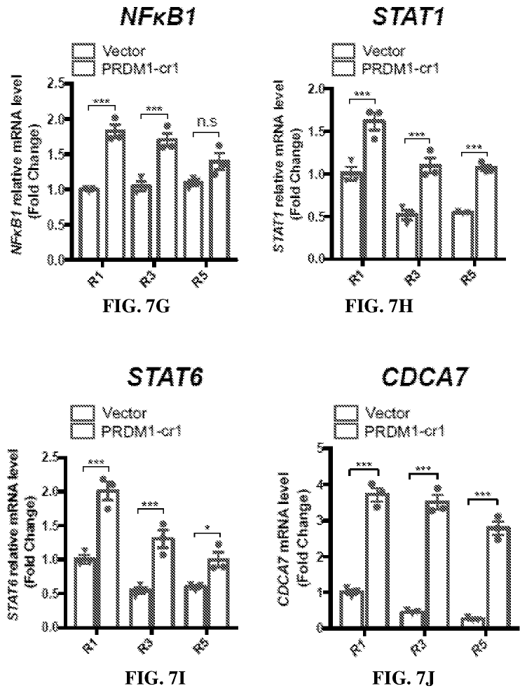

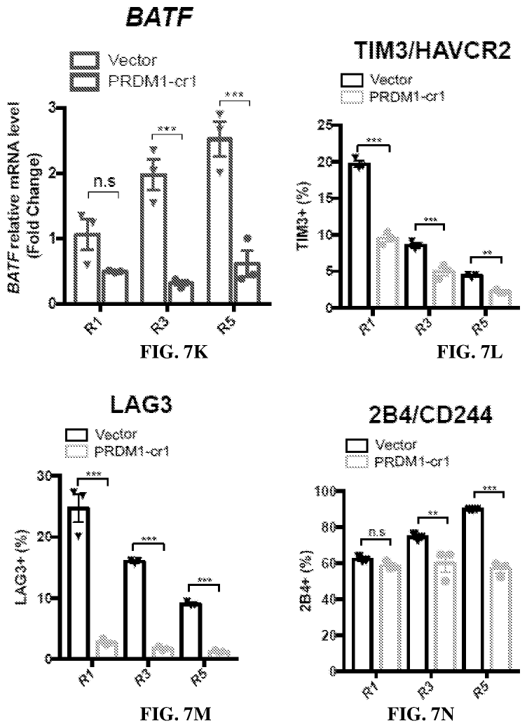

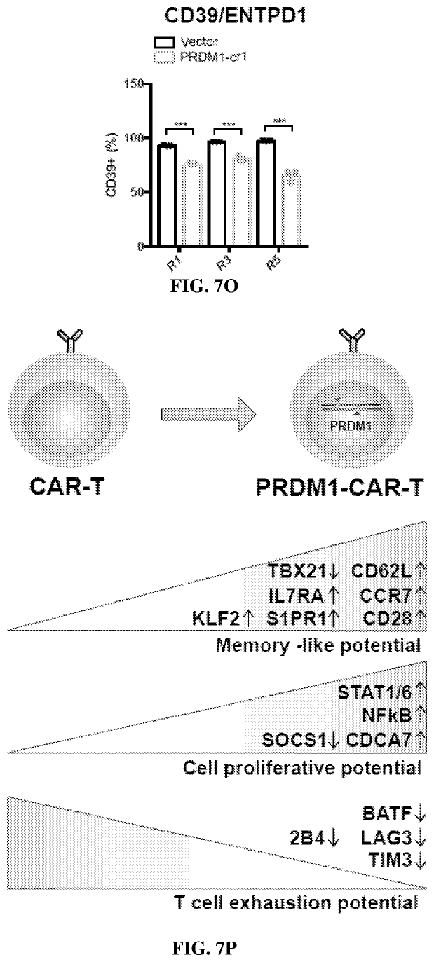

The gene expression signatures in PRDM1 edited CAR-T prompted investigation into the underlying immunological programs, such as T cell differentiation, memory features, T cell exhaustion, T cell activation, cytokine and chemokine production, as well as signaling pathways. It was observed that some Cluster 6 genes, CD28 and IL7R, two of the T cell memory surface markers, were significantly upregulated across all 3 time points (rounds of NALM6 stimulation) in PRDM1 edited CAR-T as compared to vector control (FIGS. 7A-7B). Consistent with the time-course RNA-seq, the upstream regulators such as KLF2 and S1PR1, also Cluster 6 genes, exhibited a descending trend with continuous antigen exposure, but this effect was reversed by editing of PRDM1 in CAR-T cells (FIGS. 7C-7D). Transcription factors (TFs) that regulate the differentiation of effector and memory T cells were then examined (Chang J T., et al., Nat Immunol., 15(12):1104-15 (2014); Michelini R H., et al., J Exp Med. 210(6):1189-200 (2013)). It was observed that effector-driving TFs such as TBX21, ID2 and RUNX3, were significantly downregulated in PRDM1 mutant CAR-T cells. In contrast, FOXO1, a factor essential for the formation of long-lived memory cells, was upregulated in PRDM1 CAR-T cells in the first two rounds of stimulation (FIGS. 7E-7F). In addition, NFKB1, STAT1, STAT6, and CDCA7, master regulators for the proliferation of T and other immune cells associated genes, were significantly increased in PRDM1 CAR-T cells as compared to vector control cells, and gradually diminished over time with continuous antigen stimulation (FIGS. 7G-7J). On the other hand, IFIT3 and SOCS1, genes that inhibit proliferation of T cells, were downregulated in a time-dependent manner in PRDM1 CAR-T cells as compared to vector control. The alterations of these underlying major pathways are consistent with the increased persistence and sustained proliferative capabilities of PRDM1 CAR-Ts.

Other pivotal markers for pathways of T cell function were further examined in PRDM1 CAR-T cells. BATF, a Cluster 7 gene that encodes an AP-1/ATF superfamily of transcription factor that regulates effector CD8 T-cell differentiation (Kurachi M., et al., Nat. Immunol., 15(4):373-83 (2014)), was rapidly induced with antigen stimulation in regular vector control CAR-T cells, but this induction was abolished in PRDM1 mutant CAR-T cells (FIG. 7J), consistent with the time-course RNA-seq. Several genes encoding inflammatory chemokines/cytokines including CXCR6 (Cluster 7), IL13 (Cluster 7) and PRF1 (Cluster 8) were also observed to be dramatically downregulated in PRDM1 mutant CAR-T cells as compared to vector control, again in concordance with the time-course RNA-seq. PTPN14, a Cluster 5 gene that encodes a member of the protein tyrosine phosphatase (PTP) family that regulates a variety of cellular processes including cell growth, differentiation, mitotic cycle, and oncogenic transformation (Pike K A. and Tremblay M L., Front. Immunol., 9:2504 (2018)), was highly induced in PRDM1 CAR-T cells as compared to vector control. In contrast, WNT11, a Cluster 2 gene that encodes a WNT/beta-catenin pathway signaling receptor (Van Loosdregt, J., and Coffer, P J., J. Immunol., 201(8):2193-2200 (2018)), was rapidly induced by NALM6 stimulation in vector control T cells, but its induction was completely abolished in PRDM1 CAR-T cells, a signature of the Cluster 2 gene behavior. In addition, PCDH8 showed strong induction in PRDM1 CAR-T cells, but not vector control CAR-T cells, and RIN3 showed strong induction in vector control but not PRDM1 CAR-T cells.

It was hypothesized that perturbation of PRDM1 may reduce T cell exhaustion in CAR-Ts. Consistent with the time-course RNA-seq, flow cytometry analysis showed that PRDM1 CAR-T cells have decreased levels of TIM3, a canonical immune checkpoint encoded by the HAVCR2 gene that also falls into Cluster 2 of the RNA-seq time course (FIG. 7L). Additional surface checkpoints such as LAG3, 2B4/CD244 and CD39/ENTPD1 were also significantly and consistently reduced on the T cell surface (FIGS. 7M-70), indicating a robust dampening of exhaustion in PRDM1 CAR-T cells. Together, these data demonstrate that PRDM1 CAR-T cells showed improvement over control CAR-Ts via increased memory phenotype, decreased T cell terminal differentiation, enhanced cell proliferation, and reduced T cell exhaustion upon chronic cancer antigen exposure (FIG. 7P).

CLASH is a versatile platform for massive-scale engineering of CAR-T cells, which is currently viewed as “living drugs” in immunotherapies. In contrast to non-viral and DNA-based cDNA transgene knock-ins, the CLASH system takes advantage of AAV vectors which allow for highly efficient human T cell transduction as well as large scale perturbations, by simply creating the viral vectors in a pooled manner. CRISPR/Cas9 gene editing for targeted delivery of a CAR gene into a specific locus such as TRAC, can enhance T cells potency and increase tumor rejection compared to random integration in retroviral or lentiviral vectors (Eyquem J., et al., Nature 543, 113-117 (2017)). Non-viral DNA electroporation has been used to create a medium number (36) of transgenes knocking into the genome of normal human T cells (Roth T L., et al., Cell, 181(3):728-744.e21 (2020)), but not CAR-T cells.

The CLASH system facilitated AAV-HDR mediated CAR-T knock-in into TRAC and introduction of a third perturbation by carrying another user-defined crRNA in the same vector. The flexibility of the wildcard crRNA as well as the simplicity to readily scale up large numbers of crRNAs in a pool makes it simple to perform massively parallel perturbations by CLASH, in contrast to the limitations of cDNAs that differ between each construct and are difficult to scale up high. Thus, CLASH allows for simultaneous transduction of large numbers of human T cells to engineer stably knocked-in CAR constructs with massively targeted diversity. The resulting pool of T cell variants therefore immediately permit high-throughput selection or screening of desired phenotypes from the pool in an unbiased and quantitative manner. As demonstrated in the examples, the representation of the knock-in pool can be directly read out by next generation sequencing (NGS).

Large-scale CRISPR screens have been applied to human and mouse primary T cells with lentiviral vectors (Dong M B., et al., Cell, 178(5):1189-1204.e23 (2019); Shifrut M., et al., Cell, 175:1958-1971. e1915 (2018); Ting P Y., et al., Nat. Methods, 15(11):941-946 (2018); Ye L., et al., Nat Biotechnol., 37(11):1302-1313 (2019)), and more recently by the transposon system. However, unlike existing lentiviral vector or transposon based CRISPR libraries that randomly integrate into the genome, the CLASH system precisely targets all CAR-T variants into the same locus, thus creating a series of variants controlling for position effect and thereby avoiding insertional mutagenesis in the genome of CAR-T cells. The tracr-independent Cas12a/Cpf1 system also facilitates multiplexed targeted mutagenesis as the same polymerase III promoter can drive a string of crRNAs (e.g., crTRAC-crWildcard), to allow the whole knock-in/knock-out construct to comfortably fit into the 4.7 kb packaging limit of an AAV vector. Besides T cells, the CLASH technology can be applied to many other cell types, such as, other primary immune cells or stem cells.

The CLASH system was used to comprehensively interrogate immunologically relevant genetic perturbations that enhance CAR-T cell persistence upon long-term cancer antigen stimulation. To maximize the probability of hitting clinically relevant targets, immune-focused, T cell-centric libraries, Rene and Descartes were designed. The AAV-CLASH-Descartes library efficiently generated a large, functionally diverse CD22 CAR-T cell pool, which was subjected to long-term CAR-T culture with antigen specific cancer cell co-culture. The co-culture system itself exhibited a phenotype consistent with exhausted CAR-T cells, which was accompanied by increased T cells terminal differentiation, low proliferation and poor cytokine release capacity (Wherry E J., Nature immunology 12, 492-499 (2011)). The long-term co-culture selection pressure enriched for a series of genes which could promote CAR-T cell survival by increasing killing ability, cell proliferation or overcoming T cell exhaustion. Among these genes, TE72, one of the top hits, has been shown to improve the efficacy and persistence of CAR-T cells after disruption (Fraietta et al., 2018), demonstrating the validity of the platform.

Systematic deconvolution of the CLASH CAR-T time-course library dynamics using NGS and analysis, revealed a comprehensive map of the quantitative effects of individual genes in the Descartes library. This identified a number of candidates that modulate the function in CAR-T cells. PRDM1 CAR-T cells displayed a central memory phenotype which mediated potent anti-tumor effects in advanced leukemia. Finally, time-course RNA-seq analysis permitted a dynamic assessment of the functional consequences of perturbing PRDM1.

PRDM1 was previously known as a critical transcriptional regulator for B cell and T cell differentiation (Rutishauser et al., 2009). Recent studies uncovered that PRDM1 mediated T cell exhaustion via upregulation of TIGIT and PD-1 in patients (Zhu L., et al., J Hematol Oncol., 10(1):124 (2017)). However, these were done in normal primary cells but not CAR-Ts. Thus, this interrogation was performed directly in the CAR-T setting. With the interesting case of PRDM1-cr1, this construct is capable of generating a unique exon3-skip mutant of PRDM1 resulting in a truncated protein product. With CLASH engineered CAR-Ts, it was observed that PRDM1 perturbation in CAR-T cells manifest increased proliferative capacity and central memory phenotype in vitro and in vivo in both CD22 CAR and CD19 CAR settings. PRDM1 CD22 CAR and CD19 CAR both showed superior efficacy compared to the respective control CARs. The examples demonstrate that PRDM1 CAR-Ts are better than the control counterparts in leukemia models.

PRDM1 has been previously reported to recruit proteins or corepressor complexes to modify histones and repress transcription such as G9a histone methyl transferases (Gyory I., et al., Nature immunology 5:299-308 (2004)). Additional research has found a number of differentially expressed genes containing predicted PRDM1 binding sites, such as BCL6, ID3 and cMYC (Crotty S., et al., Nature immunology 11, 114-120 (2010); Martins G. and Calame K. Annu Rev Immunol., 26:133-169 (2008)). Knockout of PRDM1 in CD8 T cells sustained cytokine responsiveness and increased proliferation resulted from increased expression of CD25 and CD27 (Shin H M., et al., Immunity 39, 661-675 (2013)). The above studies on the mechanisms of PRDM1 perturbation in CAR-T cells showed certain conserved pathways, however these were not identical to previous studies potentially due to the CAR-T specific setting. BCL6, CD25 and CD27 were not increased after editing of PRDM1 in CAR-T cells. It is possible that the PRDM1-targeting crRNA did only change the PR domain and therefore leave the zinc finger domain unaltered. Previous studies reported promoter hypermethylation-mediated silencing of PRDM1 may contribute to the pathogenesis of Natural killer/T-cell lymphoma (NKTCL) (Iqbal J., et al., Leukemia 23, 1139-1151 (2009)). However, the loss-of-function mutations of PRDM1 are rarely observed in NKTCL (Kugiik C., et al., Ther Adv Med Oncol. 12:1758835919900856 (2020)). Given the fact that no pathogenic or oncogenic transformation of PRDM1 engineered CAR-T cells was observed, and the fact that most of CAR-T cells fade away in vivo due to lack of persistence, rather than over-proliferation, it is plausible that PRDM1 editing can confer a favorable therapeutic window to allow enhancement of CAR-T therapeutic efficacy with a manageable toxicity or other risks of side effects.

Though the Examples demonstrate the CAR-T CLASH system in leukemia models, this system can also be applied to other tumor types and other forms of CAR-Ts, simply by switching the knock-in CAR construct. There are challenges to successfully applying CAR-T cells to solid tumors due to poor T cell trafficking ability and the immunosuppressive environment in many advanced solid tumors (Lim, W A., and June, CH., Cell 168:724-740 (2017)). These challenges can be addressed using CAR-T cell tumor infiltration or time-dependent persistence models in vivo, and selecting new variants of CAR-Ts to enhance solid tumor CAR-T persistence, or, in certain more sophisticated settings, utilizing multiple transgenic reporters or immune markers for massively parallel engineering of new CAR-Ts to overcome suppressive components in the tumor microenvironment. To overcome various immune defects associated with cancer treatment and simplify the manufacturing of CAR-T cells, application of CLASH in models of allogeneic or universal CAR-T systems can facilitate the development of new strategies, expanding the current methods such as using gene editing to remove functional endogenous TCR. The versatility of the CLASH system opens multiple new venues for on-demand engineering of CAR-Ts with efficiency and precision.

Unless defined otherwise, all technical and scientific terms used have the same meanings as commonly understood by one of skill in the art to which the disclosed invention belongs. Cited publications and the materials for which they are cited are specifically incorporated by reference.

Those skilled in the art will recognize, or be able to ascertain using no more than routine experimentation, many equivalents to the specific embodiments of the described method and compositions. Such equivalents are intended to be encompassed by the following claims.

We claim:

1. A library comprising a plurality of two or more vectors, each vector comprising:
one or more inverted terminal repeat (ITR) sequences, a 5′ homology arm, a crRNA expression cassette, a chimeric antigen receptor (CAR) expression cassette, and a 3′ homology arm.

2. The library of claim 1, wherein the crRNA expression cassette of each vector independently encodes a first guide RNA and a second guide RNA, wherein the first guide RNA is identical across the plurality of vectors.

3. The library of claims 1 or 2, wherein the second guide RNA is unique to each vector across the plurality of vectors.

4. The library of any one of claims 1-3, wherein one or more sequences encoding the one or more of the encoded guide RNAs of the library are selected from the group consisting of SEQ ID NOs:3-12,134.

5. The library of any one of claims 1-4, wherein the library collectively comprises from about 100 to about 300,000, from about 1,000 to about 5,000 or from about 5000, to about 10,000 distinct guide RNAs.

6. The library of any one of claims 1-7, wherein the library collectively comprises guide RNAs encoded by SEQ ID NOs:3-4,087 (Rene library), SEQ ID NOs:4,088-12,134 (Descartes library), or SEQ ID NOs:3-12,134.

7. The library of any one of claims 1-6, wherein each crRNA expression cassette comprises a U6 promoter operably linked to sequences encoding one or more guide RNAs.

8. The library of any one of claims 1-7, wherein each crRNA expression cassette comprises sequences encoding a first guide RNA and a second guide RNA.

9. The library of any one of claims 1-8, wherein the CAR expression cassette comprises an EFS promoter and/or a polyadenylation signal sequence operationally linked to a sequence encoding the CAR.

10. The library of any one of claims 1-9, wherein the crRNA expression cassette and/or CAR expression cassette of each vector are positioned between the 5′ and 3′ homology arms.

11. The library of any one of claims 1-10, wherein the 5′ and 3′ homology arms are homologous to the TRAC locus.

12. The library of any one of claims 1-11, wherein each vector encodes at least one guide RNA targeting the TRAC locus.

13. The library of any one of claims 1-12, wherein the CAR targets one or more cancer specific antigens or cancer associated antigens.

14. The library of any one of claims 1-13, wherein the CAR is an anti-CD19 CAR or anti-CD22 CAR.

15. The library of any one of claims 2-14, wherein the second guide RNA targets a gene involved in T cell exhaustion, T cell proliferation, T cell co-stimulation, memory T cell differentiation, T cell receptor signaling, epigenetic regulation, adaptive immune response, immune response to tumor cells, other immune functions, or combinations thereof.

16. The library of any one of claims 1-15, wherein the vector
comprises the nucleotide sequence of SEQ ID NO:1 or SEQ ID NO:2 with or without the sequence encoding the TRAC targeting crRNA, with or without one or more additional crRNA encoding sequences optionally inserted at the BbsI cloning site, and/or with the existing CAR encoding sequence or another CAR encoding sequence substituted therefore,
or a sequence variant having 75% or more sequence identity to any of the foregoing.

17. The library of any one of claims 1-16, wherein each vector is a viral vector, preferably an adeno-associated virus (AAV) vector, optionally wherein the AAV is AAV6.

18. A vector of any one of claims 1-17.

19. A population of cells comprising an AAV vector of claim 18.

20. A population of cells collectively comprising the library of any one of claims 1-17, optionally wherein each cell comprises at most one or two AAV vectors comprised in the library.

21. A method of identifying one or more genes that enhance a desired phenotype of a cell comprising a CAR, the method comprising:
(a) contacting the population of cells of claim 20 with an RNA-guided endonuclease under conditions suitable for genomic integration and expression of the guide RNAs and CAR contained in the vectors; and
(b) selecting for cells exhibiting the desired phenotype.

22. The method of claim 21, wherein the crRNA expression cassette and CAR expression cassette are integrated into the TRAC locus.

23. The method of claim 21 or 22, wherein the RNA-guided endonuclease is provided as an mRNA that encodes the RNA-guided endonuclease, a viral vector that encodes the RNA-guided endonuclease, or an RNA-guided endonuclease protein or a complex of the RNA-guided endonuclease protein and RNA.

24. The method of claim 23, wherein the RNA-guided endonuclease is provided by electroporation.

25. The method of any one of claims 21-24, wherein the RNA-guided endonuclease is Cpf1 or an active variant, derivative, or fragment thereof.

26. The method of any one of claims 21-25, wherein the desired phenotype is selected from the group comprising increased tumor/tumor microenvironment infiltration, increased or optimized target cell affinity, increased target cell cytotoxicity, increased persistence, increased expansion/proliferation, reduced exhaustion, increased anti-cancer metabolic function, increased ability to prevent immune escape, reduced unspecific cytokine production, reduced off-target toxicity, reduced cytokine release syndrome (CRS), and combinations thereof.

27. The method of any one of claims 21-26, wherein the step of selecting comprises co-culturing the population of cells with target cells comprising one or more antigens recognized by the CAR for a defined time period, flow cytometry-based or affinity-based sorting, immune marker-based selection, in vivo tumor infiltration, CAR-antigen interaction, directed evolution, or combinations thereof.

28. The method of claim 27, wherein the population of cells is repeatedly co-cultured with the target cells.

29. The method of claim 27 or 28, wherein the time period comprises from about 1 to about 60 days.

30. The method of any one of claims 27-29, wherein the target cells comprise cancer cells.

31. The method of any one of claims 21-30, further comprising identifying the crRNA expression cassette present in the selected cells.

32. The method of claim 31, wherein the step of identifying the crRNA expression cassette comprises sequencing genomic DNA of the selected cells.

33. The method of claim 31 or 32, wherein the one or more genes that enhance a desired phenotype are identified as genes targeted by the guide RNAs encoded by the crRNA expression cassette.

34. The method of any one of claims 21-33, wherein the population of cells comprises effector T cells, memory T cells, central memory T cells, effector memory T cells, Th1 cells, Th2 cells, Th3 cells, Th9 cells, Th17 cells, Tfh cells, Treg cells, gamma-delta T cells, hematopoietic stem cells (HSC), macrophages, natural killer cells (NK), B cells, dendritic cells (DC), or other immune cells.

35. The method of claim 34, wherein the T cells are CD4⁺ or CD8⁺ T cells.

36. An isolated CAR T cell comprising a CAR and one or more mutations in one or more genes identified by the method of any one of claims 21-35.

37. The CAR T cell of claim 36, wherein the one or more mutations cause reduced function of the one or more genes or gene products thereof.

38. The CAR T cell of claim 36 or 37, wherein the one or more genes is selected from the group comprising PRDM1, DPF3, SLAMF1, TET2, HFE, PELI1, PDCD1, HAVCR2/TIM3, TET2, NR4A2, LAIR1, and USB1.

39. The CAR T cell of any one of claims 36-38, wherein the cell exhibits increased memory, increased cell proliferation, increased persistence, increased cytotoxicity towards a target cell, decreased T cell terminal differentiation, and/or reduced T cell exhaustion compared to a CAR T cell not comprising the one or more mutations in the one or more genes.

40. A population of CAR T cells derived by expanding the CAR T cell of any one of claims 36-39.

41. A pharmaceutical composition comprising the population of CAR T cells of claim 40 and a pharmaceutically acceptable buffer, carrier, diluent or excipient.

42. A method of treating a subject having a disease, disorder, or condition comprising administering to the subject an effective amount of the pharmaceutical composition of claim 41.

43. The method of claim 42, wherein the disease, disorder, or condition is associated with an elevated expression or specific expression of an antigen.

44. The method of claim 43, wherein the CAR T cell targets the antigen.

45. The method of any one of claims 42-44, wherein the cell was isolated from a healthy donor or from the subject having the disease, disorder, or condition prior to the introduction of the one or more mutations in the one or more genes.

46. The method of any one of claims 42-45, wherein the disease, disorder, or condition is a cancer, an inflammatory disease, a neuronal disorder, HIV/AIDS, diabetes, a cardiovascular disease, an infectious disease, or an autoimmune disease.

47. The method of claim 46, wherein the cancer is a leukemia or lymphoma selected from the group comprising chronic lymphocytic leukemia (CLL), acute lymphocytic leukemia (ALL), acute myeloid leukemia (AML), chronic myelogenous leukemia (CML), mantle cell lymphoma, non-Hodgkin's lymphoma, and Hodgkin's lymphoma.

48. The method of any one of claims 42-47, wherein the subject is a human.

49. A cell comprising a heterologous nucleic acid construct comprising one or more crRNA expression cassettes comprising a nucleic acid sequence encoding one or more guide RNAs selected from the group consisting of SEQ ID NOs:3-12,134 and a chimeric antigen receptor (CAR) expression cassette.

50. A cell comprising a heterologous nucleic acid construct encoding a chimeric antigen receptor (CAR) expression cassette and reduced or eliminated expression at one or more gene loci targeted by one or more guide RNAs selected from the group consisting of SEQ ID NOs:3-12,134 and.

51. The cell of claims 49 or 50, wherein the heterologous nucleic acid construct is present in the cell's genome at the TRAC gene locus, and optionally wherein the CAR is an anti-CD19 or anti-CD22 CAR.
